SEQUENCE LISTING (35 sequences)

SEQ ID NO: 1 - DNA, 4086 bp, Homo sapiens
gtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatagtgtaataattgtgcaggtacaggaattgttccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttgggtcaagactaactttcttttaaaggttcatttgtatgataaattcatatgtgtatatataattttttttgttttgtctagtgagtttcaacatttttaaagttttcaaaaagccatcggaatgttaaattaatgtaaagggacagctaatctagaccaaagaatggtattttcacttttctttgtaacattgaatggtttgaagtactcaaaatctgttacgctaaacttttgattctttaacacaattatttttaaacactggcattttccaaaactgtggcagctaactttttaaaatctcaaatgacatgcagtgtgagtagaaggaagtcaacaatatgtggggagagcactcggttgtctttacttttaaaagtaatacttggtgctaagaatttcaggattattgtatttacgttcaaatgaagatggcttttgtacttcctgtggacatgtagtaatgtctatattggctcataaaactaacctgaaaaacaaataaatgctttggaaatgtttcagttgctttagaaacattagtgcctgcctggatccccttagttttgaaatatttgccattgttgtttaaatacctatcactgtggtagagcttgcattgatcttttccacaagtattaaactgccaaaatgtgaatatgcaaagcctttctgaatctataataatggtacttctactggggagagtgtaatattttggactgctgttttccattaatgaggagagcaacaggcccctgattatacagttccaaagtaataagatgttaattgtaattcagccagaaagtacatgtctcccattgggaggatttggtgttaaataccaaactgctagccctagtattatggagatgaacatgatgatgtaacttgtaatagcagaatagttaatgaatgaaactagttcttataatttatctttatttaaaagcttagcctgccttaaaactagagatcaactttctcagctgcaaaagcttctagtctttcaagaagttcatactttatgaaattgcacagtaagcatttatttttcagaccatttttgaacatcactcctaaattaataaagtattcctctgttgctttagtatttattacaataaaaagggtttgaaatatagctgttctttatgcataaaacacccagctaggaccattactgccagagaaaaaaatcgtattgaatggccatttccctacttataagatgtctcaatctgaatttatttggctacactaaagaatgcagtatatttagttttccatttgcatgatgtttgtgtgctatagatgatattttaaattgaaaagtttgttttaaattatttttacagtgaagactgttttcagctctttttatattgtacatagtcttttatgtaatttactggcatatgttttgtagactgtttaatgactggatatcttccttcaacttttgaaatacaaaaccagtgttttttacttgtacactgttttaaagtctattaaaattgtcatttgacttttttctg

SEQ ID NO: 2 - DNA, 3849 bp, Mus musculus
gcggagcagaggaggcgagggcggagggccagagaggcagttggaagatggcggacgaggtggcgctcgcccttcaggccgccggctccccttccgcggcggccgccatggaggccgcgtcgcagccggcggacgagccgctccgcaagaggccccgccgagacgggcctggcctcgggcgcagcccgggcgagccgagcgcagcagtggcgccggcggccgcggggtgtgaggcggcgagcgccgcggccccggcggcgctgtggcgggaggcggcaggggcggcggcgagcgcggagcgggaggccccggcgacggccgtggccggggacggagacaatgggtccggcctgcggcgggagccgagggcggctgacgacttcgacgacgacgagggcgaggaggaggacgaggcggcggcggcagcggcggcggcagcgatcggctaccgagacaacctcctgttgaccgatggactcctcactaatggctttcattcctgtgaaagtgatgacgatgacagaacgtcacacgccagctctagtgactggactccgcggccgcggataggtccatatacttttgttcagcaacatctcatgattggcaccgatcctcgaacaattcttaaagatttattaccagaaacaattcctccacctgagctggatgatatgacgctgtggcagattgttattaatatcctttcagaaccaccaaagcggaaaaaaagaaaagatatcaatacaattgaagatgctgtgaagttactgcaggagtgtaaaaagataatagttctgactggagctggggtttctgtctcctgtgggattcctgacttcagatcaagagacggtatctatgctcgccttgcggtggacttcccagacctcccagaccctcaagccatgtttgatattgagtattttagaaaagacccaagaccattcttcaagtttgcaaaggaaatatatcccggacagttccagccgtctctgtgtcacaaattcatagctttgtcagataaggaaggaaaactacttcgaaattatactcaaaatatagataccttggagcaggttgcaggaatccaaaggatccttcagtgtcatggttcctttgcaacagcatcttgcctgatttgtaaatacaaagttgattgtgaagctgttcgtggagacatttttaatcaggtagttcctcggtgccctaggtgcccagctgatgagccacttgccatcatgaagccagagattgtcttctttggtgaaaacttaccagaacagtttcatagagccatgaagtatgacaaagatgaagttgacctcctcattgttattggatcttctctgaaagtgagaccagtagcactaattccaagttctataccccatgaagtgcctcaaatattaataaatagggaacctttgcctcatctacattttgatgtagagctccttggagactgcgatgttataattaatgagttgtgtcataggctaggtggtgaatatgccaaactttgttgtaaccctgtaaagctttcagaaattactgaaaaacctccacgcccacaaaaggaattggttcatttatcagagttgccaccaacacctcttcatatttcggaagactcaagttcacctgaaagaactgtaccacaagactcttctgtgattgctacacttgtagaccaagcaacaaacaacaatgttaatgatttagaagtatctgaatcaagttgtgtggaagaaaaaccacaagaagtacagactagtaggaatgttgagaacattaatgtggaaaatccagattttaaggctgttggttccagtactgcagacaaaaatgaaagaacttcagttgcagaaacagtgagaaaatgctggcctaatagacttgcaaaggagcagattagtaagcggcttgagggtaatcaatacctgtttgtaccaccaaatcgttacatattccacggtgctgaggtatactcagactctgaagatgacgtcttgtcctctagttcctgtggcagtaacagtgacagtggcacatgccagagtccaagtttagaagaacccttggaagatgaaagtgaaattgaagaattctacaatggcttggaagatgatacggagaggcccgaatgtgctggaggatctggatttggagctgatggaggggatcaagaggttgttaatgaagctatagctacaagacaggaattgacagatgtaaactatccatcagacaaatcataacactattgaagctgtccggattcaggaattgctccaccagcattgggaactttagcatgtcaaaaaaatgaatgtttacttgtgaacttgaacaaggaaatctgaaagatgtattatttatagactggaaaatagattgtcttcttggataatttctaaagttccatcatttctgtttgtacttgtacattcaacactgttggttgacttcatcttcctttcaaggttcatttgtatgatacattcgtatgtatgtataattttgttttttgcctaatgagtttcaaccttttaaagttttcaaaagccattggaatgttaatgtaaagggaacagcttatctagaccaaagaatggtatttcacacttttttgtttgtaacattgaatagtttaaagccctcaatttctgttctgctgaacttttatttttaggacagttaactttttaaacactggcattttccaaaacttgtggcagctaactttttaaaatcacagatgacttgtaatgtgaggagtcagcaccgtgtctggagcactcaaaacttgggctcagtgtgtgaagcgtacttactgcatcgtttttgtacttgctgcagacgtggtaatgtccaaacaggcccctgagactaatctgataaatgatttggaaatgtgtttcagttgttctagaaacaatagtgcctgtctatataggtccccttagtttgaatatttgccattgtttaattaaatacctatcactgtggtagagcctgcatagatcttcaccacaaatactgccaagatgtgaatatgcaaagcctttctgaatctaataatggtacttctactggggagagtgtaatattttggactgctgtttttccattaatgaggaaagcaataggcctcttaattaaagtcccaaagtcataagataaattgtagctcaaccagaaagtacactgttgcctgttgaggatttggtgtaatgtatcccaaggtgttagccttgtattatggagatgaatacagatccaatagtcaaatgaaactagttcttagttatttaaaagcttagcttgccttaaaactagggatcaattttctcaactgcagaaacttttagcctttcaaacagttcacacctcagaaagtcagtatttattttacagacttctttggaacattgcccccaaatttaaatattcatgtgggtttagtatttattacaaaaaaatgatttgaaatatagctgttctttatgcataaaatacccagttaggaccattactgccagaggagaaaagtattaagtagctcatttccctacctaaaagataactgaatttatttggctacactaaagaatgcagtatatttagttttccatttgcatgatgtgtttgtgctatagacaatattttaaattgaaaaatttgttttaaattatttttacagtgaagactgttttcagctctttttatattgtacatagacttttatgtaatctggcatatgttttgtagaccgtttaatgactggattatcttcctccaacttttgaaatacaaaaacagtgttttatactaaaaaaaaaaaaa

SEQ ID NO: 3 - DNA, 2103 bp, Rattus norvegicus
atgtgggccaggcaagtgtttactggaccctcagactgtggccccgacgagggaggggtcacaaagcctcagagagtaaatggacgactgaatctggcaccgcagacagctgcccggggccctgatagctgcgagggctccgcggcggcggcgcagctcggccctggctcggccgcgcgccgccgagctcgccagccctactgcggagccgcgcgcggactcgccgctcgcttgggccgccttgcgctgctgcgcgcagtcgccggggcagcaggctctcgcagttgtcacaacctcctgttggctgatgagatcatcactaatggctttcattcctgtgaaagtgatgacgatgacagagcatcacacgcaagctctagtgactggactccaaggccacggataggtccatatacttttgttcagcaacacctcatgattggcaccgatcctcgaacaattcttaaagatttattaccagaaacaattcctccacctgagttggatgatatgacactgtggcagattgttattaatatcctttcagaaccaccaaagcggaaaaaaagaaaagatattaatacaattgaagatgctgtgaagttactacaagagtgcaaaaagataatagttctgactggagctggggtttctgtttcctgtgggatacctgacttcagatcaagagatggtatttatgctcgccttgctgtggacttcccggatctcccagatcctcaagccatgttcgatattgagtattttagaaaagacccaagaccattcttcaagtttgcaaaggaaatatatcccggacagttccagccatctctgtgtcacaaattcatagctttgtcagataaggaaggaaaactacttcgaaattatactcaaaatatagataccttggagcaggttgcaggaatccaaaggatcattcagtgtcatggttcctttgcaacagcatcttgcctgatttgtaaatacaaagttgattgtgaagctgttcgtggagatatttttaatcaggtagttcctcggtgtcctaggtgcccagctgatgagccacttgccatcatgaagccagagattgtcttctttggtgaaaacttaccagaacagtttcatagagccatgaagtatgacaaagatgaagttgacctcctcattgttattgggtcttctctgaaagtaagaccagtagcactaattccaagttctataccccatgaagtgcctcaaatattaataaatagggaacctctgcctcatctacattttgatgtagagcttcttggagactgcgatgtcataattaatgagttgtgtcataggttaggtggcgagtatgccaaactttgttgtaaccctgtaaagctttcagaaattactgaaaaacctccacgaacacaaaaggaattggttcatttatcagagttgccaccaacacctcttcatatttcagaagactcaagttcacctgaaagaactgtaccacaagactcttctgtgattgctacacttgtagaccaaacaataaagaacaaagttgacgatttagaagtatctgaaccaaaaagttgtgtggaagaaaaatcacaggaagtacagacttataggaatgttgagagcattaatgtggaaaacccagatttcaaggctgttggttccagtactggagacaaaaatgaaagaacttctgttgcagaaacagtgagaaaatgctggcctaatagacttgcaaaggagcagattagtaagcgtcttgacggtaatcaatacttgtttgtaccaccaaatcgttacatatttcatggtgctgaggtatactcagactctgaagatgacgccttatcctctagttcctgtggcagtaacagtgacagtggcacatgccagagtccaagtttagaagaacccttggaagatgaaagtgaaattgaagagttctacaatggcttggaagatgatgctgacagaccggagtgtgctggaggatctggagctgacggaggggatcaagaggcagttaatgaagctatagccatgaaacaggaattaacagatgtaaactgtacaccagacaaatcagagcactattga

SEQ ID NO: 4 - DNA, 1963 bp, Homo sapiens
gtgttgtacgaaagcgcgtctgcggccgcaatgtctgctgagagttgtagttctgtgccctatcacggccactcccatttctggtgccgtcacgggacagagcagtcggtgacaggacagagcagtcggtgacgggacacagtggttggtgacgggacagagcggtcggtgacagcctcaagggcttcagcaccgcgcccatggcagagccagacccctctcaccctctggagacccaggcagggaaggtgcaggaggctcaggactcagattcagactctgagggaggagccgctggtggagaagcagacatggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccagtgacagctgcatctcccaggcgggatgccgagctcctcagggacagctgagccccaaccgggcctggccccctcttaaccagcagttcttgtctggggagctcagaacatcccccaatctcttacagctccctccccaaaactggggtcccagcaaccctggcccccaaccccagcaaatctctaacacctcctagaggccaaggcttaaacaggcatctctaccagccccactgtctctaaccactcctgggctaaggagtaacctccctcatctctaactgcccccacggggccagggctaccccagaacttttaactcttccaggacagggagcttcgggcccccactctgtctcctgcccccgggggcctgtggctaagtaaaccatacctaacctaccccagtgtgggtgtgggcctctgaatataacccacacccagcgtagggggagtctgagccgggagggctcccgagtctctgccttcagctcccaaagtgggtggtgggcccccttcacgtgggacccacttcccatgctggatgggcagaagacattgcttattggagacaaattaaaaacaaaaacaactaac

SEQ ID NO: 5 - DNA, 1826 bp, Mus musculus
atcctatctcggcctcttcttgtttccgctgccgtcacgggacagagcagtcggtgacagtcccgagggcccccaccccgttcccatggccgagccggacccctctgaccctctggagacccaggcagggaaggtgcaggaggctcaggattcagactcggacactgagggaggagccactggtggagaggcagagatggacttcctgaggaatttattcacccagaccctgggcctgggttcccaaaaggagcgtcttctagacgagctgaccctcgaaggagtgacacgctacatgcagagcgagcgctgccgcaaggtcatctgtttggtgggagccggaatctccacgtccgcgggtatccctgacttccgctccccgtccactggcctctatgcaaacctggagaagtaccaccttccttacccagaggccatctttgagatcagctacttcaagaaacatccggaacccttctttgcccttgccaaggagctctatcccgggcagttcaagccaaccatctgccactacttcatccgcctgctgaaggagaaggggctgctgctgcgctgctacacgcagaacatagacacgctggaacgagtggcggggctggagccccaggacctggtggaggcccacggcaccttctacacatcacactgtgtcaacacctcctgcagaaaagaatacacgatgggctggatgaaagagaagatcttctcagaagcaactcccaggtgtgagcagtgtcagagtgtggtaaagcctgatatcgtgtttttcggtgagaaccttccatcgcgcttcttctcctgcatgcagtcagacttctccaaggtggacctcctcatcatcatgggcacctccctgcaggtgcagcccttcgcctccctcatcagcaaggcaccactagccaccccacggctgctcattaacaaggaaaagacaggccagacggaccccttcctgggcatgatgatgggcctgggaggtggcatggattttgactccaagaaggcttacagggacgtggcctggctgggtgactgtgatcaaggctgcctggctctcgctgacctcctcggatggaagaaggaactggaagaccttgtccggagggagcatgccaacatagatgcccagtcagggtcacaggcccccaaccccagcactaccatctcccctggaaagtccccaccgcctgccaaggaggcggccaggaccaaagagaaagaggaacagcagtaacagtaaccatgacctcccgcaggacagcggacgccggccagcactgggccctcttaacatgcagcttgtgtgagctcaaagacccttcgttctttaaccacgttcttgaaatcagggtccccaactcaatcccagagaagcggaatatacctaagggctgaggcctgtgcagtctgtagctggggcctctaaccaccatagccgctaaccacccaggcaagaagcagccttccctaacttctaattattcccagacaacaggctaccccaaaacccctaacagtgccagaataaggcatttctctattgttttgagggggcctatggtaaatcaaattaacctaccccgcataggggctggactctacaaatagaacttcacccaagggggtggggccttgtgggatctctgagcctgaaggcctgccaactctctgcctccaacaaagtgggtactaggctccctttcctggggacccacttgccagctgttggtggatgagcaagagaccttgcttattagaaacaaattaaaaaacaaaacaaagcaactaaaaaaaaaaaaaaaaaa

SEQ ID NO: 6 - DNA, 1869 bp, Homo sapiens
ggcgccgggggcgggggtgggaggcggaggcggggccggggcgccgcgggcggggcgccgggggcggggcgagtccggaggactcctcggactgcgcggaacatggcgttctggggttggcgcgccgcggcagccctccggctgtggggccgggtagttgaacgggtcgaggccgggggaggcgtggggccgtttcaggcctgcggctgtcggctggtgcttggcggcagggacgatgtgagtgcggggctgagaggcagccatggggcccgcggtgagcccttggacccggcgcgccccttgcagaggcctcccagacccgaggtgcccagggcattccggaggcagccgagggcagcagctcccagtttcttcttttcgagtattaaaggtggaagaaggtccatatctttttctgtgggtgcttcaagtgttgttggaagtggaggcagcagtgacaaggggaagctttccctgcaggatgtagctgagctgattcgggccagagcctgccagagggtggtggtcatggtgggggccggcatcagcacacccagtggcattccagacttcagatcgccggggagtggcctgtacagcaacctccagcagtacgatctcccgtaccccgaggccatttttgaactcccattcttctttcacaaccccaagccctttttcactttggccaaggagctgtaccctggaaactacaagcccaacgtcactcactactttctccggctgcttcatgacaaggggctgcttctgcggctctacacgcagaacatcgatgggcttgagagagtgtcgggcatccctgcctcaaagctggttgaagctcatggaacctttgcctctgccacctgcacagtctgccaaagacccttcccaggggaggacattcgggctgacgtgatggcagacagggttccccgctgcccggtctgcaccggcgttgtgaagcccgacattgtgttctttggggagccgctgccccagaggttcttgctgcatgtggttgatttccccatggcagatctgctgctcatccttgggacctccctggaggtggagccttttgccagcttgaccgaggccgtgcggagctcagttccccgactgctcatcaaccgggacttggtggggcccttggcttggcatcctcgcagcagggacgtggcccagctgggggacgtggttcacggcgtggaaagcctagtggagcttctgggctggacagaagagatgcgggaccttgtgcagcgggaaactgggaagcttgatggaccagacaaataggatgatggctgcccccacacaataaatggtaacataggagacatccacatcccaattctgacaagacctcatgcctgaagacagcttgggcaggtgaaaccagaatatgtgaactgagtggacacccgaggctgccactggaatgtcttctcaggccatgagctgcagtgactggtagggctgtgtttacagtcagggccaccccgtcacatatacaaaggagctgcctgcctgtttgctgtgttgaactcttcactctgctgaagctcctaatggaaaaagctttcttctgactgtgaccctcttgaactgaatcagaccaactggaatcccagaccgagtctgctttctgtgcctagttgaacggcaagctcggcatctgttggttacaagatccagacttgggccgagcggtccccagccctcttcatgttccgaagtgtagtcttgaggccctggtgccgcacttctagcatgttggtctcctttagtggggctatttttaatgagagaaaatctgttctttccagcatgaaatacatttagtctcctcaaaaaaaaaaaaca

SEQ ID NO: 7 - DNA, 1428 bp, Mus musculus
ggggattcggatggcgcttgaccctctaggcgccgtcgtcctgcagagcatcatggcgctaagcggtcgactggcattggccgcgctcagactgtggggtccgggagtgttacaggtgggagaaggcccatatccctctgtgtgggagcctcaggcggctttggaggtggaggaagcagtgagaagaagttttctctgcaggatgtagctgagctgcttcggaccagagcctgcagtagggtggtggtcatggtgggggccggcatcagcacacccagtggcatcccggacttcagatccccagggagcggcctctacagcaaccttcagcagtatgacatcccgtaccctgaagccatctttgaacttggctttttctttcacaaccccaagccctttttcatgttggccaaggagctgtaccctgggcactacaggcccaatgtcactcactacttcctgaggctcctccacgacaaggagctgcttctgcggctctatacacagaacatcgacgggcttgagagagcatctgggatccctgcctcaaagctggttgaagcccacgggacctttgtaacagctacatgcacggtctgtcgaaggtccttcccaggggaagacatatgggctgatgtgatggcggacagggtgccccgctgcgctgtctgtactggcgttgtgaaacccgacattgtgttctttggggagcagctgcctgcaaggttcctactccatatggctgacttcgctttggcagatctgctactcattcttgggacctccctggaggtggagccttttgccagcttgtctgaagcagtacagaaatcagtgccccgactgctcatcaatcgagacttggtggggccgttcgttctgagtcctcgaaggaaagatgtggtccagctaggggatgtagttcatggtgtggaaaggctggtggacctcctggggtggacacaagaactgctggatcttatgcagcgggaacgtggcaagctggatggacaggacagataagactatggcttcttcacctggggaagtcacacagcagatcatcctatgtccagcaagacttcatgcctgaagacagctccaacacgtttacaaacatgaaccagaccacaacatgtggcctggacagtggtcctccgaggctgcctttggaaaggctgaccagggatgtctacccttggggcccctccatgtgtgcgccctgtccacctcatcactgctgaaggtgtagtgcaggtgctgctttctgcagcggcccttaagttatcacgagggcagcacagcacgcccgtcgccaggcaggcgatgcactagggcaatctagcatgttgatcggtaaagtggcatctttaactacaacatcatttcttgcatgaaataaacttagtataaaaaaaaaaaaaaaaa

SEQ ID NO: 8 - DNA, 1473 bp, Mus musculus
acttccgctaaacttctcccgggtttctggccctgcccttgaggcattaaagagtagaggtgcctggggacagctagccactgactggtcacgtagcctcaagcctgcagacttgggtcctctgaaaccggatggcgtttggcgaggactagtgttacaggtgggagaaggcccatatccctctgtgtgggagcctcaggcggctttggaggtggaggaagcagtgagaagaagttttctctgcaggatgtagctgagctgcttcggaccagagcctgcagtagggtggtggtcatggtgggggccggcatcagcacacccagtggcatcccggacttcagatccccagggagcggcctctacagcaaccttcagcagtatgacatcccgtaccctgaagccatctttgaacttggctttttctttcacaaccccaagccctttttcatgttggccaaggagctgtaccctgggcactacaggcccaatgtcactcactacttcctgaggctcctccacgacaaggagctgcttctgcggctctatacacagaacatcgacgggcttgagagagcatctgggatccctgcctcaaagctggttgaagcccacgggacctttgtaacagctacatgcacggtctgtcgaaggtccttcccaggggaagacatatgggctgatgtgatggcggacagggtgccccgctgcgctgtctgtactggcgttgtgaaacccgacattgtgttctttggggagcagctgcctgcaaggttcctactccatatggctgacttcgctttggcagatctgctactcattcttgggacctccctggaggtggagccttttgccagcttgtctgaagcagtacagaaatcagtgccccgactgctcatcaatcgagacttggtggggccgttcgttctgagtcctcgaaggaaagatgtggtccagctaggggatgtagttcatggtgtggaaaggctggtggacctcctggggtggacacaagaactgctggatcttatgcagcgggaacgtggcaagctggatggacaggacagataagactatggcttcttcacctggggaagtcacacagcagatcatcctatgtccagcaagacttcatgcctgaagacagctccaacacgtttacaaacatgaaccagaccacaacatgtggcctggacagtggtcctccgaggctgcctttggaaaggctgaccagggatgtctacccttggggcccctccatgtgtgcgccctgtccacctcatcactgctgaaggtgtagtgcaggtgctgctttctgcagcggcccttaagttatcacgagggcagcacagcacgcccgtcgccaggcaggcgatgcactagggcaatctagcatgttgatcggtaaagtggcatctttaactacaacatcatttcttgcatgaaataaacttagtataaaaaaaaaaaaaaaaa

SEQ ID NO: 9 - DNA, 1174 bp, Homo sapiens
gtccgtagagctgtgagagaatgaagatgagctttgcgttgactttcaggtcagcaaaaggccgttggatcgcaaaccccagccagccgtgctcgaaagcctccattgggttatttgtgccagcaagtcctcctctggaccctgagaaggtcaaagagttacagcgcttcatcaccctttccaagagactccttgtgatgactggggcaggaatctccaccgaatcggggataccagactacaggtcagaaaaagtggggctttatgcccgcactgaccgcaggcccatccagcatggtgattttgtccggagtgccccaatccgccagcggtactgggcgagaaacttcgtaggctggcctcaattctcctcccaccagcctaaccctgcacactgggctttgagcacctgggagaaactcggaaagctgtactggttggtgacccaaaatgtggatgctttgcacaccaaggcggggagtcggcgcctgacagagctccacggatgcatggacagggtcctgtgcttggattgtggggaacagactccccggggggtgctgcaagagcgtttccaagtcctgaaccccacctggagtgctgaggcccatggcctggctcctgatggtgacgtctttctctcagaggagcaagtccggagctttcaggtcccaacctgcgttcaatgtggaggccatctgaaaccagatgtcgttttcttcggggacacagtgaaccctgacaaggttgattttgtgcacaagcgtgtaaaagaagccgactccctcttggtggtgggatcatccttgcaggtatactctggttacaggtttatcctcactgcctgggagaagaagctcccgattgcaatactgaacattgggcccacacggtcggatgacttggcgtgtctgaaactgaattctcgttgtggagagttgctgcctttgatagacccatgctgaccacagcctgatattccagaacctggaacagggactttcacttgaatcttgctgctaaatgtaaatgccttctcaaatgacagattccagttcccattcaacagagtagggtgcactgacaaagtatagaaggttctaggtatcttaatgtgtggatattcttaattaaaactcattttttttaaataaaaaattgttcagctttaaaa

SEQ ID NO: 10 - DNA, 1633 bp, Homo sapiens
cgcctctaggagaaagcctggaacgcgtaccggagggtaccagagctcttagcgggccggcagcatgtgcggggccaagtaaatggaaatgttttctaacatataaaaacctacagaagaagaaaataattttctggatcaaattagaagtctgtattatattgatgtctccagattcaaatatattagaaagcagccgtggagacaaccatcttcattttgggagaaataactaaagcccgcctcaagcattagaactacagacaaaccctgatgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaaaatggcaagcccaggacctggcgactcccctggcctttgcccacaacccgtcccgggtgtgggagttctaccactaccggcgggaggtcatggggagcaaggagcccaacgccgggcaccgcgccatagccgagtgtgagacccggctgggcaagcagggccggcgagtcgtggtcatcacccagaacatcgatgagctgcaccgcaaggctggcaccaagaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcaggtttcatttccagggaccctgtggaacgactcttcctgaagcccttgcctgtcatgaaaatgaaactgtttcttaagtgtcctggggaagaaagaaattacagtatatctaagaactaggccacacgcagaggagaaatggtcttatgggtggtgagctgagtactgaacaatctaaaaatagcctctgattccctcgctggaatccaacctgttgataagtgatgggggtttagaagtagcaaagagcacccacattcaaaagtcacagaactggaaagttaattcatattatttggtttgaactgaaacgtgaggtatctttgatgtgtatggttggttattgggagggaaaaattttgtaaattagattgtctaaaaaaaatagttattctgattatatttttgttatctgggcaaagtagaagtcaaggggtaaaaaccctactattctgatttttgcacaagttttagtggaaaataaaatcacactctacagtaggt

SEQ ID NO: 11 - DNA, 1638 bp, Homo sapiens
gcttccggcggaagcggcctcaacaagggaaactttattgttcccgtggggcagtcgaggatgtcggtgaattacgcggcggggctgtcgccgtacgcggacaagggcaagtgcggcctcccggagatcttcgaccccccggaggagctggagcggaaggtgtgggaactggcgaggctggtctggcagtcttccagtgtggtgttccacacgggtgccggcatcagcactgcctctggcatccccgacttcaggggtccccacggagtctggaccatggaggagcgaggtctggcccccaagttcgacaccacctttgagagcgcgcggcccacgcagacccacatggcgctggtgcagctggagcgcgtgggcctcctccgcttcctggtcagccagaacgtggacgggctccatgtgcgctcaggcttccccagggacaaactggcagagctccacgggaacatgtttgtggaagaatgtgccaagtgtaagacgcagtacgtccgagacacagtcgtgggcaccatgggcctgaaggccacgggccggctctgcaccgtggctaaggcaagggggctgcgagcctgcaggggagagctgagggacaccatcctagactgggaggactccctgcccgaccgggacctggcactcgccgatgaggccagcaggaacgccgacctgtccatcacgctgggtacatcgctgcagatccggcccagcgggaacctgccgctggctaccaagcgccggggaggccgcctggtcatcgtcaacctgcagcccaccaagcacgaccgccatgctgacctccgcatccatggctacgttgacgaggtcatgacccggctcatggagcacctggggctggagatccccgcctgggacggcccccgtgtgctggagagggcgctgccacccctgccccgcccgcccacccccaagctggagcccaaggaggaatctcccacccggatcaacggctctatccccgccggccccaagcaggagccctgcgcccagcacaacggctcagagcccgccagccccaaacgggagcggcccaccagccctgccccccacagaccccccaaaagggtgaaggccaaggcggtccccagctgaccagggtgcttggggagggtggggctttttgtagaaactgtggattctttttctctcgtggtctcactttgttacttgtttctgtccccgggagcctcagggctctgagagctgtgctccaggccaggggttacacctgccctccgtggtccctccctgggctccaggggcctctggtgcggttccgggaagaagccacaccccagaggtgacagctgagcccctgccacaccccagcctctgacttgctgtgttgtccagaggtgaggctgggccctccctggtctccagcttaaacaggagtgaactccctctgtccccagggcctcccttctgggccccctacagcccaccctacccctcctccatgggccctgcaggaggggagacccaccttgaagtgggggatcagtagaggcttgcactgcctttggggctggagggagacgtgggtccaccaggcttctggaaaagtcctcaatgcaataaaaacaatttctttcttgcaaa

SEQ ID NO: 12 - DNA, 1682 bp, Mus musculus
cgtgcggcagcgccggcgacgatgtcggtgaattatgcagcagggttgtcgccttacgcggataagggcaagtgcgggctgcccgagatcttcgacccaccagaggagctggaacgcaaggtgtgggagctggcccggctaatgtggcagtcctccagcgtggttttccacacgggcgccggcatcagcaccgcctctggcatccccgacttcagaggcccccatggcgtgtggaccatggaggaacgcggcctggcccccaagtttgacaccaccttcgagaatgctcggccctcgaagacccacatggccctggttcagctagaacgcatgggcttcctcagcttcctggtcagccagaacgtagacgggctgcacgtgcgctcgggcttccccagggacaagctggcagagctgcacggaaacatgtttgtagaggaatgtcccaagtgtaagacgcagtacgtcagagacacggttgtgggcaccatgggcctcaaggccacaggccggctctgcaccgtggccaagaccaggggacttcgggcctgtagaggggagctgagagacaccattctggactgggaggactcgttgcctgaccgggacctgatgctcgctgatgaggccagcaggaccgcagacctgtctgtcaccctgggtacctcgctgcagatccgccccagtgggaacctgccccttgccactaagcgccgaggaggccgtctggtcattgtcaacctgcaacccacaaaacatgaccgccaggctgacctgcgcatccacggctacgtggatgaggtgatgtgcagactcatgaagcatctggggctggagattccagcctgggatggaccctgcgtgctagacaaagccctgccacctctgcctcgcccagtagcactcaaggctgagccccccgtgcatctcaatggtgcagtgcatgtttcgtataagtccaagcccaacagccctatactccacaggccccccaaaagagtgaagaccgaggctgcccccagctgacccgggtatgtggggcgggaggggaacttgaaggaaccacacatgttgaggtttttgtttgtttgtttgtttgtttgttttttatattctggggactgagcccaggtttgcatatctcaccctggtgtcttttcagattttttttttttttttttttttttttgagaccaggactccctagatcgctcaggctggtctggaactcactgctatagcccaggctggcctggaactagcctgcctgcctcagcctctactgtagtagggacagcgggcaggtacaccatgtccaacacagctcctttcttcaaagtctcactgtgtcccttgtccaggtccttccagggaatctcagggcatggggaatcccacacatgtggaagaggccgtgtcccagggatccacgtgcctctctaccaatcacacccgctcgtgactcaggatgtgcccggagcagcagtctcacacactccacacaccattgctgccccctgcacgcatctcccccccatcccctggccctgacacagagccaccaagggcccaccaggcacccatctgtgctaagggccccaggctcccagaagctcccaatgcaataaatacaaaatcccttccattcttttaaaaaaaaaaaaaaaaaaaaaaaaaaaaaa

SEQ ID NO: 13 - DNA, 1718 bp, Homo sapiens
gcggaagcggaagagcaggtctccaggggagcgatggcagccgggggtctgagccgctccgagcgcaaagcggcggagcgggtccggaggttgcgggaggagcagcagagggagcgcctccgccaggtgtcgcgcatcctgaggaaggcggcggcggagcgcagcgccgaggagggccggctgctggccgagagcgcggacctggtaacggagctgcagggccggagccggcggcgcgagggcctgaagcggcggcaggaggaggtgtgcgacgacccggaggagctgcgggggaaggtccgggagctggccagcgccgtccggaacgccaaatacttggtcgtctacacaggcgcgggaatcagcacggcagcgtctatcccagactaccggggccctaatggagtgtggacactgcttcagaaagggagaagcgttagtgctgccgacctgagcgaggccgagccaaccctcacccacatgagcatcacccgtctgcatgagcagaagctggtgcagcatgtggtgtctcagaactgtgacgggctccacctgaggagtgggctgccgcgcacggccatctccgagctccacgggaacatgtacattgaagtctgtacctcctgcgttcccaacagggagtacgtgcgggtgttcgatgtgacggagcgcactgccctccacagacaccagacaggccggacctgccacaagtgtgggacccagctgcgggacaccattgtgcactttggggagagggggacgttggggcagcctctgaactgggaagcggcgaccgaggctgccagcagagcagacaccatcctgtgtctagggtccagcctgaaggttctaaagaagtacccacgcctctggtgcatgaccaagccccctagccggcggccgaagctttacatcgtgaacctgcagtggaccccgaaggatgactgggctgccctgaagctacatgggaagtgtgatgacgtcatgcggctcctcatggccgagctgggcttggagatccccgcctatagcaggtggcaggatcccattttctcactggcgactcccctgcgtgctggtgaagaaggcagccacagtcggaagtcgctgtgcagaagcagagaggaggccccgcctggggaccggggtgcaccgcttagctcggcccccatcctagggggctggtttggcaggggctgcacaaaacgcacaaaaaggaagaaagtgacgtaatcacgtgctcgatgaagaacagttggcactttgcagatggccagtgtcacggtgaaggctgggttgcccccacgggtctagggagaacgaactctttggggatgacattttcaccgtgacatttttagccatttgtccttgaggaagccccttgcactgctgcggttgtaccctgatacggcctggccatcgaggacacctgcccatccggcctctgtgtcaagaggtggcagccgcacctttctgtgagaacggaactcgggttatttcagccccggcctgcagagtggaagcgcccagcggcctttcctcgctcaccaggccagtctcagggcctcaccgtatttctactactacttaatgaaaaagtgtgaactttatagaatcctctctgtactggatgtgcggcagaggggtggctccgagcctcggctctatgcagacctttttatttctattaaacgtttctgcactggcaaaaa

SEQ ID NO: 14 - DNA, 1712 bp, Mus musculus
agccggtggcggtctgagccgctcggagcgcaaagctgctgagcgggtccggaggctgcgggaggagcagcagcgggagcgcctccgccaggtgtcacgcatcctgaggaaggcggctgcagagcgcagcgcggaggagggccggctcttggccgagagcgaggatctggtgaccgagctgcagggtcgaagtcggcggcgtgagggcctcaagcgccgccaggaggaggtgtgtgatgacccggaggagctgcggaggaaggtccgcgaactggccggagctgtccgaagtgccaggcacttggttgtctacacgggcgctggaatcagcacagcagcttctatcccagattatcggggtcctaatggagtatggacactgcttcagaaaggaaggcctgtgagtgctgccgacctaagcgaagcggagcctaccctcacccacatgagcatcacccgtttgcatgagcaaaagctggtgcaacacgtggtgtctcagaactgtgatgggctccacctgcggagtgggttgccccggaccgccatctcagagctccatgggaatatgtatattgaagtctgcacctcctgcatccctaacagagagtatgtacgagtgtttgatgtgactgagcgtactgcccttcaccgacacctgacaggccggacctgccacaagtgcgggacccagcttcgggataccattgtgcactttggggagagggggacattagggcagcctctgaactgggaggcagcgaccgaggctgctagcaaagcagacacaatcctgtgtttagggtccagcttgaaggtactaaagaaatatccccgcctctggtgcatgacgaagcctccaagccgtcgacccaaactctacatcgtgaacctgcagtggaccccgaaggatgactgggctgccctgaaactccatgggaagtgtgatgatgtcatgcaactcctcatgaatgaactgggcctggagattcctgtctacaaccggtggcaggatccaatcttctccttggcgacccccctccgtgctggtgaagaaggcagccacagtaggaagtcactatgcagaagcagagaagaagccccacctggggaccagagtgacccccttgcctcagctccccctatcctaggaggctggtttggcaggggttgtgccaagcgtgcaaaaaggaagaaagtggcatagcccggagttgacaaagaacagttggcactttgcagatggcttgacccttgttcaggggctccgcccgtgtcgccgtgaaggcgagagtgccccctccggctctagtgggtgccggcttcgcggtgacggtcgtctttagccatttgtccttgtggaaaggctctcttgcactgctgtgactgccccgctatgggcttgatcttcaaggacacctccctcctgttcacccctgtacaaaggtggcagcaaggcctctgtgagaagagctccggcccttcccaggcctggccagcaggagagtccaccactgccttacctcactctccaggctagtctcagggcctccctctttctactccttatcacgtacaagtgttcactttatagaagcctttttctgtattgggtatgcggcacagggagaatctaactaagcctccagtctgagcaagtcttattaaacatctctcaattgctaaaaaaaaaaaaaaaaaaaaaaaaaaaaaaa

SEQ ID NO: 15 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 16 - protein, 389 aa, Homo sapiens
MAEPDPSHPLETQAGKVQEAQDSDSDSEGGAAGGEADMDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 17 - protein, 352 aa, Homo sapiens
MDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 18 - protein, 399 aa, Homo sapiens
MAFWGWRAAAALRLWGRVVERVEAGGGVGPFQACGCRLVLGGRDDVSAGLRGSHGARGEPLDPARPLQRPPRPEVPRAFRRQPRAAAPSFFFSSIKGGRRSISFSVGASSVVGSGGSSDKGKLSLQDVAELIRARACQRVVVMVGAGISTPSGIPDFRSPGSGLYSNLQQYDLPYPEAIFELPFFFHNPKPFFTLAKELYPGNYKPNVTHYFLRLLHDKGLLLRLYTQNIDGLERVSGIPASKLVEAHGTFASATCTVCQRPFPGEDIRADVMADRVPRCPVCTGVVKPDIVFFGEPLPQRFLLHVVDFPMADLLLILGTSLEVEPFASLTEAVRSSVPRLLINRDLVGPLAWHPRSRDVAQLGDVVHGVESLVELLGWTEEMRDLVQRETGKLDGPDK

SEQ ID NO: 19 - protein, 314 aa, Homo sapiens
MKMSFALTFRSAKGRWIANPSQPCSKASIGLFVPASPPLDPEKVKELQRFITLSKRLLVMTGAGISTESGIPDYRSEKVGLYARTDRRPIQHGDFVRSAPIRQRYWARNFVGWPQFSSHQPNPAHWALSTWEKLGKLYWLVTQNVDALHTKAGSRRLTELHGCMDRVLCLDCGEQTPRGVLQERFQVLNPTWSAEAHGLAPDGDVFLSEEQVRSFQVPTCVQCGGHLKPDVVFFGDTVNPDKVDFVHKRVKEADSLLVVGSSLQVYSGYRFILTAWEKKLPIAILNIGPTRSDDLACLKLNSRCGELLPLIDPC

SEQ ID NO: 20 - protein, 310 aa, Homo sapiens
MRPLQIVPSRLISQLYCGLKPPASTRNQICLKMARPSSSMADFRKFFAKAKHIVIISGAGVSAESGVPTFRGAGGYWRKWQAQDLATPLAFAHNPSRVWEFYHYRREVMGSKEPNAGHRAIAECETRLGKQGRRVVVITQNIDELHRKAGTKNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFRFHFQGPCGTTLPEALACHENETVS

SEQ ID NO: 21 - protein, 299 aa, Homo sapiens
MRPLQIVPSRLISQLYCGLKPPASTRNQICLKMARPSSSMADFRKFFAKAKHIVIISGAGVSAESGVPTFRGAGGYWRKWQAQDLATPLAFAHNPSRVWEFYHYRREVMGSKEPNAGHRAIAECETRLGKQGRRVVVITQNIDELHRKAGTKNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFSHLISISSLIIIKN

SEQ ID NO: 22 - protein, 355 aa, Homo sapiens
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMEHLGLEIPAWDGPRVLERALPPLPRPPTPKLEPKEESPTRINGSIPAGPKQEPCAQHNGSEPASPKRERPTSPAPHRPPKRVKAKAVPS

SEQ ID NO: 23 - protein, 400 aa, Homo sapiens
MAAGGLSRSERKAAERVRRLREEQQRERLRQVSRILRKAAAERSAEEGRLLAESADLVTELQGRSRRREGLKRRQEEVCDDPEELRGKVRELASAVRNAKYLVVYTGAGISTAASIPDYRGPNGVWTLLQKGRSVSAADLSEAEPTLTHMSITRLHEQKLVQHVVSQNCDGLHLRSGLPRTAISELHGNMYIEVCTSCVPNREYVRVFDVTERTALHRHQTGRTCHKCGTQLRDTIVHFGERGTLGQPLNWEAATEAASRADTILCLGSSLKVLKKYPRLWCMTKPPSRRPKLYIVNLQWTPKDDWAALKLHGKCDDVMRLLMAELGLEIPAYSRWQDPIFSLATPLRAGEEGSHSRKSLCRSREEAPPGDRGAPLSSAPILGGWFGRGCTKRTKRKKVT

SEQ ID NO: 24 - DNA, 19 bp, Artificial Sequence
  Control vector
gatgaagttgacctcctca

SEQ ID NO: 25 - DNA, 19 bp, Artificial Sequence
  Control vector
gatgaagtcgacctcctca

SEQ ID NO: 26 - DNA, 28 bp, Artificial Sequence
  Primer
aaattcagaagaaagtaaacacattatt

SEQ ID NO: 27 - DNA, 17 bp, Artificial Sequence
  Primer
atgccctgaccatgtga

SEQ ID NO: 28 - DNA, 22 bp, Artificial Sequence
  Primer
gagcaaggtcttcatcattacg

SEQ ID NO: 29 - DNA, 19 bp, Artificial Sequence
  Primer
cccctggagctggagttac

SEQ ID NO: 30 - DNA, 20 bp, Artificial Sequence
  Promoter
ctctcccaccctcgccatac

SEQ ID NO: 31 - DNA, 21 bp, Artificial Sequence
  Promoter
ttgccagagaagccagtgaca

SEQ ID NO: 32 - protein, 798 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 33 - protein, 475 aa, Homo sapiens
MVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 34 - protein, 505 aa, Homo sapiens
MGETLGDSPIDPESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 35 - protein, 2440 aa, Homo sapiens
MSSSGYPPNQGAFSTEQSRYPPHSVQYTFPNTRHQQEFAVPDYRSSHLEVSQASQLLQQQQQQQLRRRPSLLSEFHPGSDRPQERRTSYEPFHPGPSPVDHDSLESKRPRLEQVSDSHFQRVSAAVLPLVHPLPEGLRASADAKKDPAFGGKHEAPSSPISGQPCGDDQNASPSKLSKEELIQSMDRVDREIAKVEQQILKLKKKQQQLEEEAAKPPEPEKPVSPPPVEQKHRSIVQIIYDENRKKAEEAHKIFEGLGPKVELPLYNQPSDTKVYHENIKTNQVMRKKLILFFKRRNHARKQREQKICQRYDQLMEAWEKKVDRIENNPRRKAKESKTREYYEKQFPEIRKQREQQERFQRVGQRGAGLSATIARSEHEISEIIDGLSEQENNEKQMRQLSVIPPMMFDAEQRRVKFINMNGLMEDPMKVYKDRQFMNVWTDHEKEIFKDKFIQHPKNFGLIASYLERKSVPDCVLYYYLTKKNENYKALVRRNYGKRRGRNQQIARPSQEEKVEEKEEDKAEKTEKKEEEKKDEEEKDEKEDSKENTKEKDKIDGTAEETEEREQATPRGRKTANSQGRRKGRITRSMTNEAAAASAAAAAATEEPPPPLPPPPEPISTEPVETSRWTEEEMEVAKKGLVEHGRNWAAIAKMVGTKSEAQCKNFYFNYKRRHNLDNLLQQHKQKTSRKPREERDVSQCESVASTVSAQEDEDIEASNEEENPEDSEVEAVKPSEDSPENATSRGNTEPAVELEPTTETAPSTSPSLAVPSTKPAEDESVETQVNDSISAETAEQMDVDQQEHSAEEGSVCDPPPATKADSVDVEVRVPENHASKVEGDNTKERDLDRASEKVEPRDEDLVVAQQINAQRPEPQSDNDSSATCSADEDVDGEPERQRMFPMDSKPSLLNPTGSILVSSPLKPNPLDLPQLQHRAAVIPPMVSCTPCNIPIGTPVSGYALYQRHIKAMHESALLEEQRQRQEQIDLECRSSTSPCGTSKSPNREWEVLQPAPHQVITNLPEGVRLPTTRPTRPPPPLIPSSKTTVASEKPSFIMGGSISQGTPGTYLTSHNQASYTQETPKPSVGSISLGLPRQQESAKSATLPYIKQEEFSPRSQNSQPEGLLVRAQHEGVVRGTAGAIQEGSITRGTPTSKISVESIPSLRGSITQGTPALPQTGIPTEALVKGSISRMPIEDSSPEKGREEAASKGHVIYEGKSGHILSYDNIKNAREGTRSPRTAHEISLKRSYESVEGNIKQGMSMRESPVSAPLEGLICRALPRGSPHSDLKERTVLSGSIMQGTPRATTESFEDGLKYPKQIKRESPPIRAFEGAITKGKPYDGITTIKEMGRSIHEIPRQDILTQESRKTPEVVQSTRPIIEGSISQGTPIKFDNNSGQSAIKHNVKSLITGPSKLSRGMPPLEIVPENIKVVERGKYEDVKAGETVRSRHTSVVSSGPSVLRSTLHEAPKAQLSPGIYDDTSARRTPVSYQNTMSRGSPMMNRTSDVTISSNKSTNHERKSTLTPTQRESIPAKSPVPGVDPVVSHSPFDPHHRGSTAGEVYRSHLPTHLDPAMPFHRALDPAAAAYLFQRQLSPTPGYPSQYQLYAMENTRQTILNDYITSQQMQVNLRPDVARGLSPREQPLGLPYPATRGIIDLTNMPPTILVPHPGGTSTPPMDRITYIPGTQITFPPRPYNSASMSPGHPTHLAAAASAEREREREREKERERERIAAASSDLYLRPGSEQPGRPGSHGYVRSPSPSVRTQETMLQQRPSVFQGTNGTSVITPLDPTAQLRIMPLPAGGPSISQGLPASRYNTAADALAALVDAAASAPQMDVSKTKESKHEAARLEENLRSRSAAVSEQQQLEQKTLEVEKRSVQCLYTSSAFPSGKPQPHSSVVYSEAGKDKGPPPKSRYEEELRTRGKTTITAANFIDVIITRQIASDKDARERGSQSSDSSSSLSSHRYETPSDAIEVISPASSPAPPQEKLQTYQPEVVKANQAENDPTRQYEGPLHHYRPQQESPSPQQQLPPSSQAEGMGQVPRTHRLITLADHICQIITQDFARNQVSSQTPQQPPTSTFQNSPSALVSTPVRTKTSNRYSPESQAQSVHHQRPGSRVSPENLVDKSRGSRPGKSPERSHVSSEPYEPISPPQVPVVHEKQDSLLLLSQRGAEPAEQRNDARSPGSISYLPSFFTKLENTSPMVKSKKQEIFRKLNSSGGGDSDMAAAQPGTEIFNLPAVTTSGSVSSRGHSFADPASNLGLEDIIRKALMGSFDDKVEDHGVVMSQPMGVVPGTANTSVVTSGETRREEGDPSPHSGGVCKPKLISKSNSRKSKSPIPGQGYLGTERPSSVSSVHSEGDYHRQTPGWAWEDRPSSTGSTQFPYNPLTMRMLSSTPPTPIACAPSAVNQAAPHQQNRIWEREPAPLLSAQYETLSDSDD

(19) United States
(12) Patent Application Publication
(10) Pub. No.: US 2005/0136429 A1
(43) Pub. Date: Jun. 23, 2005

(54) SIRT1 modulation of adipogenesis and adipose function

(75) Inventors: Leonard P. Guarente, Chestnut Hill, MA (US); Frederic Picard, Levis (CA)

Correspondence Address: FISH & RICHARDSON PC, 225 FRANKLIN ST, BOSTON, MA, 02110, US

(73) Assignee: MASSACHUSETTS INSTITUTE OF TECHNOLOGY

(21) Appl. No.: 10/885,977

(22) Filed: Jul. 6, 2004

Related U.S. Application Data
(60) Provisional application No. 60/484,836, filed on Jul. 3, 2003.

(51) Int. Cl. C12Q 1/68; A61K 48/00; C12N 5/08

(52) U.S. Cl. 435/6; 435/366; 424/93.21

(57) ABSTRACT

SIRT1 regulates the physiology of cells of the adipocyte lineage. Modulators of SIRT1 activity can be used to ameliorate, treat, or prevent diseases and disorders associated with adipose physiology, e.g., obesity, an obesity-related disease, or a fat-related metabolic disorder.

### CROSS REFERENCE TO RELATED APPLICATIONS

This application claims priority to 60/484,836, filed Jul. 3, 2003, the contents of which are hereby incorporated by reference in its entirety.

### GOVERNMENT SUPPORT

The invention was supported, in whole or in part, with United States Government support under Contract Numbers AG21150-01; AG11119-11; and AG15339-04 awarded by the National Institutes of Health. The United States has certain rights in the invention.

### BACKGROUND

Vertebrates possess two distinct types of adipose tissue: white adipose tissue (WAT) and brown adipose tissue (BAT). WAT stores and releases fat according to the nutritional needs of the animal. BAT burns fat and releases energy in the form of nonshivering heat. In vertebrates, a chronic positive energy balance translates into an increase in WAT fat depots and obesity.

Adipocytes develop from fibroblast-like cells, both during normal mammalian development and in various pathological circumstances, e.g., muscular dystrophy, where the muscle cells die and are gradually replaced by fatty connective tissue. See e,g., Sul (1989) Curr. Opin. Cell Biol. 1:1116-1121.

### SUMMARY

SIRT1 regulates the physiology of cells of the adipocyte lineage. Modulators of SIRT1 activity can be used to ameliorate, treat, or prevent diseases and disorders associated with adipose physiology, e.g., obesity, an obesity-related disease, or a fat-related metabolic disorder.

The SIR2 gene family has diverse functions in yeast including gene silencing, DNA repair, cell-cycle progression, and chromosome fidelity in meiosis and aging. Mammalian homologs of SIR2 proteins are called sirtuins and are a homologous family of proteins. Many of these proteins can function as NAD-dependent protein deacetylases. The protein product of the gene hSIR2 (SIRT1) is the human homolog of the S. cerevisiae Sir2 protein known to be involved in cell aging. Human SIRT1 mRNA is disclosed at GenBank Accession No. AF083106. It has been observed that expression of wild-type hSir2 in human cells reduces the transcriptional activity of p53. See, e.g., Vaziri et al., Cell Oct. 19, 2001;107 (2):149-59.

In one aspect, the disclosure features a method that includes: evaluating a SIRT1 molecule from a subject; and, optionally, recording information from the SIRT1 evaluation in association with metabolic information about the subject. The method can be used to evaluate a subject. In one embodiment, the SIRT1 molecule is SIRT1 mRNA, cDNA, or genomic nucleic acid. For example, the evaluating includes quantitative or qualitative assessment of SIRT1 mRNA or cDNA levels or evaluating the identity of at least one nucleotide in the SIRT1 molecule.

In another embodiment, the SIRT1 molecule is SIRT1 protein. For example, the evaluating includes quantitative or qualitative assessment of SIRT1 protein levels or determining the identity of at least one amino acid in the SIRT1 protein.

The evaluating can include includes evaluating a cell of the adipose lineage or of the keratinocyte lineage, or a neuronal cell, e.g., a neuron. For example, the evaluating includes evaluating a preadipocyte or adipocyte, e.g., a WAT cell or a BAT cell. The cell of the keratinocyte lineage can be a keratinocyte or a pre-keratinocyte.

The metabolic information can include information about a biometric parameter (such as weight, height, girth or other linear measurement, body mass index, subcutaneous fat content, or visceral fat content); or information about a hormone or a metabolite. For example, information about a hormone includes information about leptin, insulin, adiponectin, or resistin. The information can indicate the concentration of the hormone in a subject. For example, information about a metabolite includes information about triglycerides, fatty acids, LDL particles, HDL particles, or cholesterol. The information can indicate the concentration of the metabolite, e.g., concentration of LDL or HDL particles, in a subject, or a ratio between metabolites or particle size, e.g. LDL and HDL particles. The method can also be adapted for other sirtuins, e.g., human SIRT2, SIRT3, SIRT4, SIRT5, SIRT6, or SIRT7.

In another aspect, the disclosure features a method that includes: monitoring a parameter associated with a SIRT1 molecule from a subject; and providing a therapy to ameliorate a metabolic condition to the subject. The monitoring can occur, before, during, or after the provision of the therapy. The method can be used to evaluate a subject who is being treated for a metabolic condition. A related method can include: treating a subject with a regimen for altering a metabolic condition; before, during, or after the regimen, monitoring a parameter associated with a SIRT1 molecule from the subject; and comparing results of the evaluation to reference information to provide an assessment of the subject.

For example, the regimen or therapy includes a diet, insulin treatment, an exercise regimen, hormone therapy, or administering a pharmaceutical composition. In one embodiment, the assessment is expressed as a risk/propensity for a metabolic disorder, e.g., obesity.

The reference information can be obtained by a corresponding evaluation of a non-obese individual, e.g., a non-obese adult. The reference information can be obtained from the subject prior to the treating.

In one embodiment, the subject is a human adult, e.g., between ages 20-100, or 20-80 or 40-70. In another embodiment, the subject is a juvenile, e.g., a human less than 18, 15, 12, 10, 7, or 5 years of age.

The monitoring can includes evaluating a parameter associated with a SIRT1 molecule from a subject at at least two instances separated by at least 12, 24, 48, 96, or 200 hours. The method can also be adapted for other sirtuins, e.g., human SIRT2, SIRT3, SIRT4, SIRT5, SIRT6, or SIRT7.

In another aspect, the disclosure features a method of evaluating a subject. The method includes evaluating a SIRT1 molecule from a cell of the adipocyte lineage or the keratinocyte lineage; and comparing results of the evaluating to reference information.

For example, the cell is a preadipocyte or adipocyte, e.g., WAT cells or BAT cells. In another example the cell is a prekeratinocyte or a keratinocyte.

For example, the cell is obtained from a human subject. The subject can be identified as having or being at risk for, obesity, an obesity-related disorder, a body mass index in a particular range, or lipodystrophy.

The reference information can includes results of a corresponding evaluation of a SIRT1 molecule from a corresponding cell from a control subject. The comparing can include determining whether the level of SIRT1 expression is at least 1.2, 1.5, 2, 5, or 10 fold different than a reference level evaluated by a corresponding method for a non-obese adult. The evaluating can include evaluating expression of a plurality nucleic acid species to obtain a profile/fingerprint that includes information about SIRT1 expression and at least one additional gene. The comparing can further include comparing expression levels of at least one gene in addition to SIRT1, the comparison being between the subject and corresponding reference information for the additional gene.

The evaluating can include hybridizing a probe (e.g., to mRNA, cDNA), sequence specific amplification or primer extension, nucleic acid sequencing, mass spectroscopy, in situ hybridization, a Northern, subtractive hybridization, or SAGE.

The method can also be adapted for other sirtuins, e.g., human SIRT2, SIRT3, SIRT4, SIRT5, SIRT6, or SIRT7.

In another aspect, the disclosure features a method that includes: providing a cell of a cell of the adipocyte lineage (e.g., pre-adipocyte or an adipocyte cell, e.g., a WAT cell or a BAT cell) or a cell of the keratinocyte lineage; and evaluating expression or activity of a SIRT1 gene in the cell. In one embodiment, fewer than 100, 50, 10, or 5 different genes are evaluated in parallel with SIRT1.

The method can further include contacting the cell with an agent that alters SIRT1 expression or activity prior to the evaluating or with a test agent of unknown function.

The step of evaluating can include one or more of evaluating SIRT1 mRNA levels; evaluating SIRT1 protein levels; evaluating SIRT1 enzymatic activity (e.g., deacetylase activity); evaluating SIRT1 interaction with a SIRT1 binding partner; evaluating SIRT1 interaction with a regulatory DNA sequence (e.g., using a chromatin immunoprecipitation); evaluating expression of a gene regulated by PPAR, e.g., PPAR-gamma, PPAR-delta, or PPAR-alpha (e.g., evaluating PPAR-gamma expression or activity or evaluating UCP1 expression or activity).

In another aspect, the disclosure features a method that includes: identifying a subject has having obesity, being at risk for obesity using clinical criteria, or being overweight; obtaining a sample of cells from the subject; and evaluating expression of a SIRT1 gene in cells of the sample.

In another aspect, the disclosure features a method that includes: evaluating the identity of one or more nucleotides of a sirtuin gene (e.g., a SIRT1 gene) from a subject, thereby providing a first sequence; providing a reference sequence consisting of one or more nucleotides of a sirtuin gene (e.g., a SIRT1 gene) from a reference subject who is indicated for obesity or a body mass index that is at least within the 25^{th} percentile above or below normal; and comparing the first sequence to the reference sequence. The method can further include making a medical, financial, or familial decision as a function of the comparison.

In another aspect, the disclosure features a method that includes: identifying a plurality of human individuals characterized as being underweight, overweight, obese or as having a body mass index in a particular range; comparing distribution of one or more SIRT1 gene polymorphisms among individuals of the plurality.

In another aspect, the disclosure features a method that includes: identifying a subject as having obesity, being at risk for obesity using clinical criteria, or being overweight; and administering an effective amount of an agent that modulates, e.g., increases, SIRT1 activity to the subject. For example, the agent increases SIRT1 mRNA or protein levels in the subject. In one embodiment, the agent increases SIRT1 mRNA or protein levels or SIRT1 enzymatic activity in a cell of the adipocyte lineage, e.g., pre-adipocytes or adipocytes of the subject.

In one embodiment, the agent includes a nucleic acid that includes a sequence encoding SIRT1 or a SIRT1 core domain and an operably linked promoter, or a complement of such a nucleic acid. In another embodiment, the agent includes a polypeptide that contains a SIRT1 core domain. In still another embodiment, the agent is NAD or an NAD precursor or derivative, e.g., an iso-nicotinamide. See, e.g., Biochemistry. Aug. 12, 2003;42(31):9249-56. The agent can also be a compound of Formula I, II, III, or IV.

The agent can be administered in an amount that is also effective for inhibiting pre-adipocyte differentiation, that effective for promoting fat mobilization in WAT cells, that effective for promoting fat burning in BAT cells, and/or that is effective to increase leptin secretion.

The method can further include monitoring a parameter associated with SIRT1, a metabolite, or a hormone, prior to, during or after the administering.

In another aspect, the disclosure provides a method of ameliorating at least one symptom due to a diabetic disorder or insulin resistance, or treating or preventing a diabetic disorder. The method includes administering to a subject an amount of a SIRT1 activator. The amount can be effective to ameliorate the symptom, or treat or prevent the diabetic disorder. For example, the amount can be effective to decrease insulin resistance and increase insulin sensitivity, e.g., to a detectable degree, or at least 0.5, 2, 4, or 5 fold. The amount may be effective to increase fat mobilization and/or burning of fat. For example, the SIRT1 activator is a polyphenol, e.g., a trans-stilbene, e.g., a compound of formula I, II, III, or IV. Exemplary compounds include resveratrol (3,5,4′-trihydroxy-tans-stilbene), butein (3,4,2′,4′-tetrahydroxychalcone); piceatannol (3,5,3′,4′-tetrahydroxy-trans-stilbene); isoliquiritigenin (4,2′,4′-trihydroxychalcone); fisetin (3,7,3′,4′-tetrahydroxyflavone); and quercetin (3,5,7,3′,4′-pentahydroxyflavone). For example, the subject has Type II diabetes. The method can further include increasing β-adrenergic activity, e.g., providing adrenalin or an agent or therapeutic activity that increases adrenalin in the subject.

In another aspect, the disclosure provides a method of ameliorating at least one symptom due to a disorder related to gastric motility, e.g., treating postoperative ileus. For example, the method can be used to increase movement along the digestive tract, e.g., in the intestine. The method includes administering to a subject an amount of a SIRT1 modulator, e.g., activator, for example, the activator can be delivered to the stomach or intestine, e.g., using a suppository, ingestion, or other method. The amount can be effective to increase gastric motility or ameliorate a post-operative condition. For example, the SIRT1 activator is a polyphenol, e.g., a trans-stilbene, e.g., a compound of formula I, II, III, or IV. Exemplary compounds include resveratrol (3,5,4′-trihydroxy-tans-stilbene), butein (3,4,2′,4′-tetrahydroxychalcone); piceatannol (3,5,3′,4′-tetrahydroxy-trans-stilbene); isoliquiritigenin (4,2′,4′-trihydroxychalcone); fisetin (3,7,3′,4′-tetrahydroxyflavone); and quercetin (3,5,7,3′,4′-pentahydroxyflavone). For example, the subject has Type II diabetes. The method can further include increasing P-adrenergic activity, e.g., providing adrenalin or an agent or therapeutic activity that increases adrenalin in the subject.

In another aspect, the disclosure provides a method of increasing fat or lipid metabolism in a subject. The method includes administering to a subject an amount of a SIRT1 activator, e.g., in an amount effective to increase mobilization of fat to the blood from WAT cells and/or to increase fat burning by BAT cells. For example, the amount can be effective to increase the amount of fat metabolism by at least 0.5, 2, 4, or 5 fold.

The method can include, e.g., prior to the administering, identifying a subject as being in need of increased fat or lipid metabolism, e.g., by weighing the subject, determining the BMI of the subject, or evaluating fat content of the subject or SIRT1 activity in cells of the subject. The method can also include monitoring the subject, e.g., during or after the administering. The administering can include one or more dosages, e.g., delivered in boluses or continuously.

In another aspect, the disclosure provides a method of decreasing fat or lipid metabolism in a subject. The method includes administering to a subject an amount of a SIRT1 inhibitor, e.g., in an amount effective to decrease mobilization of fat to the blood from WAT cells and/or to decrease fat burning by BAT cells. For example, the amount can be effective to decrease the amount of fat metabolism by at least 0.5, 2, 4, or 5 fold.

The method can include, e.g., prior to the administering, identifying a subject as being in need of decreased fat or lipid metabolism, e.g., by weighing the subject, determining the BMI of the subject, or evaluating fat content of the subject or SIRT1 activity in cells of the subject. The method can also include monitoring the subject, e.g., during or after the administering. The administering can include one or more dosages, e.g., delivered in boluses or continuously. Monitoring can include evaluating a hormone or a metabolite. Exemplary hormones include leptin, adiponectin, resistin, and insulin. Exemplary metabolites include triglyercides, cholesterol, and fatty acids.

In another aspect, the disclosure provides a method of ameliorating at least one symptom due to a inflammation, or treating or preventing an inflammatory disorder. The method includes administering to a subject an amount of a SIRT1 activator. The amount can be effective to ameliorate the symptom, or treat or prevent inflammation or inflammatory disorder. For example, the SIRT1 activator is a polyphenol, e.g., a trans-stilbene, e.g., a compound of formula I, II, III, or IV. Exemplary compounds include resveratrol (3,5,4′-trihydroxy-tans-stilbene), butein (3,4,2′,4′-tetrahydroxychalcone); piceatannol (3,5,3′,4′-tetrahydroxy-trans-stilbene); isoliquiritigenin (4,2′,4′-trihydroxychalcone); fisetin (3,7,3′,4′-tetrahydroxyflavone); and quercetin (3,5,7,3′,4′-pentahydroxyflavone). For example, the subject has rheumatoid arthritis, lupus, restenosis, psoriasis, graft v. host response, or multiple sclerosis.

In another aspect, the disclosure provides a method of modulating production of a secreted factor, e.g., leptin, adiponectin, or resistin production, in cells of a subject, e.g., in adipocytes of a subject. The method includes administering to a subject an amount of a SIRT1 modulator, e.g., a SIRT1 activator or inhibotor. The amount can be effective to modulate secretion of the secreted factor, e.g., by an increase of at least 1.5, 2, 5, or 10 fold or a decrease of at least 10, 20, 30, 40, 50, 70, or 80%. For example, the SIRT1 activator is a polyphenol, e.g., a trans-stilbene, e.g., a compound of formula I, II, III, or IV. Exemplary compounds include resveratrol (3,5,4′-trihydroxy-tans-stilbene), butein (3,4,2′,4′-tetrahydroxychalcone); piceatannol (3,5,3′,4′-tetrahydroxy-trans-stilbene); isoliquiritigenin (4,2′,4′-trihydroxychalcone); fisetin (3,7,3′,4′-tetrahydroxyflavone); and quercetin (3,5,7,3′,4′-pentahydroxyflavone).

In another aspect, the disclosure provides a method of modulating activity of an adipocyte transcription factor, e.g., a PPAR, e.g., PPAR-gamma, PPAR-delta, or PPAR-alpha, in cells of a subject, e.g., in adipocytes of a subject. The method includes administering to a subject an amount of a SIRT1 modulator, e.g., a SIRT1 activator or inhibotor. The amount can be effective to modulate transcriptional function, e.g., by an increase of at least 1.5, 2, 5, or 10 fold or a decrease of at least 10, 20, 30, 40, 50, 70, or 80%. Exemplary SIRT1 activators and inhibitors are described herein.

In another aspect, the disclosure features a method that includes: identifying a subject as being underweight, at risk for weight loss or cachexia using clinical criteria, or being cachexic; and administering an effective amount of an agent that modulates, e.g., decreases, SIRT1 activity to the subject.

In one embodiment, the agent decreases SIRT1 mRNA or protein levels in the subject. In another embodiment, the agent decreases SIRT1 mRNA or protein levels in pre-adipocytes or adipocytes of the subject. For example, the agent decreases SIRT1 enzymatic activity in pre-adipocytes or adipocytes of the subject.

Exemplary agents include a dsRNA or siRNA that includes a sequence of at least 19 nucleotides that is complementary to a sequence encoding SIRT1; a polypeptide that competes with a SIRT1 substrate for interaction with SIRT1; an antibody or antibody fragment that binds to SIRT1; nicotinamide or vitamin b3; an agent other than nicotinamide and vitamin b3; or a compound of Formula V or VI.

The method can further include monitoring a parameter associated with SIRT1, a metabolite, or a hormone during or after the administering.

In another aspect, the disclosure features a method that includes: providing a compound that interacts with SIRT1 or that modulates SIRT1 activity; contacting the compound to a cell of the adipocyte lineage; and evaluating the cell.

In one embodiment, the evaluating includes evaluating expression of a gene regulated by an adipocyte transcription factor, e.g., a PPAR transcription factor (e.g., PPAR-gamma, PPAR-delta, or PPAR-alpha), PGC1, or a C/EBP transcription factor.

In one embodiment, the cell includes a reporter gene regulated by the adipocyte transcription factor and the evaluating includes evaluating the reporter gene.

In one embodiment, the evaluating includes evaluating the differentiation state of the cell or secretion of a hormone (e.g., leptin, resistin, or adiponectin) by the cell.

In one embodiment, the evaluating includes evaluating fat mobilization by the cell, evaluating fat burning by the cell, or evaluating association of SIRT1 and genomic nucleic acid in the cell. For example, evaluating the association includes crosslinking proteins to nucleic acid, immunoprecipitating SIRT1 or a fragment thereof, and evaluating the nucleic acid associated with SIRT1 immunoprecipitates.

In one embodiment, the step of providing a compound that interacts with SIRT1 or that modulates SIRT1 includes contacting the compound to SIRT1 or a fragment thereof in vitro.

In another aspect, the disclosure features a method that includes: contacting the compound to a preadipocyte cell; and evaluating a parameter associated with a SIRT1 molecule of the cell. A related method includes contacting the compound to a cell of the adipocyte lineage, a cell of the keratinocyte lineage or a neuronal cell. The method can include comparing the parameter to a reference parameter, e.g., for a corresponding cell to which the compound has not been contacted. A difference in the parameter and a reference parameter indicates that the compound alters SIRT1 activity in the preadipocyte.

The method can include evaluating the differentiation state of the predadipocyte cell, or evaluating lipid or fat of the preadipocyte cell. For example, the evaluating includes fractionating cell contents or an optical evaluation.

In another aspect, the disclosure features a method that includes: contacting the compound to an organism (e.g., a mouse, a canine, a pig, or a human); and evaluating a parameter associated with a SIRT1 molecule of a cell of the adipocyte or keratinocyte lineage, from the organism, wherein a difference in the parameter between the parameter and a reference parameter indicates that the compound modulates SIRT1 activity in a cell of the organism.

Another method includes: contacting the compound to a SIRT1 protein in vitro; evaluating an interaction between the compound and the protein; contacting the compound to a cell or organism; and evaluating a differentiation state of the cell or a metabolic parameter of the organism. In one embodiment, evaluating the interaction includes evaluating catalytic activity of the protein in the presence of the compound.

In another aspect, the disclosure features a method that includes: providing a library of compound (e.g., small molecule compounds). For each compound of a plurality of compounds from the library, the method includes: contacting the compound to a SIRT1 protein in vitro; evaluating an interaction between the compound and the SIRT1 protein; if the compound interacts with the SIRT1 protein, contacting the compound to a cell or organism; and evaluating a differentiation state or a metabolic parameter of the cell or organism. For example, the cell includes a reporter gene and/or other combination of heterologous nucleic acids described herein.

In another aspect, the disclosure features a method that includes: providing a library of compound (e.g., small molecule compounds). For each compound of a plurality of compounds from the library, the method includes evaluating the compound using a method described herein.

In another aspect, the disclosure features a method of maturing a lead compound. The method includes: providing a plurality of derivatives/variants of a compound that detectably interacts with a SIRT1 protein; contacting each compound of the plurality to a cell or organism; and evaluating a differentiation state or a metabolic parameter of the cell or organism. The method can further include recording SAR data that associates results of the evaluating and structural information about each compound of the plurality; and/or modeling an interaction between a three-dimensional structural model of a SIRT1 m protein or region thereof and a compound of the plurality.

In another aspect, the invention provides a method of evaluating a test compound or a member of a library of test compounds. The method includes contacting the test compound to a SIRT1 protein or fragment thereof and NCoR or fragment thereof, and evaluating ability of the SIRT1 protein or fragment to interact with the NCorR protein or fragment. In one embodiment, the SIRT1 protein or fragment is first contacted with the NCoR protein or fragment to provide a complex and the test compound is contacted to the complex. In another embodiment, all three proteins (e.g., an any other component of interest, if desired) are added at the same time, or in still other embodiments, the test compound is first contacted to the SIRT1 protein or fragment thereof or the NCoR protein or fragment thereof.

For example, a test compound that reduces interaction between SIRT1 and NCoR can be indicated as an inhibitor of SIRT1 activity or an agent that reduces fat mobilization. A test compound that increases interaction between SIRT1 and NCoR can be indicated as an activator of SIRT1 activity or an agent that increases fat mobilization. In one embodiment, the contacting is effected in vitro, e.g., in a biochemical system, e.g., using purified or partially purified components. For example, any biochemical interaction assay can be used to evaluate interaction between the SIRT1 protein or fragment and the NCorR protein or fragment. Similar methods can be implemented using SMRT or a fragment thereof that interacts with SIRT1.

In another embodiment, the contacting is effected in a cell, e.g., a yeast cell (e.g., using a two hybrid system) or mammalian cell, e.g., a tissue culture cells, e.g., a cultured fibroblast, pre-adipocyte, or adipocyte, e.g., a WAT or BAT cell. Typically the cultured cell includes one or more heterologous nucleic acids for expressing the SIRT1 protein or fragment and/or the NCoR protein or fragment.

Exemplary SIRT1 fragments include about amino acids 10-190, 1-214, 214-541, and 541-747. Exemplary NCoR fragments include 1-751, 92-393, and 1460-1944.

In one embodiment, the compound is a polyphenol, e.g., a trans-stilbene, e.g., resveratrol or other compound of Formula I, II, III, or IV.

In another aspect, the invention features a database that includes a plurality of records, e.g., the records include (1) information about a test compound (e.g., an identifier), (2) information about ability of the test compound to modulate SIRT1 or other sirtuin, and (3) information about ability of the test compound to modulate a parameter of an adipose cell, e.g., a WAT or BAT cell, or transcription of a gene regulated by NCoR.

The method can also be implemented using other members of the SIR2 family, e.g., human SIRT2, 3, 4, 5, 6, or 7.

In another aspect, the invention provides a method of evaluating a test compound or a member of a library of test compounds. The method includes contacting the test compound to a SIRT1 protein or fragment thereof and PGC1 or fragment thereof, and evaluating ability of the SIRT1 protein or fragment to interact with the PGC1 protein or fragment. In one embodiment, the SIRT1 protein or fragment is first contacted with the PGC1 protein or fragment to provide a complex and the test compound is contacted to the complex. In another embodiment, all three proteins (e.g., an any other component of interest, if desired) are added at the same time, or in still other embodiments, the test compound is first contacted to the SIRT1 protein or fragment thereof or the PGC1 protein or fragment thereof.

For example, a test compound that reduces interaction between SIRT1 and PGC1 can be indicated as an inhibitor of SIRT1 activity or an agent that reduces fat burning. A test compound that increases interaction between SIRT1 and PGC1 can be indicated as an activator of SIRT1 activity or an agent that increases fat burning. In one embodiment, the contacting is effected in vitro, e.g., in a biochemical system, e.g., using purified or partially purified components. For example, any biochemical interaction assay can be used to evaluate interaction between the SIRT1 protein or fragment and the PGC1 protein or fragment. In another embodiment, the contacting is effected in a cell, e.g., a yeast cell (e.g., using a two hybrid system) or mammalian cell, e.g., a tissue culture cells, e.g., a cultured fibroblast, pre-adipocyte, or adipocyte. Typically the cultured cell includes one or more heterologous nucleic acids for expressing the SIRT1 protein or fragment and/or the PGC1 protein or fragment.

Exemplary SIRT1 fragments include about amino acids 10-190, 1-214, 214-541, and 541-747.

In one embodiment, the compound is a polyphenol, e.g., a trans-stilbene, e.g., resveratrol or other compound of Formula I, II, III, or IV.

In another aspect, the invention features a database that includes a plurality of records, e.g., the records include (1) information about a test compound (e.g., an identifier), (2) information about ability of the test compound to modulate SIRT1 or other sirtuin, and (3) information about ability of the test compound to modulate a parameter of an adipose cell, e.g., a WAT or BAT cell, or transcription of a gene regulated by PGC1.

The method can also be implemented using other members of the SIR2 family, e.g., human SIRT2, 3, 4, 5, 6, or 7.

In another aspect, the disclosure features a cultured mammalian cell that contains a heterologous reporter gene including a SIRT1 regulatory sequence operably linked to a sequence encoding a detectable protein other than SIRT1. For example, the detectable protein has an enzymatic activity (e.g., β-gal, CAT, ADH, luciferase, etc.). In one embodiment, the cultured cell is a cell other than an adipocytes, but that can differentiate into an adipocyte. In another embodiment, the cultured cell is a pre-adipocyte or a fibroblast. In one embodiment, the cultured cell is an adipocyte.

For example, the detectable protein can fluoresce, e.g., GFP, a variant thereof, etc. In one embodiment, the cultured cell further includes a second reporter gene. For example, the second reporter gene is operably linked to a regulatory sequence of a gene encoding a protein produced specifically by an adipocytes, e.g., leptin.

In another aspect, the disclosure features a transgenic animal having at least one cell that contains a heterologous reporter gene including a SIRT1 regulatory sequence operably linked to a sequence encoding a detectable protein other than SIRT1.

In another aspect, the disclosure features a method of evaluating a compound, the method including: contacting the compound to a cell described herein that includes a heterologous reporter, e.g., for a gene regulated by SIRT1, e.g. a gene described herein regulated by SIRT1; and evaluating expression of the heterologous reporter.

The method can further include evaluating a metabolite in the cell, e.g., lipid or fat (e.g., triglycerides) of the cell.

In another aspect, the disclosure features a preparation that includes: a population of cells of the adipocyte lineage or an extract thereof; and a probe that is specific to a SIRT1 molecule. In one embodiment, the cells include adipocytes or preadipocytes (e.g., a substantially pure population or a population at least 25, 30, 40, 50, 60, 70, 80, 90, or 95% pure). For example, the cells include BAT or WAT cells. The probe can be a nucleic acid probe that is complementary to a SIRT1 nucleic acid; an antibody or fragment thereof that specifically binds to SIRT1; or an acetylated substrate that can be deacetylated by a SIRT1 protein. Similar probes for other sirtuins can also be used. The cells may be lysed, processed or intact.

In another aspect, the disclosure features a preparation that includes: a population of cells of the keratinocyte lineage or an extract thereof; and a probe that is specific to a SIRT1 molecule. In one embodiment, the cells include keratinocytes or prekeratinocytes (e.g., a substantially pure population or a population at least 25, 30, 40, 50, 60, 70, 80, 90, or 95% pure). The probe can be a nucleic acid probe that is complementary to a SIRT1 nucleic acid; an antibody or fragment thereof that specifically binds to SIRT1; or an acetylated substrate that can be deacetylated by a SIRT1 protein. The cells may be lysed, processed or intact. Similar probes for other sirtuins can also be used. Similar preparations of neuronal cells can also be made.

In another aspect, the disclosure features a mammalian cell of the adipocyte or keratinocyte lineage (e.g., an adipocyte or pre-adipocyte cell or a keratinocyte or keratinocyte cell) that contains a dsRNA (e.g., siRNA) that is specific to SIRT1 (or other sirtuin) in an amount effective to alter sirtuin activity in the cell. For example, the mammalian adipocyte or pre-adipocyte cell is cultured. The mammalian cell may also be in a pharmaceutical composition or other form for administration to a subject.

In another aspect, the disclosure features a mammalian cell of the adipocyte or keratinocyte lineage (e.g., an adipocyte or pre-adipocyte cell or a keratinocyte or keratinocyte cell) that contains a heterologous nucleic acid that includes a sequence encoding a polypeptide that includes a SIRT1 core domain and an operably linked promoter, wherein activation of the promoter can produce the polypeptide in an amount sufficient to alter SIRT1 activity in the cell. For example, the mammalian adipocyte or pre-adipocyte cell is cultured.

In another aspect, the disclosure features a purified complex including SIRT1 or an PGC1 interacting fragment thereof, and (ii) PGC1 or a SIRT1 interacting fragment thereof. The complex can further include (iii) a PPAR protein, e.g., PPAR-gamma, PPAR-delta, or PPAR-alpha, or fragment thereof. The complex can be at least 10, 20, 40, 50, 60, 70, 80, 90, or 95% pure. The disclosure also features an antibody or other protein ligand that specifically recognizes the complex, but does not substantially bind to any of the complex components in isolation.

In another aspect, the disclosure features a purified complex including SIRT1 or an NCoR or SMRT interacting fragment thereof, and (ii) NCoR, or SMRT, or a SIRT1 interacting fragment thereof. The complex can further include (iii) a PPAR protein, e.g., PPAR-gamma, PPAR-delta, or PPAR-alpha, or fragment thereof. The complex can be at least 10, 20, 40, 50, 60, 70, 80, 90, or 95% pure. The disclosure also features an antibody or other protein ligand that specifically recognizes the complex, but does not substantially bind to any of the complex components in isolation.

In another aspect, the disclosure features a method that includes: providing a cell of the adipose lineage or of the keratinocyte lineage, or a neuronal cell, e.g., a neuron; and modulating SIRT1 activity in the cell. For example, the cell can be a preadipocyte or adipocyte, e.g., a WAT cell or a BAT cell. The cell of the keratinocyte lineage can be a keratinocyte or a pre-keratinocyte.

In one embodiment, the modulating includes increasing SIRT1 activity, e.g., using a SIRT1 activator.

In another embodiment, the modulating includes decreasing SIRT1 activity, e.g., using a SIRT inhibitor.

In one embodiment, the modulating includes contacting the cell with a dsRNA (e.g., an siRNA).

In one embodiment, the modulating includes introducing a nucleic acid that includes a sequence that encodes a polypeptide including a SIRT1 core domain or a sequence complementary to a SIRT1 coding sequence, e.g., thereby providing a SIRT1 activity to the cell. The method can also be implemented with other sirtuins, e.g., human SIRT2, SIRT3, SIRT4, SIRT5, SIRT6, or SIRT7.

In another aspect, the disclosure features a method that includes: providing a mammalian cell; modulating sirtuin activity (e.g., SIRT1) in the cell; and evaluating a lipid or fat-associated parameter of the cell. For example, the evaluating includes an optical evaluation of the cell.

In another aspect, the disclosure features a method that includes: providing a mammalian cell; modulating sirtuin activity (e.g., SIRT1) in the cell; and evaluating the differentiation state of the cell using an indicator of adipocyte differentiation. For example, the indicator is expression of leptin, adiponectin, or resistin. In another example, the indicator is expression or activity of an adipocyte transcription factor (e.g., a C/EBP protein or a PPAR protein).

In one embodiment, the cell contains a nucleic acid that can express a C/EBP protein (e.g., C/EBPα), e.g., a heterologous nucleic acid, and a reporter nucleic acid that includes a regulatory sequence of gene that is specifically or selectively expressed in adipocytes or a reporter nucleic acid that includes a regulatory sequence of a secrete protein produced by adipocytes. For example, the secreted protein produced by adipocytes is leptin, adiponectin, or resistin. Alternatively, the cell contains a nucleic acid that can express a PPAR protein (e.g., PPAR-gamma, PPAR-delta, or PPAR-alpha), e.g., a heterologous nucleic acid, and a reporter nucleic acid that includes a regulatory sequence that is bound by an AP2 protein.

Some Definitions

An “adipocyte” is a cell that is characterized by: high triglyceride content, expresses ion of adipogenic proteins and transcription factors such as PPARγ or C/EBPα, an ability to produce and secrete proteins such as leptin, and an ability to release fatty acids upon stimulation of the adrenergic pathway.

A “pre-adipocyte” is a cell that has committed to the adipocyte lineage (i.e., has expressed some early pro-adipocyte genes), but does not: (1) have the mature adipocyte phenotype of intracellular triglyceride accumulation, or (2) expresses adipogenic transcription factors, proteins and enzymes.

A “fibroblast” is a pluripotent cell that has no adipocyte or pre-adipocyte characteristic but that can be stimulated to differentiate into one cellular lineage (e.g. muscle or adipocyte) given proper hormonal stimulation.

A “cell of the adipocyte lineage” is a cell in the adipocyte lineage, e.g., an adipocyte (including a brown adipose cell or white adipose cell), a pre-adipocyte, a fibroblast, or other pluripotent cell that can differentiate into an adipocyte. Brown adipose cells are also termed “BAT cells”; white adipose cells are also termed “WAT cells.”

A “keratinocyte” is a skin cell that can express keratin.

A “cell of the keratinocyte lineage” is a cell in the keratinocyte lineage, e.g., an keratinocyte (including a brown adipose cell or white adipose cell), a pre-keratinocyte, a fibroblast, or other pluripotent cell that can differentiate into a keratinocyte.

The term “an aberrant expression” refers to level of expression of that nucleic acid or protein which differs from the level of expression of that nucleic acid or protein in a reference tissue, e.g., a healthy tissue. Expression can refer to transcription of a gene and/or translation of a transcript. For example, expression can be assessed by evaluating mRNA and/or protein levels. A cell can have an aberrant expression level of a gene due to overexpression or underexpression of that gene, e.g., relative to a reference cell, e.g., a cell from a healthy tissue or subject.

The term “agonist”, as used herein, refers to an agent that mimics or upregulates (e.g., increases, potentiates or supplements) an activity of a compound, e.g., a protein. An agonist can be a wild-type protein or derivative thereof having at least one bioactivity of the wild-type protein. An agonist can also be a compound that upregulates expression of a gene or which increases at least one activity of a protein. An agonist can also be a compound which increases the interaction of a polypeptide with another molecule, e.g., a substrate or binding partner.

“Antagonist” as used herein refers to an agent that downregulates (e.g., decreases, suppresses or inhibits) at least one activity of a compound, e.g., a protein. An antagonist can be a compound which inhibits or decreases an activity of a protein or an interaction between a protein and another molecule, e.g., a substrate or binding partner. An antagonist can also be a compound that downregulates expression of a gene or which reduces the amount of expressed protein present.

The term “antibody,” as used herein, refers to a protein that includes at least one immunoglobulin variable domain, or more typically, at least one pair that consists of a immunoglobulin heavy chain variable domain and a light chain variable domain that interact to form an antigen binding site. The term includes whole antibodies, e.g., of any isotype (IgG, IgA, IgM, IgE, etc.), and includes exemplary fragments, e.g., Fab's, scFv's, and Fv fragments. Antibodies can be fragmented using conventional techniques or recombinant nucleic acid engineering. Fragments can be screened for utility in the same manner as described above for whole antibodies. As used herein, the term includes polyclonal, monoclonal, monospecific, or other purified preparations of antibodies and recombinant antibodies. Techniques for preparing monoclonal and polyclonal antibodies are well known in the art. Campbell, “Monoclonal Antibody Technology: Laboratory Techniques in Biochemistry and Molecular Biology”, Elsevier Science Publishers, Amsterdam, The Netherlands (1984); and St. Groh et al., J. Immunol. Methods, 35, pp. 1-21 (1980).

As used herein the term “bioactive fragment of a polypeptide” refers to a fragment of a full-length polypeptide, wherein the fragment has at least one detectable function. For example, a bioactive fragment may specifically agonize (mimic) or antagonize (inhibit) the activity of a wild-type polypeptide. The bioactive fragment preferably is a fragment capable of interacting with at least one other molecule, e.g., a co-factor protein, small molecule, or DNA, which a full length protein can bind. Exemplary bioactive fragments of SIRT1 include fragments that have deacetylase activity (e.g., 214-541) and fragments that can interact with NcoR (e.g., 10-190 or 1-214).

“Biological activities” include catalyzing a reaction (e.g., a deacetylation), binding to polypeptides, binding to other proteins or molecules, activity as a DNA binding protein, as a transcription regulator, ability to bind damaged DNA, etc.

The term “biomarker” refers a biological molecule, e.g., a nucleic acid, peptide, hormone, etc., whose presence or concentration can be detected and correlated with a known condition, such as a disease state.

“Host cells”, or “recombinant host cells”, are used interchangeably herein. A reference to a “cell” can include not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. In certain implementations, cells of the adipocyte lineage (e.g., preadipocytes or adipocytes) can be used as host cells.

Cells used in any method described herein can be, for example, mammalian cells, e.g., human cells. Cell can be tissue culture cells, e.g., including primary culture cells and transformed cells.

A “delivery complex” means a molecule that results in higher affinity binding of a nucleic acid, protein, polypeptide or peptide to a target cell surface and/or increased cellular or nuclear uptake by a target cell. Examples of targeting means include: sterols (e.g., cholesterol), lipids (e.g., a cationic lipid, virosome or liposome), viruses (e.g., adenovirus, adeno-associated virus, and retrovirus), or target cell-specific binding agents (e.g., ligands recognized by target cell specific receptors).

The term “DNA sequence encoding a polypeptide” refers to any nucleic acid that encodes a polypeptide, including, e.g., a cDNA, a cDNA fragment, a genomic DNA, a genomic DNA fragment, and a synthetic DNA. Moreover, certain differences in nucleotide sequences may exist between individual organisms, of the same or different species, which are called alleles. Such allelic differences may or may not result in differences in amino acid sequence of the encoded polypeptide yet still encode a polypeptide with the same biological activity.

As used herein, the terms “gene”, “recombinant gene”, and “gene construct” refer to a nucleic acid associated with an open reading frame, e.g., including one or exons and (optionally) intron sequences and (optionally) regulatory sequences. A “recombinant gene” refers to nucleic acid encoding a polypeptide and comprising exon sequences, though it may optionally include intron sequences which are derived from, for example, a related or unrelated chromosomal gene. A gene can further include regulatory sequences, e.g., transcriptionally regulatory sequences (e.g., a promoter, enhancer) and translational regulatory sequences (e.g., 5′ and 3′ untranslated regions) and so forth.

“Homology”, “homologs of”, “homologous”, or “identity” or “similarity” refers to sequence similarity between two polypeptides or between two nucleic acid molecules, with identity being a more strict comparison. Homology and identity can each be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e., structurally related, at positions shared by the amino acid sequences. An “unrelated” or “non-homologous” sequence shares less than 40% identity, though preferably less than 25% identity, with the reference sequence.

Hypertrophic growth is defined as an increase in adipocyte size that is stimulated by lipid accumulation. Hyperplastic growth is defined as an increase in the number of cells, e.g., adipocytes in adipose tissue and is thought to occur primarily by mitosis of pre-existing adipocytes that have reached critical levels of lipid accumulation and size.

“Obesity” refers to a condition in which a subject has a body mass index of greater than or equal to 30. “Over-weight” refers to a condition in which a subject has a body mass index of greater or equal to 25.0. The body mass index and other definitions are according to the “NIH Clinical Guidelines on the Identification and Evaluation, and Treatment of Overweight and Obesity in Adults” (1998). In particular, obesity can lead to type II diabetes in successive phases. Clinically, these phases can be characterized as normal glucose tolerance, impaired glucose tolerance, hyperinsulinemic diabetes, and hypoinsulinemic diabetes. Such a progressive impairment of glucose storage correlates with a rise in basal glycemia.

“Obesity-related disease” and “Fat-related metabolic disorder” include, but are not limited to, anorexia nervosa, wasting, AIDS-related weight loss, bulimia, cachexia, lipid disorders including hyperlipidemia and hyperuricemia, insulin resistance, noninsulin dependent diabetes mellitus (NIDDM, or Type II diabetes), insulin dependent diabetes mellitus (IDDM or Type I diabetes), diabetes-related complications including microangiopathic lesions, ocular lesions, retinopathy, neuropathy, and renal lesions, cardiovascular disease (including cardiac insufficiency, coronary insufficiency, and high blood pressure), atherosclerosis, atheromatous disease, stroke, hypertension, Syndrome X, gallbladder disease, osteoarthritis, sleep apnea, forms of cancer such as uterine, breast, colorectal, kidney, and gallbladder, high cholesterol levels, complications of pregnancy, menstrual irregularities, hirsutism, muscular dystrophy, infertility, a weight-related disorder (characterized by a subject being over or under weight, e.g., being within the top or bottom 25^{th} percentile of body mass index) and increased surgical risk. In preferred embodiments, a treated or diagnosed subject is a mammal, preferably a human.

Fat-related metabolic disorders include disorders in which (i) increased fat storage, reduced fat mobilization, and/or reduced fat burning is desired, and (ii) other disorders in which reduced fat storage, increased fat mobilization and/or increased fat burning is desired. Examples of the first category of disorders include, e.g., anorexia nervosa, wasting, AIDS-related weight loss, bulimia, cachexia. Examples of the latter category include, e.g., obesity, cardiovascular disease, osteoarthritis. The classification of other disorders (e.g., infertility, increased surgical risk, pregnancy complications) may depend on the weight of the subject, e.g., whether the subject is over- or underweight. Overweight subjects can be treated, e.g., with an agent that increases SIRT1 activity, and underweight subject can be treated, e.g., with an agent that decreases SIRT1 activity.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences. Other techniques for determining sequence identity are well-known and described in the art. Exemplary biopolymers have sequences that are at least 70, 75, 80, 85, 90, 92, 93, 94, 95, 96, 97, 98, 99, or 99.5% identical to a sequence described herein.

The term “interact” as used herein is meant to include detectable interactions (e.g., biochemical interactions) between molecules, such as interaction between protein-protein, protein-nucleic acid, nucleic acid-nucleic acid, and protein-small molecule or nucleic acid-small molecule in nature.

The term “isolated” as used herein with respect to nucleic acids, such as DNA or RNA, refers to molecules separated from other DNAs, or RNAs, respectively that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both partially purified, purified (e.g., at least 10, 30, 70, 80, 90, 95, 98, 99% pure) and recombinant polypeptides.

The term “sirtuin” and “sirtuin” include amino acids sequences that have a SIR2 domain or a fragment thereof (the fragment need not also include the SIR2 domain). The fragments have at least one function of a sirtuin protein or are folded. Functional fragments can, for example, have deacetylase activity or interact with a sirtuin binding partner, e.g., PPAR-gamma, PGC1, or NcoR. A sirtuin can be encoded using a nucleic acid that includes artificially chosen codons. Sirtuins include proteins that are scored as hits in the Pfam family for SIR2 domains.

To identify the presence of a “SIR2” domain in a protein sequence, and make the determination that a polypeptide or protein of interest has a particular profile, the amino acid sequence of the protein can be searched against the Pfam database of HMMs (e.g., the Pfam database, release 2.1) using the default parameters (available from the Sanger web site: www-sanger-ac-uk/Software/Pfam/HMM_search). The SIR2 domain is indexed in Pfam as entry number PF02146 and in INTERPRO as INTERPRO description (entry IPR003000). At present PF02146 includes 168 sequences. SIR2 domains can have a fold that is structurally similar to PDB entry 1ICI or 1M2H.

For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314. For example, closely related homologs of human SIRT1 (NP_{—}036370.2) in M.musculus (NP_{—}062786.1; 737 amino acid) and R. norvegicus (XP_{—}228146.2; 700 amino acid). Additional homologs include B.taurus Bt.13818; C.elegans Cel.12479; C.intestinalis Cin.7948 ; C.intestinalis Cin.13319; D.rerio Dr.10536; D.melanogaster Dm.415; G.gallus Gga.1 1206; M.musculus Mm.150679; M.musculus Mm.348981; M.musculus Mm.348984; R.norvegicus Rn.42098; X.laevis X1.8444; and X.tropicalis Str.10623.

Human sirtuins include, e.g., the following amino acids sequences: human sirtuin 1 (GenBank Accession No: NP_{—}036370.2) (SEQ ID NO: 15); human sirtuin 2 isoform 1 (GenBank Accession No: NP_{—}036369.2) (SEQ ID NO: 16); human sirtuin 2 isoform 2 (GenBank Accession No: NP_{—}085096.1) (SEQ ID NO: 17); human sirtuin 3 (GenBank Accession No: NP_{—}036371.1) (SEQ ID NO: 18); human sirtuin 4 (GenBank Accession No: NP_{—}036372.1) (SEQ ID NO: 19); human sirtuin 5 isoform 1 (GenBank Accession No: NP_{—}036373.1) (SEQ ID NO: 20); human sirtuin 5 isoform 2 (GenBank Accession No: NP_{—}112534.1) (SEQ ID NO: 21); human sirtuin 6 (GenBank Accession No: NP_{—}057623.1) (SEQ ID NO: 22); and human sirtuin 7 (GenBank Accession No: NP_{—}057622.1) (SEQ ID NO: 23). With respect to any embodiment described herein for SIRT1, the embodiment can be adapted and implemented using another sirtuin, e.g., SIRT2, SIRT3, SIRT4, SIRT5, SIRT6, or SIRT7.

The terms “sirtuin nucleic acid” and “mammalian homolog of a SIR2 gene” includes all nucleic acids sequences that encode sirtuin proteins, and all nucleotide sequences that are complementary or fragments thereof that encode functional or folded fragments of a sirtuin protein. Exemplary sirtuin nucleic acids include human Sir2 SIRT1 mRNA (GenBank Accession No. AF083106)(SEQ ID NO: 1); mouse SIRT1 mRNA (GenBank Accession No: AF214646) )(SEQ ID NO: 2); rat SIRT1 mRNA (GenBank Accession No: XM_{—}228146) )(SEQ ID NO: 3); human Sir2 SIRT2 mRNA (GenBank Accession No: AF083107) )(SEQ ID NO: 4); mouse Sir2 SIRT2 mRNA (GenBank Accession No: AF299337) )(SEQ ID NO: 5); human Sir2 SIRT3 mRNA (GenBank Accession No: AF083108) )(SEQ ID NO: 6); mouse Sir2 SIRT3 mRNA splice variants (GenBank Accession No: AF299339 )(SEQ ID NO: 7) and AF 299338 (SEQ ID NO: 8)); human Sir2 SIRT4 mRNA (GenBank Accession No: AF083109)(SEQ ID NO: 9); human Sir2 SIRT5 mRNA (GenBank Accession No: AF083110) (SEQ ID NO: 10); human Sir2 SIRT6 mRNA (GenBank Accession No: AF233396)(SEQ ID NO: 11); mouse Sir2 SIRT6 mRNA (GenBank Accession No: NM_{—}181586) (SEQ ID NO: 12); human Sir2 SIRT7 mRNA (GenBank Accession No: AF233395) (SEQ ID NO: 13); mouse Sir2 SIRT7 mRNA (GenBank Accession No: NM_{—}153056) (SEQ ID NO: 14), as well as all genomic DNA, cDNA, and synthetic DNA sequences that correspond to, or are complementary or are fragments, e.g., encoding functional protein fragments of the aforementioned nucleic acid sequences.

The terms “modulated” and “differentially regulated” as used herein include upregulation (i.e., activation or stimulation (e.g., by agonizing or potentiating)) and downregulation (i.e., inhibition or suppression (e.g., by antagonizing, decreasing or inhibiting)).

As used herein, the term “nucleic acid” refers to polynucleotides such as deoxyribonucleic acid (DNA), and, where appropriate, ribonucleic acid (RNA) and combinations thereof. The term also includes, as equivalents, analogs of RNA or DNA made from nucleotide analogs, and single (sense or antisense) and double-stranded polynucleotides. ESTs, chromosomes, cDNAs, mRNAs, and rRNAs.

The term “polymorphism” refers to the coexistence of more than one form of a gene or portion (e.g., allelic variant) thereof in a population of organisms. A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides long. A “polymorphic gene” refers to a gene having at least one polymorphic region.

The term “allele”, which is used interchangeably herein with “allelic variant”, refers to a nucleotide polymorphism which may be present in a cell, e.g., in a gene or elsewhere on a chromosome. Alleles occupy the same locus or position on homologous chromosomes, but may also be introduced, artificially, at heterologous positions. When a subject has two identical alleles of a gene, the subject is said to be homozygous for that gene or allele. When a subject has two different alleles of a gene, the subject is said to be heterozygous for the gene. The term “wild-type allele” refers to an allele of a gene which is present in at least 40% of the population. There can be several different wild-type alleles of a specific gene. Wild-type alleles may encode an amino acid sequence set forth herein or a natural variant thereof.

As used herein, the term “promoter” means a DNA sequence that regulates expression of a particular DNA sequence operably linked to the promoter, and which effects expression of the particular DNA sequence in cells. The term encompasses “tissue specific” promoters, i.e., promoters which regulate expression of the selected DNA sequence as a function of cellular state, e.g., differentiation state. Typically tissue specific promoters are active only in specific cells (e.g., cells of a specific tissue). The term also covers so-called “leaky” promoters, which regulate expression of a selected DNA primarily in one tissue, but cause expression in other tissues as well. Other promoters that can be used include non-tissue specific promoters and promoters that are constitutively expressed or that are inducible (i.e., expression levels can be controlled).

The terms “protein”, “polypeptide”, and “peptide” are used interchangeably herein to refer to a polymer of amino acids. However, a protein may include more than one polypeptide chain. A polypeptide can be a gene product, although some polypeptides can be produced synthetically.

The term “recombinant protein” refers to a protein which is produced by recombinant DNA techniques. In an exemplary method, DNA encoding a polypeptide is inserted into a suitable expression vector which is in turn used to transform a host cell to produce the heterologous protein. In another exemplary method, homologous recombination is used to insert a heterologous regulatory sequence into an endogenous gene. Moreover, the phrase “derived from,” with respect to a recombinant gene, is meant to include within the meaning of “recombinant protein” those proteins having an amino acid sequence of a native polypeptide, or an amino acid sequence similar thereto which is generated by mutations including substitutions and deletions (including truncation) of a naturally occurring form of the polypeptide.

“Small molecule” as used herein, refers to a composition, which has a molecular weight of less than about 5 kD and most preferably less than about 4 kD. Small molecules can be nucleic acids, peptides, polypeptides, peptidomimetics, carbohydrates, lipids or other organic (carbon-containing) or inorganic molecules. In preferred embodiments, the small molecule has a molecular weight of less than 1500, 1000, 800, 700, or 500 Daltons.

As used herein, the term “specifically hybridizes” or “specifically detects” refers to the ability of a nucleic acid molecule to hybridize to at least a portion of, for example, approximately 6, 12, 15, 20, 30, 50, or 100 contiguous nucleotides of a nucleic acid comprising a mammalian homolog of a SIR2 gene, or a sequence complementary thereto, or naturally occurring mutants thereof, such that it has less than 15%, preferably less than 10%, and more preferably less than 5% background hybridization to a cellular nucleic acid (e.g., mRNA or genomic DNA) encoding a different protein. In preferred embodiments, the oligonucleotide probe detects only a specific nucleic acid, e.g., it does not substantially hybridize to similar or related nucleic acids, or complements thereof. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0. 1% SDS at 65° C.; and 4) very high stringency hybridization conditions are 0.5M sodium phosphate, 7% SDS at 65° C., followed by one or more washes at 0.2×SSC, 1% SDS at 65° C. In certain embodiments, a SIRT1 gene hybridizes to a nucleic acid that encodes the catalytic domain of a SIR2 protein described herein, e.g., human SIRT1.

A “subject” can be an animal, preferably a mammal, and most preferably a human.

“Transcriptional regulatory sequence” refers to DNA sequences, such as initiation signals, enhancers, and promoters, which induce or control transcription of protein coding sequences with which they are operably linked. In preferred embodiments, transcription of one of the genes is under the control of a promoter sequence (or other transcriptional regulatory sequence) which controls the expression of the recombinant gene in a cell-type in which expression is intended.

The term “treating” as used herein encompasses preventing as well as ameliorating at least one symptom of the condition or disease, or providing a prophylaxis or otherwise preventing at least one symptom. Treating can also include rendering one or more physiological functions to a more normal or less pathological state.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In one embodiment, a vector is a plasmid, but a vector can also be in another form, e.g., a linear nucleic acid.

Statistical significance can be determined by any art known method. Exemplary statistical tests include: the Students T-test, Mann Whitney U non-parametric test, and Wilcoxon non-parametric statistical test. Some statistically significant relationships have a P value of less than 0.05, or 0.02. Particular methods may show a difference, e.g., result, that are statistically significant (e.g., P value <0.05 or 0.02).

This disclosure incorporates by reference Picard et al. (2004) Nature doi:10.103/nature02583, Nature 429(6993):771-6. All cited references, patents, and patent applications, inclusive of 60/484,836, filed Jul. 3, 2003, and a PCT Serial Number (to be entered), entitled “SIRT1 MODULATION OF ADIPOGENESIS AND ADIPOSE FUNCTION,” bearing attorney docket number 13407-058WO1, filed Jul. 6, 2004, are incorporated by reference for all purposes.

These and other aspects of the instant inventions are described further in the detailed description.

### BRIEF DESCRIPTION OF THE FIGURES

FIG. 1 depicts results indicating that SIRT1 modulates the amount of fat that can be released upon lipolysis. Lipolysis is reduced when SIRT1 is overexpressed and is increased when SIRT1 is underexpressed.

FIG. 2 depicts the effect of SIRT1 on leptin transcription. SIRT1 represses C/EBPα-induced leptin transcription in a dose-dependent manner.

### DETAILED DESCRIPTION

SIRT1 modulates critical components of adipocyte physiology in mammals. For example, SIRT1 activates fat mobilization in white adipocytes (WAT cells) and can also contribute to the burning of fat in brown adipocytes (BAT cells).

In WAT cells, SIRT1 can modulate expression of genes, e.g. genes regulated by PPAR-gamma. For example, SIRT1 can interact with cofactors NCoR (nuclear receptor co-repressor) and SMRT (silencing mediator or retinoid and thyroid hormone receptors).

SIRT1 also modulates the differentiation of cells in the adipocyte lineage.

In brown fat cells (BAT cells), SIRT1 binds to the UCP1 promoter. The human UCP1 gene is located at about 4q28-q31 on chromosome 4. The UCP1 promoter can include about nucleotides 142052800 to 142058843 on the plus strand of human chromosome 4, these nucleotides correspond to about 3023229 to 3029272 of GENBANK® entry NT_{—}016606.16. Useful promoter nucleic acids can include at least about 500, 800, 1 kb, 2 kb, 3 kb, or 4 kb of the above region, for example, regions of such size that include the mRNA start site, the TATA box, or the UCP1 ATG. For example, the promoter can terminate at the mRNA start site, the TATA box, or the UCP1 ATG. Such regions may include one or more of: a PPAR-gamma binding site, TRE/RARE binding site, NF-E2 binding site, and cAMP responsive elements. See, e.g., Rim et al. (2002) J. Biol. Chem. 277:34589-34600 for exemplary binding sites and exemplary promoter regions.

At the UCP1 promoter, SIRT1 can form a ternary complex with PGC1 and PPAR-gamma. An exemplary amino acid sequences for PGC1 includes:

>gi|7019499|ref|NP_{—}037393.1| peroxisome proliferative activated receptor gamma coactivator 1; ligand effect modulator-6; PPAR gamma coactivator-1 [Homo sapiens]

| | |
|---|---|
| MAWDMCNQDSESVWSDIECAALVGEDQPLCPDLPELDLSELDVNDLDTDSFLGGLKWCS | (SEQ ID NO:32) |
| DQSEIISNQYNNEPSNIFEKIDEENEANLLAVLTETLDSLPVDEDGLPSFDALTDGDVT | |
| TDNEASPSSMPDGTPPPQEAEEPSLLKKLLLAPANTQLSYNECSGLSTQNHANHNHRIR | |
| TNPAIVKTENSWSNKAKSICQQQKPQRRPCSELLKYLTTNDDPPHTKPTENRNSSRDKC | |
| TSKKKSHTQSQSQHLQAKPTTLSLPLTPESPNDPKGSPFENKTIERTLSVELSGTAGLT | |
| PPTTPPHKANQDNPFRASPKLKSSCKTVVPPPSKKPRYSESSGTQGNNSTKKGPEQSEL | |
| YAQLSKSSVLTGGHEERKTKRPSLRLFGDHDYCQSINSKTEILINISQELQDSRQLENK | |
| DVSSDWQGQICSSTDSDQCYLRETLEASKQVSPCSTRKQLQDQEIRAELNKHFGHPSQA | |
| VFDDEADKTGELRDSDFSNEQFSKLPMFINSGLAMDGLFDDSEDESDKLSYPWDGTQSY | |
| SLFNVSPSCSSFNSPCRDSVSPPKSLFSQRPQRMRSRSRSFSRHRSCSRSPYSRSRSRS | |
| PGSRSSSRSCYYYESSHYRHRTHRNSPLYVRSRSRSPYSRRPRYDSYEEYQHERLKREE | |
| YRREYEKRESERAKQRERQRQKAIEERRVIYVGKIRPDTTRTELRDRFEVFGEIEECTV | |
| NLRDDGDSYGFITYRYTCDAFAALENGYTLRRSNETDFELYFCGRKQFFKSNYADLDSN | |
| SDDFDPASTKSKYDSLDFDSLLKEAQRSLRR | |

An exemplary amino acid sequence for PPAR-gamma is as follows:

gi|20336231|ref|NP_{—}005028.3| peroxisome proliferative activated receptor gamma isoform 1; PPAR gamma [Homo sapiens]

| | |
|---|---|
| MVDTEMPFWPTNFGISSVDLSVMEDHSHSFDIKPFTTVDFSSISTPHYEDIPFTRTDPV | (SEQ ID NO:33) |
| VADYKYDLKLQEYQSAIKVEPASPPYYSEKTQLYNKPHEEPSNSLMAIECRVCGDKASG | |
| FHYGVHACEGCKGFFRRTIRLKLIYDRCDLNCRIHKKSRNKCQYCRFQKCLAVGMSHNA | |
| IRFGRMPQAEKEKLLAEISSDIDQLNPESADLRALAKHLYDSYIKSFPLTKAKARAILT | |
| GKTTDKSPFVIYDMNSLMMGEDKIKFKHITPLQEQSKEVAIRIFQGCQFRSVEAVQEIT | |
| EYAKSIPGFVNLDLNDQVTLLKYGVHEIIYTMLASLMNKDGVLISEGQGFMTREFLKSL | |
| RKPFGDFMEPKFEFAVKFNALELDDSDLAIFIAVIILSGDRPGLLNVKPIEDIQDNLLQ | |
| ALELQLKLNHPESSQLFAKLLQKMTDLRQIVTEHVQLLQVIKKTETDMSLHPLLQEIYK | |
| DLY | |

gi|20336229|ref|NP_{—}056953.2| peroxisome proliferative activated receptor gamma isoform 2; PPAR gamma [Homo sapiens]

| | |
|---|---|
| MGETLGDSPIDPESDSFTDTLSANISQEMTMVDTEMPFWPTNFGISSVDLSVMEDHSHS | (SEQ ID NO:34) |
| FDIKPFTTVDFSSISTPHYEDIPFTRTDPVVADYKYDLKLQEYQSAIKVEPASPPYYSE | |
| KTQLYNKPHEEPSNSLMAIECRVCGDKASGFHYGVHACEGCKGFFRRTIRLKLIYDRCD | |
| LNCRIHKKSRNKCQYCRFQKCLAVGMSHNAIRFGRMPQAEKEKLLAEISSDIDQLNPES | |
| ADLRALAKHLYDSYIKSFPLTKAKARAILTGKTTDKSPFVIYDMNSLMMGEDKIKFKHI | |
| TPLQEQSKEVAIRIFQGCQFRSVEAVQEITEYAKSIPGFVNLDLNDQVTLLKYGVHEII | |
| YTMLASLMNKDGVLISEGQGFMTREFLKSLRKPFGDFMEPKFEFAVKFNALELDDSDLA | |
| IFIAVIILSGDRPGLLNVKPIEDIQDNLLQALELQLKLNHPESSQLFAKLLQKMTDLRQ | |
| IVTEHVQLLQVIKKTETDMSLHPLLQEIYKDLY | |

An exemplary sequence of NcoR is:

sp|O75376|NCR1_HUMAN Nuclear receptor corepressor 1 (N—CoR1) (N—CoR)—Homo sapiens (Human).

| | |
|---|---|
| MSSSGYPPNQGAFSTEQSRYPPHSVQYTFPNTRHQQEFAVPDYRSSHLEVSQASQLLQQ | (SEQ ID NO:35) |
| QQQQQLRRRPSLLSEFHPGSDRPQERRTSYEPFHPGPSPVDHDSLESKRPRLEQVSDSH | |
| FQRVSAAVLPLVHPLPEGLRASADAKKDPAFGGKHEAPSSPISGQPCGDDQNASPSKLS | |
| KEELIQSMDRVDREIAKVEQQILKLKKKQQQLEEEAAKPPEPEKPVSPPPVEQKHRSIV | |
| QIIYDENRKKAEEAHKIFEGLGPKVELPLYNQPSDTKVYHENIKTNQVMRKKLILFFKR | |
| RNHARKQREQKICQRYDQLMEAWEKKVDRIENNPRRKAKESKTREYYEKQFPEIRKQRE | |
| QQERFQRVGQRGAGLSATIARSEHEISEIIDGLSEQENNEKQMRQLSVIPPMMFDAEQR | |
| RVKFINMNGLMEDPMKVYKDRQFMNVWTDHEKEIFKDKFIQHPKNFGLIASYLERKSVP | |
| DCVLYYYLTKKNENYKALVRRNYGKRRGRNQQIARPSQEEKVEEKEEDKAEKTEKKEEE | |
| KKDEEEKDEKEDSKENTKEKDKIDGTAEETEEREQATPRGRKTANSQGRRKGRITRSMT | |
| NEAAAASAAAAAATEEPPPPLPPPPEPISTEPVETSRWTEEEMEVAKKGLVEHGRNWAA | |
| IAKMVGTKSEAQCKNFYFNYKRRHNLDNLLQQHKQKTSRKPREERDVSQCESVASTVSA | |
| QEDEDIEASNEEENPEDSEVEAVKPSEDSPENATSRGNTEPAVELEPTTETAPSTSPSL | |
| AVPSTKPAEDESVETQVNDSISAETAEQMDVDQQEHSAEEGSVCDPPPATKADSVDVEV | |
| RVPENHASKVEGDNTKERDLDRASEKVEPRDEDLVVAQQINAQRPEPQSDNDSSATCSA | |
| DEDVDGEPERQRMFPMDSKPSLLNPTGSILVSSPLKPNPLDLPQLQHRAAVIPPMVSCT | |
| PCNIPIGTPVSGYALYQRHIKAMHESALLEEQRQRQEQIDLECRSSTSPCGTSKSPNRE | |
| WEVLQPAPHQVITNLPEGVRLPTTRPTRPPPPLIPSSKTTVASEKPSFIMGGSISQGTP | |
| GTYLTSHNQASYTQETPKPSVGSISLGLPRQQESAKSATLPYIKQEEFSPRSQNSQPEG | |
| LLVRAQHEGVVRGTAGAIQEGSITRGTPTSKISVESIPSLRGSITQGTPALPQTGIPTE | |
| ALVKGSISRMPIEDSSPEKGREEAASKGHVIYEGKSGHILSYDNIKNAREGTRSPRTAH | |
| EISLKRSYESVEGNIKQGMSMRESPVSAPLEGLICRALPRGSPHSDLKERTVLSGSIMQ | |
| GTPRATTESFEDGLKYPKQIKRESPPIRAFEGAITKGKPYDGITTIKEMGRSIHEIPRQ | |
| DILTQESRKTPEVVQSTRPIIEGSISQGTPIKFDNNSGQSAIKHNVKSLITGPSKLSRG | |
| MPPLEIVPENIKVVERGKYEDVKAGETVRSRHTSVVSSGPSVLRSTLHEAPKAQLSPGI | |
| YDDTSARRTPVSYQNTMSRGSPMMNRTSDVTISSNKSTNHERKSTLTPTQRESIPAKSP | |
| VPGVDPVVSHSPFDPHHRGSTAGEVYRSHLPTHLDPAMPFHRALDPAAAAYLFQRQLSP | |
| TPGYPSQYQLYAMENTRQTILNDYITSQQMQVNLRPDVARGLSPREQPLGLPYPATRGI | |
| IDLTNMPPTILVPHPGGTSTPPMDRITYIPGTQITFPPRPYNSASMSPGHPTHLAAAAS | |
| AEREREREREKERERERIAAASSDLYLRPGSEQPGRPGSHGYVRSPSPSVRTQETMLQQ | |
| RPSVFQGTNGTSVITPLDPTAQLRIMPLPAGGPSISQGLPASRYNTAADALAALVDAAA | |
| SAPQMDVSKTKESKHEAARLEENLRSRSAAVSEQQQLEQKTLEVEKRSVQCLYTSSAFP | |
| SGKPQPHSSVVYSEAGKDKGPPPKSRYEEELRTRGKTTITAANFIDVIITRQIASDKDA | |
| RERGSQSSDSSSSLSSHRYETPSDAIEVISPASSPAPPQEKLQTYQPEVVKANQAENDP | |
| TRQYEGPLHHYRPQQESPSPQQQLPPSSQAEGMGQVPRTHRLITLADHICQIITQDFAR | |
| NQVSSQTPQQPPTSTFQNSPSALVSTPVRTKTSNRYSPESQAQSVHHQRPGSRVSPENL | |
| VDKSRGSRPGKSPERSHVSSEPYEPISPPQVPVVHEKQDSLLLLSQRGAEPAEQRNDAR | |
| SPGSISYLPSFFTKLENTSPMVKSKKQEIFRKLNSSGGGDSDMAAAQPGTEIFNLPAVT | |
| TSGSVSSRGHSFADPASNLGLEDIIRKALMGSFDDKVEDHGVVMSQPMGVVPGTANTSV | |
| VTSGETRREEGDPSPHSGGVCKPKLISKSNSRKSKSPIPGQGYLGTERPSSVSSVHSEG | |
| DYHRQTPGWAWEDRPSSTGSTQFPYNPLTMRMLSSTPPTPIACAPSAVNQAAPHQQNRI | |
| WEREPAPLLSAQYETLSDSDD | |

Screening Assays

This disclosure includes methods of screening for compounds that sirtuin activity, particularly compounds that modulate sirtuin activity in cells of the adipocyte lineage, e.g., preadipocytes and adipocytes, e.g., BAT and WAT cells. Useful modulators include agents that increase a sirtuin activity and agents that decrease a sirtuin activity.

Exemplary “sirtuin activities” include deacetylase function (e.g., ability to deacetylate a substrate, e.g., an acetylated histone, or p53), interaction with a sirtuin binding partner, e.g., a transcription factor such as a DNA binding transcription factor, a co-repressor, or a co-activator, interaction with sites on genomic DNA (e.g., by indirect recruitment to promoters) and modulation of transcription (e.g., activation or repression of transcription). Assays for such functions include many known assays and assay described herein. Exemplary assays include: deacetylation assays described in US 20030207325, PCT US2004/001239, and mass spectroscopy methods. Exemplary assays for evaluating interaction with genomic nucleic acid include chromatin immunoprecipitation or “CHiP” assays. Assays for evaluating modulation of transcription include those described in “Gene Expression and Transcript analysis.”

A compound can be evaluated to determine its effect on a biochemical, cellular, or organismal phenotype associated with a metabolic disorder, e.g., obesity, an obesity-related disorder, a fat-related disorder, a disorder characterized by insulin resistance, e.g., Type II diabetes, e.g., as described herein, or other disorder described herein.

One exemplary method includes screening for compounds using a method that includes evaluating the compounds for modulation of a sirtuin activity and evaluating the effect of the compound on a biochemical, cellular, or organismal phenotype associated with a metabolic disorder, e.g., obesity, an obesity-related disorder, or a fat-related disorder, e.g., as described herein. The evaluations can be performed in either order. For example, a library of compounds can be vetted using the first criterion (e.g., modulation of SIRT1 activity) to provide a smaller set of compounds, and then evaluating compounds from the smaller set for an effect on a metabolic phenotype. The vetting can also be done in the opposite order.

Compounds which interact with sirtuins can be identified, e.g., by in vitro or in vivo assays. For example, exemplary in vitro assays for SIRT1 activity include cell free assays, e.g., assays in which an isolated SIRT1 polypeptide (including a polypeptide that includes a fragment of at least 100 amino acids of SIRT1, e.g., a fragment described herein) is contacted with a test compound.

When both the assay for screening a compound for the ability to interact with a sirtuin and the assay for determining effect on sirtuin are performed in vivo, e.g., in cell based assays, the assays can be performed in the same or different cells. For example, one or both of the assays can be performed in tissue culture (e.g., 3T3 cells, 3T3-L1 cells, PC12 cells, or primary fibroblast cultures) or in an organism (e.g., a mammal, e.g., a human).

In preferred embodiments, the assays are performed in the presence of a cofactor of sirtuin such as NAD and/or NAD analogs. In some embodiments, the co-factor is added to the cell culture or in vitro assay, e.g., the NAD and/or an NAD analog to facilitate catalysis. “NAD” refers to nicotinamide adenine dinucleotide. An “NAD analog” as used herein refers to a compound (e.g., a synthetic or naturally occurring chemical, drug, protein, peptide, small organic molecule) which possesses structural similarity to component groups of NAD (e.g., adenine, ribose and phosphate groups) or functional similarity (e.g., supports deacetylating a histone or p53 in the presence of Sir2). For example, an NAD analog can be 3-aminobenzamide or 1,3-dihydroisoquinoline (H. Vaziri et al., EMBO J. 16:6018-6033 (1997)). Another example is iso-nicotinamide.

Described below are exemplary methods for identifying compounds that interact with a sirtuin and can modulate activity or expression of a sirtuin. Compounds can be identified which interact with, e.g., bind to, a sirtuin and increase or decrease at least one sirtuin activity, e.g., deacetylation or interaction with a sirtuin binding partner. For example, deacetylation of a substrate by SIRT1 has been found to decrease adipogenesis.

The phrase “deacetylating a substrate” or “deacetylating a transcription factor” refers to the removal of one or more acetyl groups (e.g., CH₃CO²⁻) from the substrate or transcription factor that is acetylated on at least one amino acid residue. The substrate can be a single amino acid (e.g., an acetylated lysine), a peptide (e.g., a N-terminal peptide of a histone, or an acetylated p53 peptide), or a protein. An acetylated substrate can include a fluorophore, e.g., which can be used to monitor the acetylation states of the substrate. The “Fleur-de-Lys™” substrate from Biomol® includes one such exemplary modification. “Acetylation status” refers to the presence or absence of one or more acetyl groups (e.g., CH₃CO²⁻) at one or more lysine (K) residues of a substrate, e.g., a transcription factor. For example, the presence of an acetyl groups can be found at one or more of: K370, K371, K372, K381, and/or K382 of the p53 sequence. “Altering the acetylation status” refers to adding or removing one or more acetyl groups (e.g., CH₃CO²⁻). For example, adding or removing one or more acetyl groups of p53 at one or more lysine (K) residues, e.g., K370, K371, K372, K381, and/or K382, e.g., from p53 or a fragment thereof that includes one or more of the residues.

A variety of molecules may be utilized to modulate the expression, synthesis, or activity of a mammalian SIR2 homolog or a substrate thereof. Such molecules may include, but are not limited to small organic molecules, peptides, antibodies, nucleic acids, antisense nucleic acids, RNAi, ribozyme molecules, triple helix molecules, and the like.

The following assays provide methods (also referred to herein as “evaluating a compound” or “screening a compound”) for identifying modulators, i.e., candidate or test compounds (e.g., peptides, peptidomimetics, small molecules or other drugs) which interact with and/or modulate activity of a sirtuin, e.g., have a stimulatory or inhibitory effect on, for example, SIRT1 expression and/or activity, or have a stimulatory or inhibitory effect on, for example, the expression or activity of a SIRT1 substrate. Such compounds can be agonists or antagonists of a sirtuin. In preferred embodiments, the screening assays described herein are used to identify candidates which function as SIRT1 agonists. As described herein, such a SIRT1 agonist can be used to reduce adipogenesis. Some of these assays may be performed in animals, e.g., mammals, in organs, in cells. Others may be performed in animals, e.g., mammals, in organs, in cells, in cell extracts, e.g., purified or unpurified nuclear extracts, intracellular extracts, in purified preparations, in cell-free systems, in cell fractions enriched for certain components, e.g., organelles or compounds, or in other systems known in the art.

Some exemplary screening assays for assessing activity or function include one or more of the following features:
use of a transgenic cell, e.g., with a transgene encoding a sirtuin or a mutant thereof;
use of a cell modified to facilitate detection of adipogenesis, e.g., modified to include a reporter of adipogenesis,
use of a mammalian cell that expresses a sirtuin;
use of an enzymatic assay for a sirtuin, e.g., to evaluate deacetylation of a substrate, e.g., an amino acid, a peptide or a protein;
detection of binding to a sirtuin, e.g., by a sirtuin binding partner or a test compound, for example, where the compound is, for example, a peptide, protein, antibody or small organic molecule; e.g., the compound modulates (e.g., stimulates or inhibits) an interaction between sirtuin and a sirtuin-binding partner;
use of proximity assays that detect interaction between a sirtuin and a sirtuin-binding partner, e.g., a protein, e.g., a nuclear protein, e.g., a histone or transcription factor (e.g., p53), or fragments thereof, for example, fluorescence proximity assays;
use of radio-labelled substrates, e.g. ³⁵s, ³H, ¹⁴C, e.g., to determine acetylation status, metabolic status, and so forth; and
use of antibodies specific for certain acetylated or de-acetylated forms of the substrate.

Additional screening assays are described in more detail below.

A “compound” or “test compound” can be any chemical compound, for example, a macromolecule (e.g., a polypeptide, a protein complex, or a nucleic acid) or a small molecule (e.g., an amino acid, a nucleotide, an organic or inorganic compound). The test compound can have a formula weight of less than about 10,000 grams per mole, less than 5,000 grams per mole, less than 1,000 grams per mole, or less than about 500 grams per mole. The test compound can be naturally occurring (e.g., a herb or a nature product), synthetic, or both. Examples of macromolecules are proteins, protein complexes, and glycoproteins, nucleic acids, e.g., DNA, RNA (e.g., double stranded RNA or RNAi) and PNA (peptide nucleic acid). Examples of small molecules are peptides, peptidomimetics (e.g., peptoids), amino acids, amino acid analogs, polynucleotides, polynucleotide analogs, nucleotides, nucleotide analogs, organic or inorganic compounds e.g., heteroorganic or organometallic compounds. One exemplary type of protein compound is an antibody or a modified scaffold domain protein. A test compound can be the only substance assayed by the method described herein. Alternatively, a collection of test compounds can be assayed either consecutively or concurrently by the methods described herein.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like). Additional examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Some exemplary libraries are used to generate variants from a particular lead compound. One method includes generating a combinatorial library in which one or more functional groups of the lead compound are varied, e.g., by derivatization. Thus, the combinatorial library can include a class of compounds which have a common structural feature (e.g., framework).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Test compounds can also be obtained from: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological libraries include libraries of nucleic acids and libraries of proteins. Some nucleic acid libraries encode a diverse set of proteins (e.g., natural and artificial proteins; others provide, for example, functional RNA and DNA molecules such as nucleic acid aptamers or ribozymes. A peptoid library can be made to include structures similar to a peptide library. (See also Lam (1997) Anticancer Drug Des. 12:145). A library of proteins may be produced by an expression library or a display library (e.g., a phage display library).

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310).

In vitro Assays

In some embodiments, interaction with, e.g., binding of a sirtuin can be assayed in vitro. The reaction mixture can include, e.g., a co-factor such as NAD and/or a NAD analog, a substrate or other binding partner or potentially interacting fragment thereof. Exemplary binding partners include PGC1, NcoR, and PPAR-gamma, or interacting fragments thereof. Preferably the binding partner is a direct binding partner.

In other embodiments, the reaction mixture can include a sirtuin-binding partner, e.g., a transcription factor, e.g., a transcription and compounds can be screened, e.g., in an in vitro assay, to evaluate the ability of a test compound to modulate interaction between a sirtuin and a sirtuin-binding partner. This type of assay can be accomplished, for example, by coupling one of the components, with a radioisotope or enzymatic label such that binding of the labeled component to the other can be determined by detecting the labeled compound in a complex. A component can be labeled with ¹²⁵I, ³⁵S, ¹⁴C, or ³H, either directly or indirectly, and the radioisotope detected by direct counting of radioemmission or by scintillation counting. Alternatively, a component can be enzymatically labeled with, for example, horseradish peroxidase, alkaline phosphatase, or luciferase, and the enzymatic label detected by determination of conversion of an appropriate substrate to product. Competition assays can also be used to evaluate a physical interaction between a test compound and a target.

Cell-free assays involve preparing a reaction mixture of the target protein (e.g., SIRT1) and the test compound under conditions and for a time sufficient to allow the two components to interact and bind, thus forming a complex that can be removed and/or detected.

The interaction between two molecules can also be detected, e.g., using a fluorescence assay in which at least one molecule is fluorescently labeled. One example of such an assay includes fluorescence energy transfer (FET or FRET for fluorescence resonance energy transfer) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A FET binding event can be conveniently measured through standard fluorometric detection means well known in the art (e.g., using a fluorimeter).

Another example of a fluorescence assay is fluorescence polarization (FP). For FP, only one component needs to be labeled. A binding interaction is detected by a change in molecular size of the labeled component. The size change alters the tumbling rate of the component in solution and is detected as a change in FP. See, e.g., Nasir et al. (1999) Comb Chem HTS 2:177-190; Jameson et al. (1995) Methods Enzymol 246:283; Seethala et al. (1998) Anal Biochem. 255:257. Fluorescence polarization can be monitored in multiwell plates, e.g., using the Tecan Polarion™ reader. See, e.g., Parker et al. (2000) Journal of Biomolecular Screening 5 :77-88; and Shoeman, et al. (1999) 38, 16802-16809.

In another embodiment, determining the ability of the sirtuin to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, a sirtuin is anchored onto a solid phase. The sirtuin/test compound complexes anchored on the solid phase can be detected at the end of the reaction, e.g., the binding reaction. For example, SIRT1 can be anchored onto a solid surface, and the test compound, (which is not anchored), can be labeled, either directly or indirectly, with detectable labels discussed herein.

It may be desirable to immobilize either the sirtuin or a sirtuin binding partner to facilitate separation of complexed from uncomplexed forms of one or both of the proteins, as well as to accommodate automation of the assay. Binding of a test compound to a sirtuin, or interaction of a sirtuin with a second component in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/mammalian homolog of a SIR2 fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or sirtuin, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of sirtuin binding or activity determined using standard techniques.

Other techniques for immobilizing either a sirtuin or a target molecule on matrices include using conjugation of biotin and streptavidin. Biotinylated sirtuin or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface, e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with a sirtuin or target molecules but which do not interfere with binding of the sirtuin to its target molecule. Such antibodies can be derivatized to the wells of the plate, and unbound target or the sirtuin trapped in the wells by antibody conjugation. Methods for detecting such complexes, in addition to those described above for the GST-immobilized complexes, include immunodetection of complexes using antibodies reactive with the sirtuin or target molecule, as well as enzyme-linked assays which rely on detecting an enzymatic activity associated with the sirtuin or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the sirtuin or biologically active portion thereof with a known compound which binds a sirtuin to form an assay mixture, contacting the assay mixture with a test compound, and determining the ability of the test compound to interact with a sirtuin, wherein determining the ability of the test compound to interact with the sirtuin includes determining the ability of the test compound to preferentially bind to the sirtuin or biologically active portion thereof, or to modulate the activity of a target molecule, as compared to the known compound.

The target molecules can, in vivo, interact with one or more cellular macromolecules, such as proteins. For the purposes of this discussion, such cellular and extracellular macromolecules are referred to herein as “binding partners.” Compounds that disrupt such interactions can be useful in regulating the activity of the target product. Such compounds can include, but are not limited to molecules such as antibodies, peptides, and small molecules. Exemplary targets/products for use in this embodiment include the sirtuin binding partners.

To identify compounds that interfere with the interaction between the target product and its binding partner(s), for example, a reaction mixture containing the target product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target and its cellular or extracellular binding partner. Control reaction mixtures are incubated without the test compound or with a placebo. The formation of any complexes between the target product and the cellular or extracellular binding partner is then detected. The formation of a complex in the control reaction, but not in the reaction mixture containing the test compound, indicates that the compound interferes with the interaction of the target product and the interactive binding partner. Additionally, complex formation within reaction mixtures containing the test compound and normal target product can also be compared to complex formation within reaction mixtures containing the test compound and mutant target product. This comparison can be important in those cases wherein it is desirable to identify compounds that disrupt interactions of mutant but not normal target products.

These assays can be conducted in a heterogeneous or homogeneous format. Heterogeneous assays involve anchoring either the target product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target product or the partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment, a homogeneous assay can be used. For example, a preformed complex of the target product and the interactive cellular or extracellular binding partner product is prepared in that either the target products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target product-binding partner interaction can be identified.

In yet another aspect, the sirtuin can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with SIRT1 (“SIRT1-binding proteins”) and are involved in SIRT1 activity. Such SIRT1 binding partners can be activators or inhibitors of signals or transcriptional control.

The two-hybrid system is based on the modular nature of most transcription factors, which consist of separable DNA-binding and activation domains. Briefly, the assay utilizes two different DNA constructs. In one construct, the gene that codes for a SIRT1 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the sirtuin can be the fused to the activator domain.) If the “bait” and the “prey” proteins are able to interact, in vivo, forming a sirtuin-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein Which interacts with the sirtuin. In another embodiment, the two-hybrid assay is used to monitor an interaction between two components, e.g., a sirtuin and, e.g., p53, that are known to interact. The two hybrid assay is conducted in the presence of a test compound, and the assay is used to determine whether the test compound enhances or diminishes the interaction between the components.

In another embodiment, modulators of sirtuin gene expression are identified. For example, a cell or cell free mixture is contacted with a candidate compound and the expression of the mammalian homolog of a SIR2 mRNA or protein evaluated relative to the level of expression of sirtuin mRNA or protein in the absence of the candidate compound. When expression of the mammalian homolog of a SIR2 mRNA or protein is greater in the presence of the candidate compound than in its absence, the candidate compound is identified as a stimulator of a mammalian homolog of a SIR2 mRNA or protein expression. Alternatively, when expression of the mammalian homolog of a SIR2 mRNA or protein is less (statistically significantly less) in the presence of the candidate compound than in its absence, the candidate compound is identified as an inhibitor of the SIRT1 mRNA or protein expression. The level of the mammalian homolog of a SIR2 mRNA or protein expression can be determined by methods for detecting mammalian homolog of a SIR2 mRNA or protein, e.g., using probes or antibodies, e.g., labelled probes or antibodies.

Cell-Based Assays

In another embodiment, the assay, e.g., the assay for selecting compounds which interact with sirtuins can be a cell-based assay. Useful assays include assays in which a marker of adipocyte differentiation, a fat or lipid parameter is measured. The cell based assay can include contacting a cell expressing a sirtuin with a test compound and determining the ability of the test compound to modulate (e.g. stimulate or inhibit) an activity of a sirtuin, and/or determine the ability of the test compound to modulate expression of a sirtuin, e.g., by detecting sirtuin nucleic acids (e.g., mRNA or cDNA) or proteins in the cell. Determining the ability of the test compound to modulate sirtuin activity can be accomplished, for example, by determining the ability of the sirtuin to bind to or interact with the test molecule, and by determining the ability of the test molecule to modulate adipogenesis. Cell-based systems can be used to identify compounds that decrease expression and/or activity and/or effect of a sirtuin. Such cells can be recombinant or non-recombinant, such as cell lines that express the sirtuin gene. In some embodiments, the cells can be recombinant or non-recombinant cells which express a sirtuin binding partner. Exemplary systems include mammalian or yeast cells that express a sirtuin, e.g., from a recombinant nucleic acid. In utilizing such systems, cells are exposed to compounds suspected of increasing expression and/or activity of a sirtuin. After exposure, the cells are assayed, for example, for sirtuin expression or activity. Alternatively, the cells may also be assayed for the activation or inhibition of the deacetylation function of a sirtuin, or modulation of adipogenesis. In one embodiment, the visual assessment can be used as evidence of adipogenesis.

A cell can be from a stable cell line or a primary culture obtained from an organism, e:g., a organism treated with the test compound.

In addition to cell-based and in vitro assay systems, non-human organisms, e.g., transgenic non-human organisms or a model organism, can also be used. A transgenic organism is one in which a heterologous DNA sequence is chromosomally integrated into the germ cells of the animal. A transgenic organism will also have the transgene integrated into the chromosomes of its somatic cells. Organisms of any species, including, but not limited to: yeast, worms, flies, fish, reptiles, birds, mammals (e.g., mice, rats, rabbits, guinea pigs, pigs, micro-pigs, and goats), and non-human primates (e.g., baboons, monkeys, chimpanzees) may be used in the methods described herein.

A transgenic cell or animal used in the methods of the invention can include a transgene that encodes, e.g., a sirtuin. The transgene can encode a protein that is normally exogenous to the transgenic cell or animal, including a human protein, e.g., a human sirtuin, e.g., SIRT1. The transgene can be linked to a heterologous or a native promoter. Methods of making transgenic cells and animals are known in the art.

Accordingly, in another embodiment, the invention features a method of identifying a compound as a candidate of treatment of a metabolic disorder, e.g., obesity, an obesity-related disorder, or a fat-related disorder. The method includes: providing a compound which interacts with, e.g., binds to, a sirtuin; evaluating the effect of the compound on adipocyte differentiation; and further evaluating the effect of the test compound on a subject, e.g., an animal model, e.g., an animal model for a metabolic disorder, e.g., obesity. The interaction between a test compound and the sirtuin can be evaluated by any of the methods described herein, e.g., using cell-based assays or cell-free in vitro assays.

Promoters and Reporters

Reporter genes can be made by operably linking a regulatory sequence to a sequence encoding a reporter gene. A number of methods are available for designing reporter genes. For example, the sequence encoding the reporter protein can be linked in frame to all or part of the sequence that is normally regulated by the regulatory sequence. Such constructs can be referred to as translational fusions. It is also possible to link the sequence encoding the reporter protein to only regulatory sequences, e.g., the 5′ untranslated region, TATA box, and/or sequences upstream of the mRNA start site. Such constructs can be referred to as transcriptional fusions. Still other reporter genes can be constructed by inserting one or more copies (e.g., a multimer of three, four, or six copies) of a regulatory sequence into a neutral or characterized promoter.

Synthetic promoters can include one or more multimerized sites, e.g., a site that is specifically recognized by a transcription factor described herein, e.g., a PPAR (e.g., PPAR-alpha, PPAR-gamma, or PPAR-delta) or a C/EBP (e.g., CEPB. For example, an exemplary PPRE (PPAR-gamma response element) includes TTGCCCTTG, another includes TCACCCTTG.

Reporter constructs can be used to evaluate expression of any gene described herein or any gene whose expression is correlated with adipocyte or keratinocyte function.

The UCP1 promoter can include about nucleotides 142052800 to 142058843 on the plus strand of human chromosome 4. Useful promoter nucleic acids can include at least about 500, 800, 1 kb, 2 kb, 3 kb, or 4 kb of the above region, for example, regions of such size that include the mRNA start site, the TATA box, or the UCP1 ATG. For example, the promoter can terminate at the mRNA start site, the TATA box, or the UCP1 ATG.

The leptin promoter can include about nucleotides 127427395 to 127435395 on the plus strand of human chromosome 7. Useful promoter nucleic acids can include at least about 500, 800, 1 kb, 2 kb, 3 kb, 4 kb, 6 kb, or 8 kb of the above region, for example, regions of such size that include the mRNA start site, the TATA box, or the leptin ATG. For example, the promoter can terminate at the mRNA start site, the TATA box, or the leptin ATG.

The PPARG promoter can include about nucleotides 12,298,254 to 12,306,254 on the plus strand of human chromosome 3. Useful promoter nucleic acids can include at least about 500, 800, 1 kb, 2 kb, 3 kb, 4 kb, 6 kb, or 8 kb of the above region, for example, regions of such size that include the mRNA start site, the TATA box, or the PPARG ATG. For example, the promoter can terminate at the mRNA start site, the TATA box, or the PPARG ATG.

The resistin (RETN) promoter can include about nucleotides 7,631,989 to 7,639,989 on the plus strand of human chromosome 19. Useful promoter nucleic acids can include at least about 500, 800, 1 kb, 2 kb, 3 kb, 4 kb, 6 kb, or 8 kb of the above region, for example, regions of such size that include the mRNA start site, the TATA box, or the RETN ATG. For example, the promoter can terminate at the mRNA start site, the TATA box, or the RETN ATG.

Still other genes of interest include: genes that encode C/EBP-α, C/EBP-δ and Ap2.

Exemplary reporter proteins include chloramphenicol acetyltransferase, green fluorescent protein and other fluorescent proteins (e.g., artificial variants of GFP), beta-lactamase, beta-galactosidase, luciferase, and so forth. The reporter protein can be any protein other than the protein encoded by the endogenous gene that is subject to analysis. Epitope tags can also be used.

Pharmaceutical Compositions

An agent that modulates activity of SIRT1 or other sirtuin can be incorporated into a pharmaceutical composition, e.g., a composition that includes a pharmaceutically acceptable carrier.

As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Examples of modulators of sirtuin activity include nucleic acids that encode a sirtuin, or fragments thereof, nucleic acids that inhibit sirtuin gene expression, and polypeptides that have a sirtuin activity, fragments thereof, as well as antibodies that bind to and/or inhibit a sirtuin (e.g., SIRT1). Such modulators can be provided as a pharmaceutical composition. Other types of modulators include small molecule inhibitors and activators, e.g., as described herein.

A therapeutically effective amount of protein or polypeptide (i.e., an effective dosage) includes ranges, e.g., from about 0.001 to 30 mg/kg body weight, preferably about 0.01 to 25 mg/kg body weight. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

In one embodiment, a composition that includes a modulator of SIRT1 activity is used to modulate (e.g., increase) the amount of subcutaneous fat in a tissue, e.g., in facial tissue or in other surface-associated tissue of the neck, hand, leg, or lips. The modulator (e.g., SIRT1 inhibitor) can be used to increase the rigidity, water retention, or support properties of the tissue. For example, the composition can be applied topically, e.g., in association with another agent, e.g., for surface-associated tissue treatment. The composition may also be injected subcutaneously, e.g., within the region where an alteration in subcutaneous fat is desired.

Compositions can also be used to modulate fertility.

An additional treatment includes using a modulator of SIRT1 activity to alter keratinocyte aging or appearance, e.g., by administering (e.g., topically applying the modulator to a surface site, e.g., in an amount effective to modulate keratinocyte aging.

Exemplary Activators of SIRT1 Activity

One exemplary class of SIRT1 activators include polyphenols, e.g., a flavone, stilbene, flavanone, cetchin, chalcone, isoflavone, anthocyanidin, or tannin. In some instances, the SIRT1 activator is a compound of formula (I): 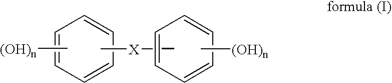
wherein;
X is, for example, alkenyl, C(O)CH═CH, or a hydroxy pyranone fused to one of the phenyl moieties to form a flavone; and
each n is, e.g., independently 1-3.

For example, the compound can be a polyhydroxy stillbene (e.g., polyhydroxy-trans-stillbene) as shown in formula (II), a polyhydroxy chalcone as shown in formula (III), or a polyhydroxyflavone as shown in formula (IV). In general, the compound is substituted with at least 2, preferably 3, 4, of 5 hydroxy moieties.

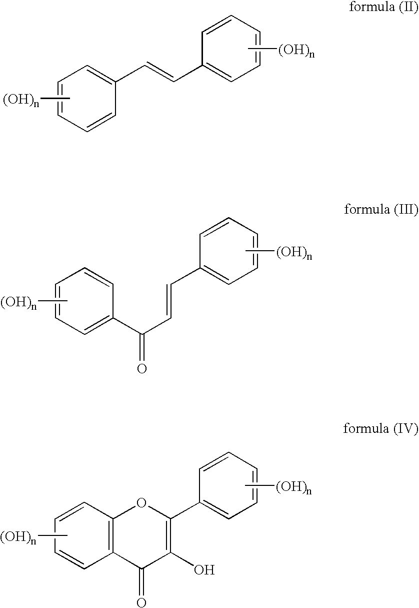

Exemplary compounds include resveratrol (3,5,4′-trihydroxy-tans-stilbene), butein (3,4,2′,4′-tetrahydroxychalcone); piceatannol (3,5,3′, 4′-tetrahydroxy-trans-stilbene); isoliquiritigenin (4,2′,4′-trihydroxychalcone); fisetin (3,7,3′,4′-tetrahydroxyflavone); and quercetin (3,5,7,3′,4′-pentahydroxyflavone). See, e.g., Howitz (2003) Nature 425:191-196.

In one embodiment, such compounds are provided in a non-liquid form, e.g., a semi-solid form, e.g., a tablet or gel. In another embodiment, the compounds is in liquid form, e.g., a beverage, e.g., a non-alcoholic beverage, e.g., a beverage that does or does not include a natural by product of grapes. The compounds can be made synthetically or by extraction from a natural product.

The compound can be a compound which increases SIRT1 activity, e.g., at least 0.5, 1, 2, 3, 4, 8, 10, or 12-fold, e.g., between 2 and 15-fold.

In certain implementations, compounds of this class, e.g., a trans-stilbene such as resveratrol, are administered in a dosage of at least 0.5, 1, 5, 10, 20, 50, or 100 mg per day to a subject, e.g., a human subject. The dosage can be provided in one or more boluses.

Exemplary Inhibitors of SIRT1 Activity

Exemplary inhibitors of SIRT1 activity include Compound A3 (8,9-dihydroxy-6H-(1)benzofuro[3,2-c]chromen-6-one), Compounds M15 (1-[(4-methoxy-2-nitro-phenylimino)-methyl]-naphthalene-2-ol) and Sirtinol (2-[(2-hydroxy-naphthalen-1-ylmethylene)-amino]-N-(1-phenyl-ethyl)-benzamide). Such compounds are available, e.g., from ChemBridge or can be synthesized. See, e.g., Grozinger et al. (2001) J. Biol. Chem., Vol. 276, Issue 42, 38837-3884.

Additional exemplary compounds are described in WO 03/046207. Some exemplary compounds have the structure of Formula V:

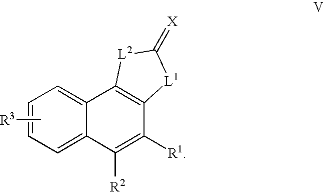

In Formula V, the letter X is a member selected from the group consisting of O and S. The symbols L¹ and L² each represent members independently selected from the group consisting of O, S, ethylene and propylene, substituted with 0-2 R groups, wherein exactly one of the symbols L¹ and L² represents a member selected from the group consisting of O and S. Each instance of the letter R of symbols L¹ and L² independently represents a member selected from the group consisting of C₁₋₆alkyl, C₂₋₆alkenyl and —CO₂R⁴. The symbols R¹ and R² each represent members independently selected from the group consisting of hydrogen, C₁₋₆alkoxy, C₁₋₆alkoxy-aryl and hydroxy. Alternatively, the symbols R¹ and R² are taken together with the carbons to which they are attached to form a six-membered lactone ring.

The symbol R³ represents a member selected from the group consisting of hydrogen, C₁₋₆alkyl, aryl, —OR⁴, —NR⁴R⁴, —CO₂R⁴, —C(O)R⁴, —C(O)NR⁴R⁴, —CN, —NO₂ and halogen. Each instance of the symbol R⁴ independently represents a member selected from the group consisting of hydrogen and C₁₋₆alkyl.

The compound of Formula V can have the following structure:

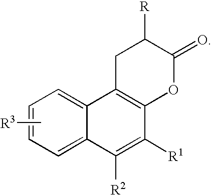

In this case, the symbol R¹ is a member selected from the group consisting of hydrogen, C₁₋₆alkoxy and C₀₋₆alkoxy-aryl; the symbol R² is a member selected from the group consisting of hydrogen and hydroxy; the symbol R³ is a member selected from the group consisting of hydrogen and —OR⁴; and the symbol R⁴ is C₁₋₆alkyl.

In another variation, the symbol R¹ is a member selected from the group consisting of C₁₋₆alkoxy, C₀₋₆alkoxy-aryl and hydroxy. For example, the symbol R¹ is a member selected from the group consisting of hydroxy, methoxy and benzyloxy. In another preferred embodiment, the term aryl is a member selected from the group consisting of phenyl and naphthyl.

Another exemplary compound has the structure of Formula VI:

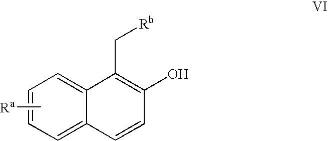

In Formula VI, the symbol R^{a} is a member selected from the group consisting of hydrogen, C₁₋₆alkyl, aryl, —OR^{e}, —NR^{e}R^{e}, —CO₂R^{e}, —C(O)R^{e}, —C(O)NR^{e}R^{e}, —CN, —NO₂ and halogen, while the symbol R^{b} is a member selected from the group consisting of:

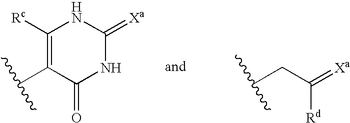

In the components above, the symbol X^{a} can be O, S, or NR^{e}. The symbol R^{c} can be hydrogen, C₁₋₆alkyl and aryl optionally substituted with a hydrogen, C₁₋₆alkyl, aryl, —Ore, —NR^{e}R^{e}, —CN, —NO₂ or halogen. The symbol R^{d} can be hydrogen, C₁₋₆alkyl, aryl, —Ore, —NR^{e}R^{e}, or halogen. Each instance of the symbol R^{e} can be independently hydrogen or C₁₋₆alkyl. In one embodiment, a compound of Formula VI has the following structure 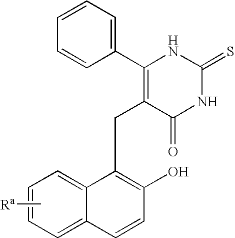
Sirtuin Modulating Nucleic Acids

A sirtuin modulator can be a siRNA, anti-sense RNA, or a ribozyme, which can decreases the expression of the sirtuin. In some aspects, a cell or subject can be treated with a compound that modulates the expression of a gene, e.g., a nucleic acid which modulates, e.g., decreases, expression of a polypeptide which inhibits a sirtuin. Such approaches include oligonucleotide-based therapies such as RNA interference, antisense, ribozymes, and triple helices.

Gene expression can be modified by gene silencing using double-strand RNA (Sharp (1999) Genes and Development 13: 139-141). RNAi, otherwise known as double-stranded RNA interference (dsRNAi) or small interfering RNA (siRNA), has been extensively documented in a number of organisms, including mammalian cells, the nematode C. elegans (Fire, A., et al, Nature, 391, 806-811, 1998).

dsRNA can be delivered to cells or to an organism to antagonize a sirtuin or other protein described herein. For example, a dsRNA that is complementary to a SIRT1 nucleic acid can silence protein expression of SIRT1. The dsRNA can include a region that is complementary to a coding region of a SIRT1 nucleic acid, e.g., a 5′ coding region, a region encoding a sirtuin core domain, a 3′ coding region, or a non-coding region, e.g., a 5′ or 3′ untranslated region. dsRNA can be produced, e.g., by transcribing a cassette (in vitro or in vivo) in both directions, for example, by including a T7 promoter on either side of the cassette. The insert in the cassette is selected so that it includes a sequence complementary to the SIRT1 nucleic acid. The sequence need not be full length, for example, an exon, or between 19-50 nucleotides or 50-200 nucleotides. The sequence can be from the 5′ half of the transcript, e.g., within 1000, 600, 400, or 300 nucleotides of the ATG. See also, the HISCRIBE™ RNAi Transcription Kit (New England Biolabs, MA) and Fire, A. (1999) Trends Genet. 15, 358-363. dsRNA can be digested into smaller fragments. See, e.g., U.S. patent application 2002-0086356 and 2003-0084471.

In one embodiment, an siRNA is used. siRNAs are small double stranded RNAs (dsRNAs) that optionally include overhangs. For example, the duplex region is about 18 to 25 nucleotides in length, e.g., about 19, 20, 21, 22, 23, or 24 nucleotides in length. Typically, the siRNA sequences are exactly complementary to the target mRNA. It may also be possible to agonize activity of a sirtuin by using an siRNA to inhibit a negative regulator of the sirtuin.

Double-stranded inhibitory RNA can also be used to selectively reduce the expression of one allele of a gene and not the other, thereby achieving an approximate 50% reduction in the expression of a sirtuin antagonist polypeptide. See Garrus et al. (2001), Cell 107(l):55-65.

“Ribozymes” are enzymatic RNA molecules which cleave at specific sites in RNA. Ribozymes that can specifically cleave nucleic acids that encode or that are required for the expression of sirtuins may be designed according to well-known methods.

Antibodies

Immunoglobulins can also be produced that bind to a sirtuin or a sirtuin binding partner (e.g., a transcription factor that interacts with a sirtuin). For example, an immunoglobulin can bind to a sirtuin and prevent sirtuin enzymatic activity or an interaction between a sirtuin and a sirtuin binding partner (e.g., NCoR or PGC1). In a preferred embodiment, the immunoglobulin is human, humanized, deimmunized, or otherwise non-antigenic in the subject.

An immunoglobulin can be, for example, an antibody or an antigen-binding fragment thereof. As used herein, the term “immunoglobulin” refers to a protein consisting of one or more polypeptides that include one or more immunoglobulin variable domain sequences. A typical immunoglobulin includes at least a heavy chain immunoglobulin variable domain and a light chain immunoglobulin variable domain. An immunoglobulin protein can be encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 KDa or 214 amino acids) are encoded by a variable region gene at the NH2-terminus (about 110 amino acids) and a kappa or lambda constant region gene at the COOH-terminus. Full-length immunoglobulin “heavy chains” (about 50 KDa or 446 amino acids), are similarly encoded by a variable region gene (about 116 amino acids) and one of the other aforementioned constant region genes, e.g., gamma (encoding about 330 amino acids). The term “antigen-binding fragment” of an antibody (or simply “antibody portion,” or “fragment”), as used herein, refers to one or more fragments of a full-length antibody that retain the ability to specifically bind to the antigen. Examples of antigen-binding fragments include: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also encompassed within the term “antigen-binding fragment” of an antibody. These antibody fragments are obtained using conventional techniques known to those with skill in the art, and the fragments are screened for utility in the same manner as are intact antibodies.

In one embodiment, the antibody against a sirtuin or other protein is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey). Preferably, the non-human antibody is a rodent (mouse or rat antibody). Method of producing rodent antibodies are known in the art. Non-human antibodies can be modified, e.g., humanized or deimmunized. Human monoclonal antibodies can be generated using transgenic mice carrying the human immunoglobulin genes rather than the mouse system (see, e.g., WO 91/00906 and WO 92/03918). Other methods for generating immunoglobulin ligands include phage display (e.g., as described in U.S. Pat. No. 5,223,409 and WO 92/20791).

Artificial Transcription Factors

Artificial transcription factors can also be used to regulate genes that are regulated by SIRT1, NCoR, PGC1, or PPAR-gamma. For example, an artificial transcription factor that has the binding specificity of PPAR-gamma can be used to substitute for or augment PPAR-gamma function. For example, the artificial transcription factor (e.g., that includes one or more zinc finger domains) can be engineered to bind to a nucleic acid sequence recognized by PPAR-gamma, e.g., TTGCCCTTG or TCACCCTTG.

The protein can be designed or selected from a library. The protein can include one or more zinc finger domains. For example, the protein can be prepared by selection in vitro (e.g., using phage display, U.S. Pat. No. 6,534,261) or in vivo, or by design based on a recognition code (see, e.g., WO 00/42219 and U.S. Pat. No. 6,511,808). See, e.g., Rebar et al. (1996) Methods Enzymol 267:129; Greisman and Pabo (1997) Science 275:657; Isalan et al. (2001) Nat. Biotechnol 19:656; and Wu et al. (1995) Proc. Nat. Acad. Sci. USA 92:344 for, among other things, methods for creating libraries of varied zinc finger domains.

Optionally, the zinc finger protein can be fused to a transcriptional regulatory domain, e.g., an activation domain to activate transcription or a repression domain to repress transcription. The zinc finger protein can itself be encoded by a heterologous nucleic acid that is delivered to a cell or the protein itself can be delivered to a cell (see, e.g., U.S. Pat. No. 6,534,261. The heterologous nucleic acid that includes a sequence encoding the zinc finger protein can be operably linked to an inducible promoter, e.g., to enable fine control of the level of the zinc finger protein in the cell.

Zinc finger proteins or other artificial transcription factors can also be engineered to recruit SIRT1 function. For example, the proteins can by physically associated with a SIRT1-interacting fragment of NCoR, e.g., by a translational fusion, or can be physically associated with a NCoR-interacting fragment of SIRT1. The proteins may bind with an affinity of less than 5 nM, e.g., less than 1 or 0.1 nM. Such fragments of SIRT1 and NCoR are described herein.

Gene and Cell-Based Therapeutics

Nucleic acid molecules (e.g., DNA molecules) that nucleic acid agents for modulating sirtuin function can be inserted into a variety of DNA constructs and vectors for the purposes of gene therapy. Vectors include plasmids, cosmids, artificial chromosomes, viral elements, and RNA vectors (e.g., based on RNA virus genomes). The vector can be competent to replicate in a host cell or to integrate into a host DNA. Viral vectors include, e.g., replication defective retroviruses, adenoviruses and adeno-associated viruses.

Examples of vectors include replication defective retroviral vectors, adenoviral vectors and adeno-associated viral vectors. Adenoviral vectors suitable for use by the methods of the invention include (Ad.RSV.lacZ), which includes the Rous sarcoma virus promoter and the lacZ reporter gene as well as (Ad.CMV.lacZ), which includes the cytomegalovirus promoter and the lacZ reporter gene. Methods for the preparation and use of viral vectors are described in WO 96/13597, WO 96/33281, WO 97/15679, and Trapnell et al., Curr. Opin. Biotechnol. 5(6):617-625, 1994, the contents of which are incorporated herein by reference.

A gene therapy vector is a vector designed for administration to a subject, e.g., a mammal, such that a cell of the subject is able to express a therapeutic gene contained in the vector. The therapeutic gene may encode a protein (e.g. SIRT1). The therapeutic gene can also be used to provide a non-coding transcript, e.g., an antisense RNA (e.g., an RNA anti-sense to a sirtuin gene, e.g., a SIRT1 gene, a ribozyme, or a dsRNA.

The gene therapy vector can contain regulatory elements, e.g., a 5′ regulatory element, an enhancer, a promoter, a 5′ untranslated region, a signal sequence, a 3′ untranslated region, a polyadenylation site, and a 3′ regulatory region. For example, the 5′ regulatory element, enhancer or promoter can regulate transcription of the DNA encoding the therapeutic polypeptide or other transcript. The regulation can be tissue specific. For example, the regulation can restrict transcription of the desired gene to brain cells, e.g., cortical neurons or glial cells; hematopoietic cells; or endothelial cells. Alternatively, regulatory elements can be included that respond to an exogenous drug, e.g., a steroid, tetracycline, or the like. Thus, the level and timing of expression of the therapeutic nucleic acid can be controlled.

Gene therapy vectors can be prepared for delivery as naked nucleic acid, as a component of a virus, or of an inactivated virus, or as the contents of a liposome or other delivery vehicle. See, e.g., U.S. 2003-0143266 and 2002-0150626. In one embodiment, the nucleic acid is formulated in a lipid-protein-sugar matrix to form microparticles., e.g., having a diameter between 50 nm to 10 micrometers. The particles may be prepared using any known lipid (e.g., dipalmitoylphosphatidylcholine, DPPC), protein (e.g., albumin), or sugar (e.g., lactose).

The gene therapy vectors can be delivered using a viral system. Exemplary viral vectors include vectors from retroviruses, e.g., Moloney retrovirus, adenoviruses, adeno-associated viruses, and lentiviruses, e.g., Herpes simplex viruses (HSV). HSV, for example, is potentially useful for infecting nervous system cells. See, e.g., U.S. 2003-0147854, 2002-0090716, 2003-0039636, 2002-0068362, and 2003-0104626. The gene delivery agent, e.g., a viral vector, can be produced from recombinant cells which produce the gene delivery system.

A gene therapy vector can be administered to a subject, for example, by intravenous injection, by local administration (see U.S. Pat. No. 5,328,470) or by stereotactic injection (see e.g., Chen et al. (1994) Proc. Natl. Acad. Sci. USA 91:3054-3057). The gene therapy agent can be further formulated, for example, to delay or prolong the release of the agent by means of a slow release matrix. One method of providing a therapeutic agent, is by inserting a gene therapy vector into cells harvested from a subject. The cells are infected, for example, with a retroviral gene therapy vector, and grown in culture. The subject is then replenished with the infected culture cells. The subject is monitored for recovery and for production of the therapeutic polypeptide or nucleic acid.

Cell based-therapeutic methods include introducing a nucleic acid that provides a therapeutic activity operably linked to a promoter into a cell in culture. The therapeutic nucleic acid can provide the desired modulation of SIRT1 activity in a cultured cell, e.g., an increase or decrease in SIRT1 activity to a cell of the adipocyte lineage. Further, it is also possible to modify cells, e.g., stem cells, using nucleic acid recombination, e.g., to insert a transgene, e.g., a transgene that provides a therapeutic activity. The modified stem cell can be administered to a subject. Methods for cultivating stem cells in vitro are described, e.g., in U.S. application 2002-0081724. In some examples, the stem cells can be induced to differentiate in the subject and express the transgene. For example, the stem cells can be differentiated into liver, adipose, or skeletal muscle cells. The stem cells can be derived from a lineage that produces cells of the desired tissue type, e.g., liver, adipose, or skeletal muscle cells.

As used herein, “antisense therapy” refers to administration or in situ generation of oligonucleotide molecules or their derivatives which specifically hybridize (e.g., bind) under cellular conditions with the cellular mRNA and/or genomic DNA, thereby inhibiting transcription and/or translation of that gene. In general, antisense therapy refers to the range of techniques generally employed in the art, and includes any therapy which relies on specific binding to oligonucleotide sequences. An antisense construct can be delivered, for example, as an expression plasmid which, when transcribed in the cell, produces RNA which is complementary to at least a unique portion of the cellular mRNA.

Antisense RNA, DNA, and ribozyme molecules may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Modifications to nucleic acid molecules may be introduced as a means of increasing intracellular stability and half-life. Exemplary modifications include the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

Gene Expression and Transcript Analysis

Different aspects of the invention can include evaluating expression of one or more genes described herein (e.g., genes encoding aP2, PPAR-gamma, SIRT1, leptin, adiponectin, resistin, C/EPB and UCP1). Expression of a gene can be evaluated by detecting an mRNA, e.g., the transcript from the gene of interest or detecting a protein, e.g., the protein encoded by the gene of interest.

Exemplary methods for evaluating mRNAs include Northern analysis, RT-PCR, microarray hybridization, SAGE, differential display, and monitoring reporter genes. Exemplary methods for evaluating proteins include immunoassays (e.g., ELISAs, immunoprecipitations, Westerns), 2D-gel electrophoresis, and mass spectroscopy. It is possible to evaluate fewer than 100, e.g., less than 20, 10, 5, 4 or 3 different molecular species, e.g., to only evaluate the expression of the gene of interest, although it is typically useful to include at least one or two controls (e.g., a house keeping gene). It is also possible to evaluate multiple molecular species, e.g., in parallel, e.g., at least 10, 50, 20, 100, or more different species. See, e.g., the usage of microarrays, e.g. as described below.

One method for comparing transcripts uses nucleic acid microarrays that include a plurality of addresses, each address having a probe specific for a particular transcript. aAt least one of which is specific for a gene of interest, e.g., a gene encoding aP2, PPAR-gamma, SIRT1, leptin, adiponectin, resistin, C/EPB and UCP1). Such arrays can include at least 100, or 1000, or 5000 different probes, so that a substantial fraction, e.g., at least 10, 25, 50, or 75% of the genes in an organism are evaluated. MRNA can be isolated from a cell or other sample of the organism. The mRNA can be reversed transcribed into labeled cDNA. The labeled cDNAs are hybridized to the nucleic acid microarrays. The arrays are detected to quantitate the amount of cDNA that hybridizes to each probe, thus providing information about the level of each transcript.

Methods for making and using nucleic acid microarrays are well known. For example, nucleic acid arrays can be fabricated by a variety of methods, e.g., photolithographic methods (see, e.g., U.S. Pat. Nos. 5,143,854; 5,510,270; and 5,527,681), mechanical methods (e.g., directed-flow methods as described in U.S. Pat. No. 5,384,261), pin based methods (e.g., as described in U.S. Pat. No. 5,288,514), and bead based techniques (e.g., as described in PCT US/93/04145). The capture probe can be a single-stranded nucleic acid, a double-stranded nucleic acid (e.g., which is denatured prior to or during hybridization), or a nucleic acid having a single-stranded region and a double-stranded region. Preferably, the capture probe is single-stranded. The capture probe can be selected by a variety of criteria, and preferably is designed by a computer program with optimization parameters. The capture probe can be selected to hybridize to a sequence rich (e.g., non-homopolymeric) region of the nucleic acid. The Tₘ of the capture probe can be optimized by prudent selection of the complementarity region and length. Ideally, the Tₘ of all capture probes on the array is similar, e.g., within 20, 10, 5, 3, or 2° C. of one another. A database scan of available sequence information for a species can be used to determine potential cross-hybridization and specificity problems.

The isolated mRNA from samples for comparison can be reversed transcribed and optionally amplified, e.g., by rtPCR, e.g., as described in (U.S. Pat. No. 4,683,202). The nucleic acid can be labeled during amplification, e.g., by the incorporation of a labeled nucleotide. Examples of preferred labels include fluorescent labels, e.g., red-fluorescent dye Cy5 (Amersham) or green-fluorescent dye Cy3 (Amersham), and chemiluminescent labels, e.g., as described in U.S. Pat. No. 4,277,437. Alternatively, the nucleic acid can be labeled with biotin, and detected after hybridization with labeled streptavidin, e.g., streptavidin-phycoerythrin (Molecular Probes).

The labeled nucleic acid can be contacted to the array. In addition, a control nucleic acid or a reference nucleic acid can be contacted to the same array. The control nucleic acid or reference nucleic acid can be labeled with a label other than the sample nucleic acid, e.g., one with a different emission maximum. Labeled nucleic acids can be contacted to an array under hybridization conditions. The array can be washed, and then imaged to detect fluorescence at each address of the array.

A general scheme for producing and evaluating profiles can include the following. The extent of hybridization at an address is represented by a numerical value and stored, e.g., in a vector, a one-dimensional matrix, or one-dimensional array. The vector x has a value for each address of the array. For example, a numerical value for the extent of hybridization at a first address is stored in variable xₐ. The numerical value can be adjusted, e.g., for local background levels, sample amount, and other variations. Nucleic acid is also prepared from a reference sample and hybridized to an array (e.g., the same or a different array), e.g., with multiple addresses. The vector y is construct identically to vector x. The sample expression profile and the reference profile can be compared, e.g., using a mathematical equation that is a function of the two vectors. The comparison can be evaluated as a scalar value, e.g., a score representing similarity of the two profiles. Either or both vectors can be transformed by a matrix in order to add weighting values to different nucleic acids detected by the array.

The expression data can be stored in a database, e.g., a relational database such as a SQL database (e.g., Oracle or Sybase database environments). The database can have multiple tables. For example, raw expression data can be stored in one table, wherein each column corresponds to a nucleic acid being assayed, e.g., an address or an array, and each row corresponds to a sample. A separate table can store identifiers and sample information, e.g., the batch number of the array used, date, and other quality control information.

Other methods for quantitating mRNAs include: quantitative RT-PCR. In addition, two nucleic acid populations can be compared at the molecular level, e.g., using subtractive hybridization or differential display to evaluate differences in mRNA expression, e.g., between a cell of interest and a reference cell.

These and other aspects of the invention are described further in the following examples, which are illustrative and in no way limiting.

### EXAMPLE 1

### 3T3-L1 Murine WAT SIRT1 Expression

We found that SIRT1 is expressed in murine subcutaneous inguinal WAT (hips), epididymal WAT (abdominal cavity), and retroperitoneal WAT (abdominal cavity).

Two SVB male mice, aged 19 weeks, (average weight=37.8 g±0.4 g) were maintained in a temperature-controlled (25° C.) facility with a strict 12 h light/dark cycle and were given free access to food and water. The standard rodent chow was obtained from Purina (diet # 5001; Ralston, Purina) and contained 23% protein, 4.5% fat, 6% fiber, 8% ash, and 56% carbohydrate. Tissues from the mice were harvested in a non-fasting condition according to the MIT Animal Care Committee.

Samples of inguinal, epididymal, and retroperitoneal WAT were taken from the mice and subjected to Western Blot analyses using actin as a control to determine if SIRT1 was expressed in each of the three samples. Protein samples were prepared from WAT adipocytes by lysis in RIPA buffer supplemented with protease inhibitors. Twenty micrograms of protein were resolved in 9% SDS-PAGE and transferred to nitrocellulose membranes. SIRT1 protein was probed with mSIRT1 antibody (Upstate Inc.) and detected by ECL (Amersham). An actin antibody (Santa Cruz) was used in the Western Blot as a control. Pronounced SIRT1 expression levels were detected in each of the three WAT samples.

### EXAMPLE 2

### SIRT1 is Induced in Conditions of Lipolysis

Levels of SIRT1 expression in WAT of six SVB male mice, age 19 weeks, (average weight=37.8 g±0.4 g) were determined under the following conditions. Two mice were sacrificed in a control condition after having been fed and kept at room temperature consistent with conditions of Example 1. Two mice were sacrificed after an overnight fast at room temperature to stimulate lipolysis. Two mice were was sacrificed after having been allowed to feed during overnight exposure to an average temperature of 4° C. to stimulate fat release (lipolysis) and thermogenesis. Samples of epididymal WAT were taken from each mouse and subject to Western Blot analyses using actin as a control as in Example 1. SIRT1 protein expression levels were highest in the mice that had stimulated fat release. SIRT1 expression was thereby shown to be positively associated with a decrease in fat content in WAT.

### EXAMPLE 3

### 3T3-L1 SIRT1 Expression Patterns

1. Materials and Methods.

3T3-L1 Mouse Fibroblasts.

The 3T3-L1 mouse fibroblasts used in this example, and in Examples 4-10 hereinafter, were prepared in accordance with standard protocol as follows. 3T3-L1 cells (ATCC CL-173, Rockville, Md.) were grown to confluence (day 0) in medium A (Dulbecco's modified Eagle's Medium with 10% fetal calf serum, 100 units/ml penicillin, and 100 μg/ml streptomycin). Confluent cell were incubated in medium A containing 2 μM insulin, 1 μM dexamethasone, and 0.25 mM isobuthyl methyl xanthine for two days. Thereafter, confluent fibroblasts were treated with insulin for five days and at the end of such treatment were in a condition for functional study. Adipogenesis was evaluated by analysis of the expression of adipocyte-specific markers and by staining of lipids with Oil Red O (Chawla & Lazar, 1994, Proc. Natl. Acad. Sci. USA 91, 1786-1790).

2. SIRT1 Expression Patterns.

Western Blot analyses of 3T3-L1 fibroblast SIRT1 expression using antibodies and actin control as in Example 1 were performed at days 0 through 5 of the differentiation described above and, SIRT1 expression was detected at confluence (day 0; before hormone addition), reached peak expression at day 5, and decreased thereafter. This finding established that SIRT1 protein is expressed endogenously by preadipocytes and that hormonally-induced adipogenesis inhibited SIRT1 protein expression.

### EXAMPLE 4

### Upregulation of SIRT1 Inhibits 3T3-L1 Adipogenesis

1. Materials and Methods.

Plasmids and Vectors

The plasmids pBABE, Jay P. Morgenstern, Hartmut Land, Nucleic Acids Research, Vol. 18:3587, 1990, and pSUPER (OligoEngine; www.oligoengine.com) were used to transform: (1) certain 3T3-L1 fibroblasts described hereinafter in this example, and (2) certain 3T3-L1 fibroblasts described hereinafter in examples 5-10. The plasmid pBABE was used as a control vector and as a component of a vector comprising mouse mSIRT1 cDNA complementary to human SIRT1 mRNA (GenBank Accession No.: AF214646) (SEQ ID NO: 2).

The plasmid pSUPER was used as a control vector and as a component of vectors comprising either mSIRT1 RNAiWT of the sequence

| | |
|---|---|
| 5′-GATGAAGTTGACCTCCTCA-3′, | (SEQ ID NO:24) |

which interferes with SIRT1 expression, or SIRT1 mRNAi-mut of the sequence

| | |
|---|---|
| 5′-GATGAAGTCGACCTCCTCA-3′, | (SEQ ID NO:25) |

which is a mutation of mSIRT1 RNAiWT (SEQ ID NO: 24). SIRT1 mRNAi-mut has no effect on SIRT1 expression. The referenced RNAi sequences are common to human, mouse and rat SIRT1 mRNA.
2. 3T3-L1 Fibroblast Transformation.

3T3-L1 fibroblasts were transformed through standard techniques with either a pBABE control vector (“control fibroblasts”) or a vector comprising pBABE -mSIRT1 cDNA (“mSIRT1 cDNA fibroblasts”). Western Blot analyses of SIRT1 expression by the control and mSIRT1 cDNA fibroblasts were performed using antibodies and actin control as in Example 1.

mSIRT1 cDNA fibroblasts showed levels of SIRT1 expression that were markedly higher than those of the control fibroblasts. Oil Red O staining of the control and mSIRT1 cDNA fibroblasts showed a marked inhibition in differentiation of the mSIRT1 cDNA fibroblasts when compared to the level of differentiation of the control fibroblasts, establishing that upregulation of SIRT1 inhibited adipogenesis.

### EXAMPLE 5

### Downregulation of SIRT1 Enhances 3T3-L1 Adipogenesis

3T3-L1 fibroblasts were transformed with either a pSUPER control vector (“control fibroblasts”), a vector comprising pSUPER-mSIRT1 RNAi WT (SEQ ID NO: 24) (“mSIRT1 RNAi WT fibroblasts”), or pSUPER-mSIRT1 RNAiMUT (SEQ ID NO: 25), (“mSIRT1 RNAiMUT fibroblasts”). Western Blot analyses of SIRT1 expression by the control, mSIRT1 RNAi WT, and mSIRT1 RNAiMUT fibroblasts were performed using antibodies and actin controls as in Example 1.

mSIRT1 RNAiMUT fibroblasts showed levels of SIRT1 expression that were comparable with those of the control fibroblasts, and which were substantially higher than those of the mSIRT1 RNAi WT fibroblasts. Oil Red O staining of the control, mSIRT1 RNAi WT, and mSIRT1 RNAiMUT fibroblasts showed a marked increase in differentiation of the mSIRT1 RNAi WT fibroblasts when compared to the level of differentiation of the control and mSIRT1 RNAiMUT fibroblasts.

These results established that downregulating SIRT1 enhances adipogenesis.

### EXAMPLE 6

### Upregulating SIRT1 Decreases 3T3-L1 Lipolysis Capacity and Downregulating SIRT1 Increases 3T3-L1 Lipolysis Capacity

The amount of fat generated by adipocyte lipolysis should be proportional to the fat content of the adipocyte. In the experiment of this example, four samples of 3T3-L1 fibroblasts were prepared essentially as in Example 3 (except for the change in protocol noted below), and transformed as in Examples 4 and 5 with either pBABE control vector (“pBABE control fibroblasts”), pBABE-mSIRT1 cDNA (“mSIRT1 cDNA fibroblasts”), pSUPER control vector (“pSUPER control fibroblasts”), or pSUPER-mSIRT1 RNAi WT (SEQ ID NO: 24) (“mSIRT1 RNAi WT fibroblasts”).

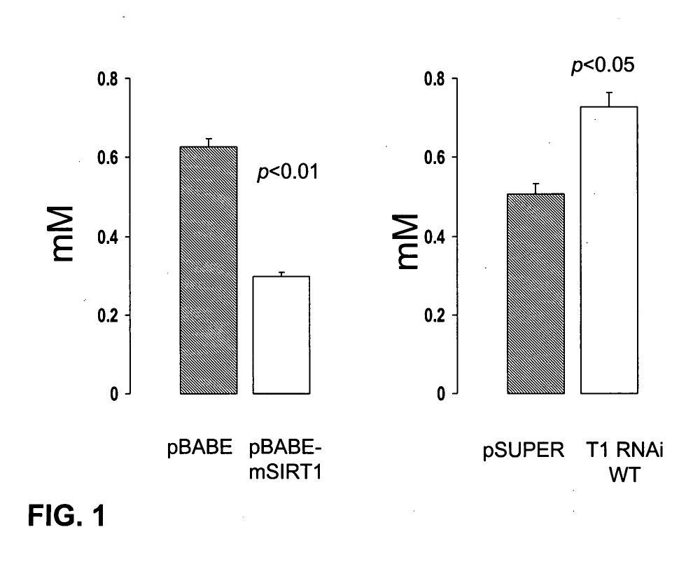

In the experiment of this example, differentiated 3T3-L1 fibroblasts were used at day 7 for study and the fibroblast growth medium was replaced by a Ringers Kreb's buffer which did not contain any fatty acids, but which did contain isoproterenol (¹⁰⁻⁵M) to induce 3T3-L1 fibroblast lipolysis. Fatty acid concentrations in each of the four samples were measured four hours later and the results of these measurements are shown in FIG. 1. Six wells were used for each of the four samples. Results from the different wells were averaged.

Lipolysis levels for the mSIRT1 cDNA fibroblasts were noticeably lower than those of the control (0.297 mM compared to 0.627 mM). Lipolysis in mSIRT1 RNAi WT fibroblasts, in contrast, was higher than in their respective control (0.727 mM compared to 0.508 mM). These observations established that upregulating SIRT1 expression in 3T3-L1 fibroblasts decreased 3T3-L1 lipolysis and that downregulating SIRT1 expression in 3T3-L1 fibroblasts increased 3T3-L1 lipolysis.

### EXAMPLE 7

### Upregulating SIRT1 Inhibits 3T3-L1 Leptin Production

The amount of leptin produced by an adipocyte should be proportional to the fat content of the adipocyte. In the experiment of this example, four samples (prepared in sixplicate) of 3T3-L1 fibroblasts were prepared essentially as in Example 3 (except for the change in protocol noted below), and transformed as in Examples 4 and 5 with either pBABE control vector (“pBABE control fibroblasts”), pBABE-mSIRT1 cDNA (“mSIRT1 cDNA fibroblasts”), pSUPER control vector (“pSUPER control fibroblasts”), or pSUPER-mSIRT1 RNAi WT (SEQ ID NO: 24) (“mSIRT1 RNAi WT fibroblasts”).

Transformed and differentiated 3T3-L1 fibroblasts were used at day 7 for study and the fibroblast growth medium was replaced by a Ringers Kreb's buffer which did not contain any fatty acids. Leptin concentration in each of the four samples was measured twenty-four hours later. Leptin levels for the mSIRT1 cDNA fibroblasts were noticeably lower than those of either the two controls or mSIRT1 RNAi WT fibroblasts. Each sample was compared against its own control. This observation established that upregulating SIRT1 inhibited 3T3-L1 leptin production.

### EXAMPLE 8

### Upregulating SIRT1 Inhibits Synthesis of Adipogenesis Transcription Factors

3T3-L1 fibroblasts were transformed through standard techniques with either a pBABE control vector (“control fibroblasts”) or a vector comprising pBABE-mSIRT1 cDNA (“mSIRT1 cDNA fibroblasts”) in accordance with Example 4. Protein and total RNA was extracted from the differentiated cells. Western Blot analyses of levels of the PPARγ and C/EBPα transcription factors present in the protein extracts from the control and mSIRT1 cDNA fibroblasts were performed using antibodies and actin control as in Example 1. Lower levels of PPARγ were observed in the protein and total RNA extracted from the mSIRT1 cDNA fibroblasts than in the samples extracted from the control fibroblasts.

Semi-quantitative RT-PCR analysis was also conducted to determine levels of the C/EBPβ, C/EBPδ, C/EBPα, PPARγ transcription factors and aP2 (PPARγ target gene) present in the protein and total RNA extracted from the control and mSIRT1 cDNA fibroblasts. In this analysis, GAPDH was used as an internal control for cDNA input in the PCR. Upregulation of SIRT1 decreased levels of all of the assayed transcription factors except C/EBPβ. This finding indicated that SIRT1 may target C/EBPβ and thereby reduce production of the other transcription factors in the C/EBPβ transcriptional cascade.

### EXAMPLE 9

### Downregulating SIRT1 Enhances Levels of Adipogenesis Transcription Factors

3T3-L1 fibroblasts were transformed through standard techniques with either a pSUPER control vector (“control fibroblasts”) or a vector comprising pSUPER-mSIRT1 RNAiWT (SEQ ID NO: 1) (“mSIRT1 RNAiWT fibroblasts”) in accordance with Example 5. Protein and total RNA was extracted from the differentiated cells. Western Blot analyses of levels of the PPARγ and C/EBPα transcription factors present in the protein extracts were performed using antibodies and actin control as in Example 1. Higher levels of PPARγ were observed in the protein and total RNA extracted from the mSIRT1RNAiWT fibroblasts than in the samples extracted from the control fibroblasts.

Semi-quantitative RT-PCR analysis was also conducted to determine levels of the C/EBPβ, C/EBPδ, C/EBPα, PPARγ, and aP2 (PPARγ target gene) present in the protein and total RNA extracted from the control and mSIRT1RNAiWT fibroblasts. In this analysis, GAPDH was used as an internal control for cDNA input in the PCR. Downregulation of SIRT1 was associated with increased levels of all of the assayed transcription factors except C/EBPβ. This finding also indicates that SIRT1 may target C/EBPβ and thereby reduce production of the other transcription factors in the C/EBPβ transcriptional cascade.

### EXAMPLE 10

### SIRT1 Does Not Interfere With C/EBPβ Translocation

SIRT1 may target C/EBPβ to reduce production of the other transcription factors in the C/EBPβ transcriptional cascade by either interfering with C/EBPβ's translocation to the fibroblast nucleus, or by binding to C/EBPβ in the nucleus and thereby reducing C/EBPβ transcriptional activity. To test the first theory, mouse embryonic fibroblasts (MEF's) that were either wild-type (WT) or knock-out (KO) for the SIRT1 gene were differentiated into adipocytes using the techniques described in Example 3. The nuclei of these cells were stained at confluence and prior to hormonal treatment (DAPI, in blue); the cells were also stained for C/EBPβ at the same time. C/EBβ was observed to be diffused in the cell to the same extent in the WT and KO MEF's.

Essentially the same protocol was applied a second time, except that the cells were stained four hours after stimulation with hormonal cocktail, at which point C/EBPβ was present in the nucleus. C/EBP,B was observed to be present to the same extent in the nuclei of the WT and KO MEF's, indicating that SIRT1 does not interfere with C/EBPβ translocation.

### EXAMPLE 11

### SIRT1 Modulates Intracellular Trigylceride Concentration

Fibroblast intracellular trigylceride (“TG”) levels should decrease upon inhibition of fibroblast differentiation resulting from overexpression of SIRT1. Consistent with Examples 4 and 5, it should be possible to measure in a quantitative manner the amount of intracellular triglycerides that can be observed by Oil Red O staining. To do so, 3T3-L1 fibroblasts were prepared as in Example 3 and transformed as in Examples 4 and 5 with either pBABE control vector (“pBABE control fibroblasts”), pBABE-mSIRT1 cDNA (“mSIRT1 cDNA fibroblasts”), pSUPER control vector (“pSUPER control fibroblasts”), or pSUPER-mSIRT1 RNAi WT (SEQ ID NO: 24) (“mSIRT1 RNAi WT fibroblasts”).

At day 7, the cells were lysed in methanol and neutral lipids were extracted by the Folch method and resuspended in isopropanol. Triglyceride levels were measured by calorimetric methods using a commercial kit (Trig/GB #450032, Roche Diagnostics, Indianapolis, Ind.). Six wells per condition were analyzed.

TG levels for the mSIRT1 cDNA fibroblasts were noticeably lower than those of the control. TG levels in mSIRT1 RNAi WT fibroblasts, in contrast, were higher than in their respective control. These observations established that upregulating SIRT1 expression in 3T3-L1 fibroblasts decreased 3T3-L1 TG concentration levels and that downregulating SIRT1 expression in 3T3-L1 fibroblasts increased 3T3-L1 TG concentration levels.

### EXAMPLE 12

### SIRT1 represses C/EBPα-induced leptin transcription in a dose-dependent Manner

The assay described in this example is useful in determining whether a compound that mimics or modulates the activity of a sirtuin affects leptin transcription induced by transcription factors such as the C/EBP transcription factors. For example, the assay is useful in determining whether a compound that mimics or modulates the activity of SIRT1 also represses C/EBPα-induced leptin transcription. The assay also provides a good model to evaluate the activity of a sirtuin (e.g., SIRT1) in vivo.

All cells were transfected with a vector containing 6.5 kb of the leptin promoter cloned upstream of the luciferase gene. Approximately one-half of the cells were co-transfected with either or both of a vector comprising Pv-Sport-C/EBPα or with different doses of a pCMV plasmid containing the full-length human SIRT1 sequence. See, e.g., Hollenberg et al., J Biol Chem. Feb. 21, 1997;272(8):5283-90). The next day, all cells were harvested and luciferase activity was measured in all cells.

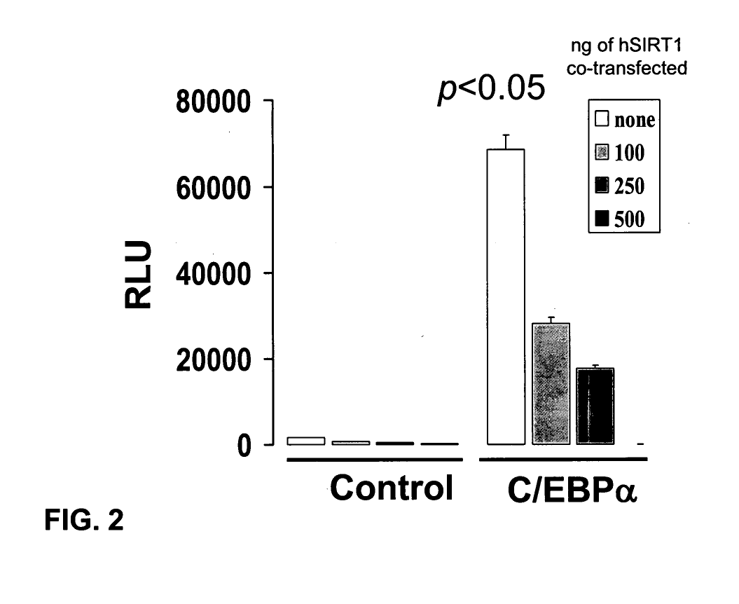

Observed levels of luciferase activity in the transiently transfected cells should have been proportional to the amount of leptin produced by the cell though binding of transcription factors to its promoter. Inhibition of a leptin-related transcription factor as a result of administering an active agent to the transiently transfected cells should have been observed through detection of lower luciferase levels indicative of decreased leptin expression. SIRT1 repressed C/EBPα-induced leptin transcription in a dose-dependent manner. See FIG. 2 or the Table below (Table 1).

TABLE 1

| ng of | | |
|---|---|---|
| hSIRT1 | Control | C/EBPα |
| none | 1600 | 68538 |
| 100 | 722 | 28054 |
| 250 | 352 | 17584 |
| 500 | 253 | 46 |

### EXAMPLE

This disclosure incorporates by reference Picard et al. (2004) Nature doi:10.103/nature02583, Nature. Jun. 17, 2004;429(6993):771-6.

SIRT1 activates a critical component of calorie restriction in mammals; that is, fat mobilization in white adipocytes. Upon food withdrawal Sirt1 protein binds to and represses genes controlled by the fat regulator PPAR-γ (peroxisome proliferator-activated receptor-gamma), including genes mediating fat storage. Sirt1 represses PPAR-γ by docking with its cofactors NCoR (nuclear receptor co-repressor) and SMRT (silencing mediator of retinoid and thyroid hormone receptors). Mobilization of fatty acids from white adipocytes upon fasting is compromised in Sirt1±mice. Repression of PPAR-gamma by Sirt1 is also evident in 3T3-L1 adipocytes, where overexpression of Sirt1 attenuates adipogenesis, and RNA interference of Sirt1 enhances it. In differentiated fat cells, upregulation of Sirt1 triggers lipolysis and loss of fat. As a reduction in fat is sufficient to extend murine lifespan, our results provide a possible molecular pathway connecting calorie restriction to life extension in mammals.

We evaluated whether the mammalian Sir2 orthologue, Sirt1, senses nutrient availability in WAT and mediates corresponding effects on fat accumulation. To probe whether Sirt1 may actually modulate adipogenesis, we used mouse 3T3-L1 fibroblasts as an in vitro model. Adipogenesis in these cells is promoted by the nuclear receptor PPAR-γ. Upon induction of adipogenesis by insulin, dexamethasone and isobutylmethylxanthine, we observed that Sirt1 protein levels increased and peaked at day 5 after hormonal stimulation. Sirt1 expression in 3T3-L1 cells was then modified through retroviral infection with either pBABE-Sirt1 or pSUPER-Sirt1 RNA interference (RNAi) for overexpression (tenfold) or downregulation (sevenfold) of the Sirt1 gene, respectively. 3T3-L1 cells undergo one or two mitotic divisions after induction as a prelude to terminal differentiation. This occurred normally in cells that overexpressed or underexpressed Sirt1 as evaluated by 5-bromodeoxyuridine (BrdU) incorporation. However, compared with cells infected with the control vector, stable 3T3-L1 cells overexpressing Sirt1 accumulated much less fat as determined by Oil red 0 staining after 7 days of differentiation or direct measurement of intracellular triglyceride content. In contrast, downregulation of Sirt1 expression resulted in a significant increase in triglyceride accumulation after differentiation. These results indicate that Sirt1 acts a negative modulator of adipogenesis in the 3T3-L1 model.

We differentiated virally transduced 3T3-L1 cells in the absence of insulin but with rosiglitazone, a very potent, selective PPAR-gamma agonist that acts downstream of the insulin pathway. Although rosiglitazone treatment promoted adipogenesis to a greater extent than the regular differentiation cocktail, it did not alter the phenotypes of cells with increased or decreased levels of Sirt. Accordingly, Sirt1 affects regulators that act downstream of insulin/IGF-1 signalling.

Differentiation of 3T3-L1 fibroblasts increases levels of the transcription factor C/EBP-δ, which stimulates the expression of PPAR-gamma and C/EBP-α. PPAR-gamma induces expression of target genes, such as the fatty-acid-binding protein Ap2 (also known as FABP). Moreover, PPAR-gamma can maintain expression of itself, perhaps by binding to PPAR-gamma sites in the promoter of the PPAR-gamma gene (Pparg). To gain an insight into the mechanisms by which Sirt1 represses fat accretion, we measured protein and messenger RNA expression of key factors in the transcriptional program in the different virus-infected 3T3-L1 adipocytes. We observed a reduction in C/EBP-α, C/EBP-δ and Ap2 mRNA, but not C/EBP-β, upon Sirt1 overexpression. A reduction in PPAR-gamma and C/EBP-alpha was also observed by western blotting. In contrast, cells in which Sirt1 had been downregulated showed higher levels of PPAR-γ, C/EBP-δ, C/EBP-alpha and Ap2. Thus, Sirt1 functions to reduce expression of genes that drive white adipocyte differentiation and fat storage.

We fully differentiated 3T3-L1 cells and subsequently (12 days after induction) applied the Sirt1 activator resveratrol over a wide range of concentrations. After staining the cells for fat content, a strong reduction in fat was observed at 50 and 100 μM resveratrol. The loss of fat was due to activation of Sirt1, because there was no drug-mediated fat reduction in cells in which, Sirt1 levels were knocked down. To validate further these visual results, we measured triglyceride content and free fatty acid (FFA) release in these cells. Triglyceride content was reduced and release of FFA was stimulated by resveratrol in the control cells, but not in the Sirt1 knockdown cells. These findings strongly suggest that upregulation of Sirt1 stimulates fat mobilization in fully differentiated 3T3-L1 adipocytes.

To determine whether Sirt1 stimulates fat mobilization in bona fide adipocytes, we cultured primary rat white adipocytes and activated fat mobilization with the known β-adrenergic inducer adrenalin. Addition of resveratrol greatly stimulated the release of FFA triggered by adrenalin, consistent with the findings in the 3T3-L1 cells. In a converse experiment using these rat adipocytes, we addressed whether inhibition of Sirt1 would blunt the mobilization of fat by adrenalin. Addition of the known Sirt1 inhibitor nicotinamide16 indeed reduced the release of FFA triggered by adrenalin. The above findings indicate that Sirt1 not only represses the differentiation program of adipocytes, but also activates the mobilization of fat in fully differentiated cells.

Sirt1 is an NAD-dependent deacetylase that can repress activity of p53 and forkhead proteins. Repression of adipogenesis and fat retention in 3T3-L1 cells by Sirt1 might be explained by inhibition of another transcription factor, PPAR-γ, as its activity is crucial for differentiation and maintenance of adipocytes. Thus, we determined by chromatin immunoprecipitation (ChIP) assays whether Sirt1 binds to PPAR-gamma sites in the promoters of the Ap2 and Pparg genes. In 3T3-L1 cells, Sirt1 and PPAR-gamma were both bound to similar promoter regions of Ap2 and Pparg. The interaction of Sirt1 with both promoter sequences was stronger in Sirt1-overexpressing cells and was lost in Sirt1 knockdown cells. Binding of Sirt1 to a control DNA region 2.5 kilobases (kb) upstream of the PPAR-gamma site near Pparg was not observed. Furthermore, luciferase reporter assays showed that Sirt1 repressed transactivation by PPAR-gamma. These findings indicate that Sirt1 and PPAR-gamma bind to the same DNA sequences and suggest that Sirt1 is a co-repressor of PPAR-γ.

To gain further evidence that Sirt1 is a PPAR-gamma co-repressor, we next determined whether the two proteins interact. Sirt1 was detected in PPAR-gamma immunoprecipitates from differentiated 3T3-L1 adipocytes. The PPAR-gamma cofactor NCoR20 was also co-immunoprecipitated by anti-Sirt1 antiserum but not by pre-immune serum. Glutathione S-transferase (GST) pull-down experiments revealed that two NCoR fragments interact with Sirt1: repression domain 1 (RD1) and the CBF/Su(H) interaction domain. Reciprocally, NCoR RD1 was found to interact with the amino-terminal region of Sirt1 (amino acids 1-214, GST-Sirt1 (Nt)), and the CBF1/Su(H) interaction domain of NCoR interacts with the homology domain of Sirt1 (amino acids 214-541, GST-Sirt1(SHD)). In analogous experiments, GST pull-down assays revealed an interaction between the Sirt1 homology domain and SMRT.

The interaction between Sirt1 and NcoR (and SMRT) suggests that Sirt1 represses PPAR-gamma activity by docking with the cofactors. Consistent with this, ChIP assays revealed that NCoR also binds to known PPAR-gamma sites of promoters of adipogenic genes. To test the possibility that Sirt1 functionally represses PPAR-gamma by means of NCoR, we infected Sirt1-overexpressing 3T3-L1 fibroblasts with an NCoR RNAi virus. Western blots showed that doubly infected cells overexpressed Sirt1 and underexpressed NCoR. As expected, overexpression of Sirt1 in the absence of the NCoR RNAi virus prevents fat accretion. In contrast, this reduction was largely prevented on simultaneous treatment with NCoR RNAi demonstrating that NCoR is required for repression of fat accumulation by Sirt1.

To test for a role of Sirt1 in fat mobilization in vivo, we first determined whether Sirt1 was expressed in WAT in mice. Sirt1 was found in all white adipose depots examined, with no notable distribution differences. Next, to probe for an in vivo function, we used mice with germline mutations in the Sirt1 gene. Unfortunately, the total absence of Sirt1 in mice (Sirt1−/−) results in a high degree of post-natal lethality and other severe phenotypes, precluding their use in this study. Therefore, we compared wild type to Sirt1±mice, which are phenotypically normal. No significant differences in epididymal WAT mass were observed between the wild type and heterozygous cohorts. As yeast Sir2 is important during calorie restriction, we assayed by ChIP the recruitment of Sirt1 to PPAR-γ-binding sites in the Ap2 and PPAR-gamma promoters in WAT of mice that were either fed or fasted. In mice fed ad libitum, Sirt1 was not bound to Ap2 or PPAR-gamma promoter sequences. However, Sirt1 was bound to these sequences after overnight food deprivation, showing that Sirt1 is recruited to PPAR-γ-binding sites of PPAR-gamma promoters upon fasting.

As a test of the effect of Sirt1 on fatty acid mobilization in adipocytes, we next addressed whether fatty acid release from WAT upon fasting was altered in Sirt1 /−mice. Heterozygous gene ablation was associated with a 40-45% lowering in circulating FFA levels in the blood after overnight food deprivation compared with wild type (P<0.05). To verify that these effects of Sirt1 genotype were due to differences in fat release from WAT and not re-uptake of FFA from the blood by oxidative tissues, we cultured the same number of white adipocytes from Sirt1+/+ and Sirt1/−mice, challenged them with adrenalin, and measured the release of FFA. Again, FFA release was reduced in the Sirt1±cells compared with the Sirt1+/+cells.

The mammalian Sir2 orthologue, Sirt1, is activated by food deprivation to trigger fat mobilization in WAT. The pharmacological activation of Sirt1 also elicits the lipolysis of triglycerides and the release of FFA. Because a reduction in fat storage in WAT is a primary way by which calorie restriction extends lifespan in mammals, our results provide a possible mechanism for understanding the regulation of mammalian lifespan by diet. Sirt1 represses WAT by inhibiting the transcription factor PPAR-γ. Starvation of animals causes Sirt1 to interact with PPAR-gamma promoter sites and thereby repress target genes that drive fat storage.

The pathway of regulation described here may impact on age-related diseases. The accumulation of WAT during ageing is associated with several adverse complications, such as insulin resistance, type 2 diabetes and atherosclerosis. Given the impact of Sirt1 on PPAR-gamma activity and because PPAR-gamma activity helps determine age-related insulin resistance, Sirt1 may have an important role in metabolic diseases and link the effects of food consumption to body fat mass and diseases of ageing. It is likely that calorie restriction exerts other effects on mammals to increase longevity, besides reducing WAT, as longevity in mice with reduced fat is not as great as animals on a long-term calorie restriction regimen. Tissues that metabolize fat and carbohydrate may also be important in delivering some of the benefit of calorie restriction.

Methods

Animal Experimentation

Wild-type FVB male age-matched (12-16 weeks old) mice were used for the present studies. Sirt1+/+ and Sirt1±genotypes have been described previously21. All mice were housed under controlled temperature (25±1° C.) and lighting conditions. Food provided was normal chow. All mice were cared for in accordance with the MIT animal care committee. Blood was collected from the retro-orbital sinus and kept on ice until centrifugation (1,500 g, 15 min at 4 ° C.), and the plasma was stored at −20 ° C. until analysis. All animals were killed by decapitation. WAT depots were collected, weighed and quickly frozen in liquid nitrogen and stored at −70° C. until further processing by immunoblotting. Non-esterified free fatty acids were determined by enzymatic assays (Wako Pure Chemical Industries).

Cell culture, retroviral infection and transfection. 3T3-L1, HEK293, 293T and Phoenix cells (ATCC, Rockville, Md.) were cultured in Dulbecco's modified Eagle's medium with 10% FCS, and antibiotics. Primary adipocytes from Sprague-Dawley rats were prepared as described previously25. Transfections for luciferase assays were done as described using pSPORT6-PPAR-γ2, pGL3-PPRE26 and pcDNA3-Sirt1. Data were corrected for transfection efficiency.

Retroviral infection was performed as described in Tontonoz et al. (1994) Cell 79:1147. Phoenix cells were transfected with either pBABE, pBABE-Sirt1, pSUPERretro (Oligoengine), pSUPERretro-Sirt1 RNAi (5′-GATGAAGTTGACCTCCTCA-3′, SEQ ID NO:24) or pSUPER-NCoR RNAi (5′-GCTGCATCCAAGGGCCATG-3′, SEQ ID NO:25) using Lipofectamine (Invitrogen). After 48 h of transfection, the medium containing retroviruses was collected, filtered, treated by polybrene (1 μg ml⁻¹) and transferred to 3T3-L1 target cells. Infected cells were selected with puromycin (2.5 μg ml⁻¹) for 7 days.

To stimulate adipogenesis and accumulation of lipids in cells, medium was supplemented to confluent cells (day 0) with 2 μM insulin or 10⁻⁷ M rosiglitazone (Alexis Biochemicals), as stated in the text, 1 μM dexamethasone, and 0.25 mM isobuthylmethylxanthine (IBMX) for 2 days. The cells were then incubated with insulin or 10-7 M rosiglitazone, changing the medium every second day. Fat accumulation was visualized by staining of lipids with Oil red O. Intracellular triglyceride levels were measured in cell lysates by an enzymatic method using a reagent kit from Boehringer Mannheim.

RNA and Protein Preparation and Analysis

Total RNA from cultured cells were extracted (Qiagen) and analyzed by semi-quantitative polymerase chain reaction with reverse transcription. GAPDH and 18S RNA levels were determined as a control for loading. Proteins from mouse tissues were extracted in a solution of pH 7.4 containing 20 mM HEPES, 250 mM sucrose, 4 mM EDTA, 1% Triton and protease inhibitor cocktail. 3T3-L1 cells were lysed in NET-N buffer (20 mM Tris-HCl, pH 8 containing 150 mM NaCl, 0.5% NP-40, 10% glycerol, 1 mM EDTA and a protease inhibitor cocktail).

Chromatin immunoprecipitation. For in vitro ChIP, infected 3T3-L1 cells were differentiated as mentioned above. For in vivo ChIP, epididymal WAT was dissected, minced and fixed overnight in PBS containing 1% formaldehyde and protease inhibitor cocktail. Tissues were then rinsed five times in PBS. Further ChIP assays were performed as described previously, using 1:200 antibody dilutions to immunoprecipitate DNA-protein complexes. DNA was then purified using Qiagen PCR purification kit and PCR reaction was performed using primers for Ap2 (5′-AAATTCAGAAGAAAGTAAACACATTATT-3′, SEQ ID NO:26; 5′-ATGCCCTGACCATGTGA-3′, SEQ ID NO:27) and PPAR-gamma proximal (amplifying a region at 0.3 kb upstream of ATG: 5′-GAGCAAGGTCTTCATCATTACG-3′, SEQ ID NO:28; 5′-CCCCTGGAGCTGGAGTTAC-3′, SEQ ID NO:29) and distal (amplifying a region at 2.8 kb upstream of ATG: 5′-CTCTCCCACCCTCGCCATAC-3′, SEQ ID NO:3-; 5′-TTGCCAGAGAAGCCAGTGACA-3′, SEQ ID NO:31) promoters.

Pull-down and co-immunoprecipitation assays. Differentiated 3T3-L1 or HEK293 cells were lysed in NET-N buffer by agitation at 4° C. for 30 min. After a brief sonication, lysates were cleared by centrifugation and immunoprecipitated. Antibodies used were anti-Sirt1 (Upstate), anti-NCoR (Upstate), anti-PPAR-gamma (SantaCruz), anti-C/EBP-alpha (SantaCruz) and anti-Sirt1 antiserum29 or preimmune serum. Immunoprecipitates were then analysed by immunoblotting. Equivalent amounts of GST or GST-Sirt1 fusion proteins were bound to glutathione-Sepharose (Pharmacia) and incubated with 35S-methionine-labelled NCoR or SMRT fragments prepared using TNT transcription-translation system (Promega). The reactions were washed five times with binding buffer (10 mM Na-HEPES containing 10% glycerol, 1 mM EDTA, 1 mM DTT, 150 mM NaCl and 0.05% NP-40) and bound proteins were eluted and resolved on denaturing SDS-polyacrylamide gel electrophoresis gels for analysis by autoradiography.

Statistical analysis. The main and interactive effects were analyzed by analysis of variance (ANOVA) factorial or repeated measures when appropriate. When justified by the ANOVA analysis, differences between individual group means were analyzed by Fisher's PLSD test. Differences were considered statistically significant at P<0.05.

### EXAMPLE

Further observations were made. A mouse genetically deficient for SIRT1 activity cannot regulate body temperature in the cold. The mouse appears unable to induce the UCP1 gene or related transcriptional programs. We used chromatin immunoprecipitations to characterize genes which may include regulatory sequences that are directly or indirectly bound by SIRT1. We discovered that, at least in brown fat cells, the UCP1 promoter is bound by SIRT1. SIRT1 responds to stress and activates UCP1 transcription. At least in part, the transcriptional activation is a result of the PGC1 co-factor, which is present in BAT cells, but absent in WAT cells. PGC1 modifies SIRT1 from a repressor to an activator. PGC1, SIRT1, and PPARγ are able to form a ternary complex which functions as a transcriptional activator. SIRT1 may have a similar function in other cells that express PGC1, e.g., muscle and liver cells.

Moreover, resveratrol increases UCP1 expression in BAT cells.

Increasing SIRT1 activity in WAT cells contributes to shedding of fat into the blood and the burning of fat in BAT. These activities can increase insulin sensitivity and can be used, e.g., to treated Type II diabetes, e.g., by decreasing insulin resistance.

A number of embodiments of the invention have been described. Nevertheless, it will be understood that various modifications may be made without departing from the spirit and scope of the invention. Accordingly, other embodiments are within the scope of the following claims.

1. A method of evaluating a subject, the method comprising:
evaluating a SIRT1 molecule from a subject; and
recording information from the SIRT1 evaluation in association with metabolic information about the subject.

2-6. (canceled)

7. The method of claim 1 wherein the evaluating comprises evaluating a cell of the adipose lineage.

8-14. (canceled)

15. A method of evaluating a subject who is being treated for a metabolic condition, the method comprising:
treating a subject with a regimen for altering a metabolic condition;
before, during, or after the regimen, monitoring a parameter associated with a SIRT1 molecule from the subject; and
comparing results of the evaluation to reference information to provide an assessment of the subject.

16-22. (canceled)

23. A method of evaluating a subject, the method comprising:
evaluating a SIRT1 molecule from a cell of the adipocyte lineage; and
comparing results of the evaluating to reference information.

24-36. (canceled)

37. A method comprising:
providing a pre-adipocyte or an adipocyte cell; and
evaluating expression or activity of a SIRT1 gene in the pre-adipocyte or adipocyte cell.

38-49. (canceled)

51. A method comprising:
identifying a subject has having obesity, being at risk for obesity using clinical criteria, or being overweight;
obtaining a sample of cells from the subject; and
evaluating expression of a SIRT1 gene in cells of the sample.

52-54. (canceled)

55. A method comprising:
identifying a subject as having obesity, being at risk for obesity using clinical criteria, or being overweight; and
administering an effective amount of an agent that increases SIRT1 activity to the subject.

56-67. (canceled)

68. A method comprising:
identifying a subject as being underweight, at risk for weight loss or cachexia using clinical criteria, or being cachexic; and
administering an effective amount of an agent that decreases SIRT1 activity to the subject.

69-78. (canceled)

79. A method of evaluating a compound, the method comprising:
providing a compound that interacts with SIRT1 or that modulates SIRT1 activity;
contacting the compound to a cell of the adipocyte lineage; and
evaluating the cell.

80. The method of claim 79 wherein evaluating comprises evaluating expression of a gene regulated by an adipocyte transcription factor.

81. The method of claim 80 wherein the adipocyte transcription factor is a PPAR transcription factor, PGC1, or a C/EBP transcription factor.

82. The method of claim 80 wherein the cell comprises a reporter gene regulated by the adipocyte transcription factor and the evaluating comprises evaluating the reporter gene.

83. The method of claim 79 wherein evaluating comprises evaluating the differentiation state of the cell.

84. The method of claim 79 wherein evaluating comprises evaluating secretion of a hormone by the cell.

85. The method of claim 79 wherein evaluating comprises evaluating fat mobilization by the cell.

86. The method of claim 79 wherein evaluating comprises evaluating fat burning by the cell.

87. The method of claim 79 wherein evaluating comprises evaluating association of SIRT1 and genomic nucleic acid in the cell.

88-89. (canceled)

90. A method of evaluating a compound, the method comprising:
contacting the compound to a preadipocyte cell; and
evaluating a parameter associated with a SIRT1 molecule of the cell

91. The method of claim 90 further comprising comparing the parameter to a reference parameter.

92. The method of claim 91 wherein the reference parameter is determined by a corresponding method for a preadipocyte that has not been contacted with the compound and a difference in the parameter and a reference parameter indicates that the compound alters SIRT1 activity in the preadipocyte.

93. The method of claim 90 further comprising evaluating the differentiation state of the predadipocyte cell.

94. The method of claim 90 further comprising evaluating lipid or fat of the preadipocyte cell.

95. The method of claim 94 wherein the evaluating comprises fractionating cell contents or an optical evaluation.

96. A method of evaluating a compound, the method comprising:
contacting the compound to an organism; and
evaluating a parameter associated with a SIRT1 molecule, from the organism, wherein a difference in the parameter between the parameter and a reference parameter indicates that the compound modulates SIRT1 activity in a cell of the organism.

97. A method of evaluating a compound comprising:
contacting the compound to a SIRT1 protein in vitro;
evaluating an interaction between the compound and the protein;
contacting the compound to a cell or organism; and
evaluating a differentiation state of the cell or a metabolic parameter of the organism.

98. The method of claim 97 wherein evaluating the interaction comprises evaluating catalytic activity of the protein in the presence of the compound.

99. A method of evaluating a library of compounds, the method comprising:
providing a library of compound;
for each compound of a plurality of compounds from the library,
contacting the compound to a SIRT1 protein in vitro;
evaluating an interaction between the compound and the SIRT1 protein;
if the compound interacts with the SIRT1 protein, contacting the compound to a cell or organism; and
evaluating a differentiation state or a metabolic parameter of the cell or organism.

100. The method of claim 99 wherein the cell includes a reporter gene and/or other combination of heterologous nucleic acids described herein.

101. A method of evaluating a library of compounds, the method comprising:
providing a library of compound;
for each compound of a plurality of compounds from the library, evaluating the compound using a method described herein.

102-119. (canceled)

120. A method comprising:
providing a mammalian adipocyte or pre-adipocyte cell; and
modulating SIRT1 activity in the cell.

121. The method of claim 120 wherein the modulating comprises increasing SIRT1 activity.

122. The method of claim 120 wherein the modulating comprises decreasing SIRT1 activity.

123. The method of claim 120 wherein the modulating comprises contacting the cell with a dsRNA.

124. The method of claim 120 wherein the modulating comprises introducing a nucleic acid that comprises a sequence that encodes a polypeptide comprising a SIRT1 core domain or a sequence complementary to a SIRT1 coding sequence.

125. The method of claim 120 wherein the adipocyte is a WAT or BAT cell.

126. A method comprising:
providing a mammalian cell;
modulating SIRT1 activity in the cell; and
evaluating a lipid or fat-associated parameter of the cell.

127. The method of claim 126 wherein the evaluating comprises an optical evaluation of the cell.

128. A method comprising:
providing a mammalian cell;
modulating SIRT1 activity in the cell; and
evaluating the differentiation state of the cell using an indicator of adipocyte differentiation.

129. The method of claim 128 wherein the indicator is leptin expression.

130. The method of claim 128 wherein the indicator is expression or activity of an adipocyte transcription factor.

131. The method of claim 128 wherein the cell contains a heterologous nucleic acid that can express a C/EBP protein and a reporter nucleic acid that comprises a regulatory sequence of gene that is specifically or selectively expressed in adipocytes.

132. The method of claim 128 wherein the cell contains a heterologous nucleic acid that can express a C/EBP protein and a reporter nucleic acid that comprises a regulatory sequence of a secrete protein produced by adipocytes.

133. The method of claim 132 wherein the secreted protein produced by adipocytes is leptin.

134. The method of claim 131 wherein the C/EBP protein is C/EBPa.

135. The method of claim 128 wherein the cell contains a heterologous nucleic acid that can express a PPAR protein and a reporter nucleic acid that comprises a regulatory sequence that is bound by an AP2 protein.

136. The method of claim 127 wherein the PPAR protein is PPAR-gamma.
